SEQUENCE LISTING (381 sequences)

SEQ ID NO: 1 - protein, 11 aa, Oryctolagus cuniculus
RASQTIYKNLH

SEQ ID NO: 2 - protein, 7 aa, Oryctolagus cuniculus
YGSDSIS

SEQ ID NO: 3 - protein, 9 aa, Oryctolagus cuniculus
LQGYSTPWT

SEQ ID NO: 4 - protein, 6 aa, Oryctolagus cuniculus
TDYSMH

SEQ ID NO: 5 - protein, 17 aa, Oryctolagus cuniculus
WINTATGEPTYADDFKG

SEQ ID NO: 6 - protein, 4 aa, Oryctolagus cuniculus
YYDY

SEQ ID NO: 7 - protein, 23 aa, Homo sapiens
EIVMTQSPSTLSASVGDRVIITC

SEQ ID NO: 8 - protein, 15 aa, Homos sapiens
WYQQKPGKAPKLLIY

SEQ ID NO: 9 - protein, 32 aa, Homos sapiens
GVPSRFSGSGSGTEFTLTISSLQPDDFATYYC

SEQ ID NO: 10 - protein, 11 aa, Homos sapiens
FGQGTKLTVLG

SEQ ID NO: 11 - protein, 20 aa, Unknown
  Recombinant
GGGGSGGGGSGGGGSGGGGS

SEQ ID NO: 12 - protein, 26 aa, Homos sapiens
EVQLVESGGGLVQPGGSLRLSCTASG

SEQ ID NO: 13 - protein, 14 aa, Homos sapiens
WVRQAPGKGLEWVG

SEQ ID NO: 14 - protein, 32 aa, Homos sapiens
RFTISRDTSKNTVYLQMNSLRAEDTAVYYCAR

SEQ ID NO: 15 - protein, 11 aa, Homos sapiens
WGQGTLVTVSS

SEQ ID NO: 16 - protein, 23 aa, Unknown
  Recombinant
EIVMTQSPSTLSASVGDRVIITC

SEQ ID NO: 17 - protein, 15 aa, Unknown
  Recombinant
WYQQKPGKSPKLLIK

SEQ ID NO: 18 - protein, 15 aa, Unknown
  Recombinant
WYQQKPGKAPKLLIK

SEQ ID NO: 19 - protein, 15 aa, Unknown
  Recombinant
WYQQKPGKSPKLLIY

SEQ ID NO: 20 - protein, 15 aa, Unknown
  Recombinant
WYQQKPGKSPKLLIK

SEQ ID NO: 21 - protein, 32 aa, Unknown
  Recombinant
GVPSRFSGSGSGTEYTLTISSLQPDDFATYYC

SEQ ID NO: 22 - protein, 11 aa, Unknown
  Recombinant
FGQGTKLTVLG

SEQ ID NO: 23 - protein, 29 aa, Unknown
  Recombinant
EVQLVESGGGLVQPGGSLRLSCTASGYTF

SEQ ID NO: 24 - protein, 14 aa, Unknown
  Recombinant
WVRQAPGKGLEWVG

SEQ ID NO: 25 - protein, 32 aa, Unknown
  Recombinant
RFTISLDTSKNTVYLQMNSLRAEDTAVYFCAS

SEQ ID NO: 26 - protein, 32 aa, Unknown
  Recombinant
RFTISLDTSKNTVYLQMNSLRAEDTAVYYCAS

SEQ ID NO: 27 - protein, 9 aa, Unknown
  Recombinant
WGQGTLVTV

SEQ ID NO: 28 - protein, 235 aa, Unknown
  Recombinant
MQVKLKESGPELKKPGETVKISCKASGYTFTDYSMHWVKKTPGKGLKWLGWINTATGEPTYADDFKGRFAISLETSARTVYLQINNLRNEDTATYFCFSYYDYWGQGTTVTVSSGGGGSGGGGSGGGGDIKLSQSPSILSVTPGETVSLSCRASQTIYKNLHWYQQKSHRSPRLLIKYGSDSISGIPSRFTGSGSGTDYTLNINSVKPEDQGIYYCLQGYSTPWTFGGGTKLEIK

SEQ ID NO: 29 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISRDTSKNTVYLQMNSLRAEDTAVYYCARYYDYWGQGTLVTVSS

SEQ ID NO: 30 - protein, 242 aa, Unknown
  Recombinant
MDIKLTQSPSILSASVGDRVIITCRASQTIYKNLHWYQQKPHRSPRLLIKYGSDSISGIPSRFSGSGSGTEYTLTISSLQPDDFAIYYCLQGYSTPWTFGGGTKLTVLGGGGGSGGGGSGGGGSGGGGSQVQLVESGGGLVQPGGSLRLSCTASGYTFTDYSMHWVRKTPGKGLKWVGWINTATGEPTYADDFKGRFTISLDTSKNTVYLQMNSLRAEDTATYFCFSYYDYWGQGTTVTVSS

SEQ ID NO: 31 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 32 - protein, 242 aa, Unknown
  Recombinant
MDIVLTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISLDTSKNTVYLQMNSLRAEDTAVYFCASYYDYWGQGTLVTVSS

SEQ ID NO: 33 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYFCASYYDYWGQGTLVTVSS

SEQ ID NO: 34 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYYCASYYDYWGQGTLVTVSS

SEQ ID NO: 35 - protein, 242 aa, Unknown
  Recombinant
MEIVMTQSPSTLSASVGDRVIITCRASQTIYKNLHWYQQKPGKAPKLLIKYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISRDTSKNTVYLQMNSLRAEDTAVYFCASYYDYWGQGTLVTVSS

SEQ ID NO: 36 - protein, 242 aa, Unknown
  Recombinant
MDIVLTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 37 - protein, 242 aa, Unknown; identical to SEQ ID NO: 30
  Recombinant

SEQ ID NO: 38 - protein, 242 aa, Unknown; identical to SEQ ID NO: 31
  Recombinant

SEQ ID NO: 39 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 40 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYFCASYYDYWGQGTLVTVSS

SEQ ID NO: 41 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYFCASYYDYWGQGTLVTVSS

SEQ ID NO: 42 - protein, 242 aa, Unknown
  Recombinant
MDIVLTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYFCASYYDYWGQGTLVTVSS

SEQ ID NO: 43 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 44 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYYCASYYDYWGQGTLVTVSS

SEQ ID NO: 45 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 46 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 47 - protein, 242 aa, Unknown
  Recombinant
MDIVLTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 48 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 49 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 50 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYFCASYYDYWGQGTLVTVSS

SEQ ID NO: 51 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISLDTSKNTVYLQMNSLRAEDTAVYYCASYYDYWGQGTLVTVSS

SEQ ID NO: 52 - protein, 242 aa, Unknown
  Recombinant
MEIVMTQSPSTLSASVGDRVIITCRASQTIYKNLHWYQQKPGKSPKLLIYYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 53 - protein, 242 aa, Unknown
  Recombinant
MEIVMTQSPSTLSASVGDRVIITCRASQTIYKNLHWYQQKPGKAPKLLIKYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 54 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 55 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISLDTSKNTVYLQMNSLRAEDTAVYFCASYYDYWGQGTLVTVSS

SEQ ID NO: 56 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYYCASYYDYWGQGTLVTVSS

SEQ ID NO: 57 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLGGGGGSGGGGSGGGGSGGGGSQVQLVESGGGLVQPGGSLRLSCTASGYTFTDYSMHWVRKAPGKGLEWVGWINTATGEPTYADDFKGRFTISLDTSKNTVYLQMNSLRAEDTAVYFCASYYDYWGQGTLVTVSS

SEQ ID NO: 58 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKVEIKGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 59 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLEIKGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 60 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGPGTKVDIKGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 61 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGGGTKVEIKGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 62 - protein, 242 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTRLEIKGGGGGSGGGGSGGGGSGGGGSQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 63 - protein, 242 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLEIKGGGGGSGGGGSGGGGSGGGGSEVQLVESGGGLVQPGGSLRLSCTASGYTFTDYSMHWVRQAPGKGLEWVGWINTATGEPTYADDFKGRFTISLDTSKNTVYLQMNSLRAEDTAVYYCASYYDYWGQGTLVTVSS

SEQ ID NO: 64 - protein, 242 aa, Unknown
  Recombinant
MQVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSSGGGGSGGGGSGGGGSGGGGSEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLG

SEQ ID NO: 65 - protein, 242 aa, Unknown
  Recombinant
MEVQLVESGGGLVQPGGSLRLSCTASGYTFTDYSMHWVRQAPGKGLEWVGWINTATGEPTYADDFKGRFTISLDTSKNTVYLQMNSLRAEDTAVYYCASYYDYWGQGTLVTVSSGGGGSGGGGSGGGGSGGGGSEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLG

SEQ ID NO: 66 - protein, 107 aa, Unknown
  Recombinant
DIKLSQSPSILSVTPGETVSLSCRASQTIYKNLHWYQQKSHRSPRLLIKYGSDSISGIPSRFTGSGSGTDYTLNINSVKPEDQGIYYCLQGYSTPWTFGGGTKLEIK

SEQ ID NO: 67 - protein, 107 aa, Unknown
  Recombinant
DILLTQSPAILSVTPGETVSLSCRASQTIYKNLHWYQQKSHRSPRLLIKYGSDSISGIPSRFTGSGSGTDYTLNINSVKPEDEGIYYCLQGYSTPWTFGGGTKLEIK

SEQ ID NO: 68 - protein, 109 aa, Unknown
  Recombinant
MEIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLG

SEQ ID NO: 69 - protein, 109 aa, Unknown; identical to SEQ ID NO: 68
  Recombinant

SEQ ID NO: 70 - protein, 109 aa, Unknown
  Recombinant
MEIVMTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLG

SEQ ID NO: 71 - protein, 109 aa, Unknown
  Recombinant
MEIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLG

SEQ ID NO: 72 - protein, 108 aa, Unknown
  Recombinant
EIVMTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLG

SEQ ID NO: 73 - protein, 108 aa, Unknown
  Recombinant
DIKLTQSPSILSASVGDRVIITCRASQTIYKNLHWYQQKPHRSPRLLIKYGSDSISGIPSRFSGSGSGTEYTLTISSLQPDDFAIYYCLQGYSTPWTFGGGTKLTVLG

SEQ ID NO: 74 - protein, 108 aa, Unknown; identical to SEQ ID NO: 72
  Recombinant

SEQ ID NO: 75 - protein, 108 aa, Unknown
  Recombinant
DIVLTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLG

SEQ ID NO: 76 - protein, 108 aa, Unknown
  Recombinant
EIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLTVLG

SEQ ID NO: 77 - protein, 108 aa, Unknown
  Recombinant
EIVMTQSPSTLSASVGDRVIITCRASQTIYKNLHWYQQKPGKSPKLLIKYGSDSISGVPSRFSGSGSGTEFTLTISSLQPDDFATYYCLQGYSTPWTFGQGTKLTVLG

SEQ ID NO: 78 - protein, 108 aa, Unknown
  Recombinant
EIVMTQSPSTLSASVGDRVIITCRASQTIYKNLHWYQQKPGKAPKLLIKYGSDSISGVPSRFSGSGSGTEFTLTISSLQPDDFATYYCLQGYSTPWTFGQGTKLTVLG

SEQ ID NO: 79 - protein, 108 aa, Unknown
  Recombinant
DIVLTQSPSTLSASVGDRVIITCRASQTIYKNLHWYQQKPGKAPKLLIYYGSDSISGVPSRFSGSGSGTEFTLTISSLQPDDFATYYCLQGYSTPWTFGQGTKLTVLG

SEQ ID NO: 80 - protein, 108 aa, Unknown; identical to SEQ ID NO: 73
  Recombinant

SEQ ID NO: 81 - protein, 108 aa, Unknown; identical to SEQ ID NO: 72
  Recombinant

SEQ ID NO: 82 - protein, 108 aa, Unknown
  Recombinant
EIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLTVLG

SEQ ID NO: 83 - protein, 108 aa, Unknown; identical to SEQ ID NO: 72
  Recombinant

SEQ ID NO: 84 - protein, 108 aa, Unknown; identical to SEQ ID NO: 77
  Recombinant

SEQ ID NO: 85 - protein, 108 aa, Unknown; identical to SEQ ID NO: 79
  Recombinant

SEQ ID NO: 86 - protein, 108 aa, Unknown; identical to SEQ ID NO: 76
  Recombinant

SEQ ID NO: 87 - protein, 108 aa, Unknown; identical to SEQ ID NO: 76
  Recombinant

SEQ ID NO: 88 - protein, 108 aa, Unknown; identical to SEQ ID NO: 76
  Recombinant

SEQ ID NO: 89 - protein, 108 aa, Unknown; identical to SEQ ID NO: 72
  Recombinant

SEQ ID NO: 90 - protein, 108 aa, Unknown; identical to SEQ ID NO: 79
  Recombinant

SEQ ID NO: 91 - protein, 108 aa, Unknown; identical to SEQ ID NO: 82
  Recombinant

SEQ ID NO: 92 - protein, 108 aa, Unknown; identical to SEQ ID NO: 82
  Recombinant

SEQ ID NO: 93 - protein, 108 aa, Unknown; identical to SEQ ID NO: 82
  Recombinant

SEQ ID NO: 94 - protein, 108 aa, Unknown; identical to SEQ ID NO: 82
  Recombinant

SEQ ID NO: 95 - protein, 108 aa, Unknown
  Recombinant
EIVMTQSPSTLSASVGDRVIITCRASQTIYKNLHWYQQKPGKSPKLLIYYGSDSISGVPSRFSGSGSGTEYTLTISSLQPDDFATYYCLQGYSTPWTFGQGTKLTVLG

SEQ ID NO: 96 - protein, 108 aa, Unknown
  Recombinant
EIVMTQSPSTLSASVGDRVIITCRASQTIYKNLHWYQQKPGKAPKLLIKYGSDSISGVPSRFSGSGSGTEYTLTISSLQPDDFATYYCLQGYSTPWTFGQGTKLTVLG

SEQ ID NO: 97 - protein, 108 aa, Unknown; identical to SEQ ID NO: 82
  Recombinant

SEQ ID NO: 98 - protein, 108 aa, Unknown; identical to SEQ ID NO: 82
  Recombinant

SEQ ID NO: 99 - protein, 108 aa, Unknown; identical to SEQ ID NO: 82
  Recombinant

SEQ ID NO: 100 - protein, 108 aa, Unknown; identical to SEQ ID NO: 76
  Recombinant

SEQ ID NO: 101 - protein, 108 aa, Unknown
  Recombinant
EIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKVEIKG

SEQ ID NO: 102 - protein, 108 aa, Unknown
  Recombinant
EIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTKLEIKG

SEQ ID NO: 103 - protein, 108 aa, Unknown
  Recombinant
EIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGPGTKVDIKG

SEQ ID NO: 104 - protein, 108 aa, Unknown
  Recombinant
EIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGGGTKVEIKG

SEQ ID NO: 105 - protein, 108 aa, Unknown
  Recombinant
EIVMTQSPSTLSASVGDRVIITCRASQTIYKNLHWYQQKPGKSPKLLIKYGSDSISGVPSRFSGSGSGTEYTLTISSLQPDDFATYYCLQGYSTPWTFGQGTRLEIKG

SEQ ID NO: 106 - protein, 108 aa, Unknown
  Recombinant
EIVMTQSPSTLSASVGDRVIITCRASQTIYKNLHWYQQKPGKAPKLLIYYGSDSISGVPSRFSGSGSGTEYTLTISSLQPDDFATYYCLQGYSTPWTFGQGTKLEIKG

SEQ ID NO: 107 - protein, 108 aa, Unknown; identical to SEQ ID NO: 76
  Recombinant

SEQ ID NO: 108 - protein, 108 aa, Unknown; identical to SEQ ID NO: 82
  Recombinant

SEQ ID NO: 109 - protein, 113 aa, Unknown
  Recombinant
QVKLKESGPELKKPGETVKISCKASGYTFTDYSMHWVKKTPGKGLKWLGWINTATGEPTYADDFKGRFAISLETSARTVYLQINNLRNEDTATYFCFSYYDYWGQGTTVTVSS

SEQ ID NO: 110 - protein, 113 aa, Unknown
  Recombinant
EVQLVESGGGLVQPGGSLRLSCTASGYTFTDYSMHWVRQAPGKGLEWVGWINTATGEPTYADDFKGRFTISLDTSKNTVYLQMNSLRAEDTAVYFCASYYDYWGQGTLVTVSS

SEQ ID NO: 111 - protein, 113 aa, Unknown
  Recombinant
QVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 112 - protein, 113 aa, Unknown; identical to SEQ ID NO: 111
  Recombinant

SEQ ID NO: 113 - protein, 113 aa, Unknown
  Recombinant
EVQLVESGGGLVQPGGSLRLSCTASGYTFTDYSMHWVRQAPGKGLEWVGWINTATGEPTYADDFKGRFTISLDTSKNTVYLQMNSLRAEDTAVYYCASYYDYWGQGTLVTVSS

SEQ ID NO: 114 - protein, 113 aa, Unknown
  Recombinant
QIQLVQSGPELKKPGETVKISCKASGYTFTDYSMHWVKKTPGKGLKWLGWINTATGEPTYADDFKGRFAISLETSARTVYLQINNLRNEDTATYFCFSYYDYWGQGTTLTVSS

SEQ ID NO: 115 - protein, 113 aa, Unknown
  Recombinant
EVQLVESGGGLVQPGGSLRLSCTASGYTFTDYSMHWVRQAPGKGLEWVGWINTATGEPTYADDFKGRFTISRDTSKNTVYLQMNSLRAEDTAVYYCARYYDYWGQGTLVTVSS

SEQ ID NO: 116 - protein, 113 aa, Unknown
  Recombinant
QVQLVESGGGLVQPGGSLRLSCTASGYTFTDYSMHWVRKTPGKGLKWVGWINTATGEPTYADDFKGRFTISLDTSKNTVYLQMNSLRAEDTATYFCFSYYDYWGQGTTVTVSS

SEQ ID NO: 117 - protein, 113 aa, Unknown
  Recombinant
EVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 118 - protein, 113 aa, Unknown
  Recombinant
QVQLVESGGGLVQPGGSLRLSCTASGYTFTDYSMHWVRKAPGKGLEWVGWINTATGEPTYADDFKGRFTISLDTSKNTVYLQMNSLRAEDTAVYFCASYYDYWGQGTLVTVSS

SEQ ID NO: 119 - protein, 113 aa, Unknown; identical to SEQ ID NO: 110
  Recombinant

SEQ ID NO: 120 - protein, 113 aa, Unknown; identical to SEQ ID NO: 113
  Recombinant

SEQ ID NO: 121 - protein, 113 aa, Unknown
  Recombinant
EVQLVESGGGLVQPGGSLRLSCTASGYTFTDYSMHWVRQAPGKGLEWVGWINTATGEPTYADDFKGRFTISRDTSKNTVYLQMNSLRAEDTAVYFCASYYDYWGQGTLVTVSS

SEQ ID NO: 122 - protein, 113 aa, Unknown; identical to SEQ ID NO: 111
  Recombinant

SEQ ID NO: 123 - protein, 113 aa, Unknown; identical to SEQ ID NO: 116
  Recombinant

SEQ ID NO: 124 - protein, 113 aa, Unknown; identical to SEQ ID NO: 117
  Recombinant

SEQ ID NO: 125 - protein, 113 aa, Unknown; identical to SEQ ID NO: 117
  Recombinant

SEQ ID NO: 126 - protein, 113 aa, Unknown; identical to SEQ ID NO: 110
  Recombinant

SEQ ID NO: 127 - protein, 113 aa, Unknown; identical to SEQ ID NO: 110
  Recombinant

SEQ ID NO: 128 - protein, 113 aa, Unknown; identical to SEQ ID NO: 110
  Recombinant

SEQ ID NO: 129 - protein, 113 aa, Unknown; identical to SEQ ID NO: 117
  Recombinant

SEQ ID NO: 130 - protein, 113 aa, Unknown; identical to SEQ ID NO: 113
  Recombinant

SEQ ID NO: 131 - protein, 113 aa, Unknown; identical to SEQ ID NO: 111
  Recombinant

SEQ ID NO: 132 - protein, 113 aa, Unknown; identical to SEQ ID NO: 111
  Recombinant

SEQ ID NO: 133 - protein, 113 aa, Unknown; identical to SEQ ID NO: 117
  Recombinant

SEQ ID NO: 134 - protein, 113 aa, Unknown
  Recombinant
EVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVSS

SEQ ID NO: 135 - protein, 113 aa, Unknown
  Recombinant
QVQLVESGGGLVQPGGSLRLSCTASGYTFTDYSMHWVRQAPGKGLEWVGWINTATGEPTYADDFKGRFTISRDTSKNTVYLQMNSLRAEDTAVYYCASYYDYWGQGTLVTVSS

SEQ ID NO: 136 - protein, 113 aa, Unknown
  Recombinant
QVQLVESGGGLVQPGGSLRLSCTASGYTFTDYSMHWVRKAPGKGLEWVGWINTATGEPTYADDFKGRFTISRDTSKNTVYLQMNSLRAEDTAVYFCASYYDYWGQGTLVTVSS

SEQ ID NO: 137 - protein, 113 aa, Unknown
  Recombinant
QVQLVESGGGLVQPGGSLRLSCTASGYTFTDYSMHWVRKAPGKGLEWVGWINTATGEPTYADDFKGRFTISLDTSKNTVYLQMNSLRAEDTAVYYCASYYDYWGQGTLVTVSS

SEQ ID NO: 138 - protein, 113 aa, Unknown; identical to SEQ ID NO: 111
  Recombinant

SEQ ID NO: 139 - protein, 113 aa, Unknown; identical to SEQ ID NO: 111
  Recombinant

SEQ ID NO: 140 - protein, 113 aa, Unknown; identical to SEQ ID NO: 111
  Recombinant

SEQ ID NO: 141 - protein, 113 aa, Unknown; identical to SEQ ID NO: 118
  Recombinant

SEQ ID NO: 142 - protein, 113 aa, Unknown; identical to SEQ ID NO: 113
  Recombinant

SEQ ID NO: 143 - protein, 113 aa, Unknown; identical to SEQ ID NO: 118
  Recombinant

SEQ ID NO: 144 - protein, 112 aa, Unknown
  Recombinant
QVQLVESGGGLVQPGGSLRLSCTASGYTFTDYSMHWVRKAPGKGLEWVGWINTATGEPTYADDFKGRFTISRDTSKNTVYLQMNSLRAEDTAVYYCASYYDYWGQGTLVTVS

SEQ ID NO: 145 - protein, 113 aa, Unknown; identical to SEQ ID NO: 111
  Recombinant

SEQ ID NO: 146 - protein, 113 aa, Unknown; identical to SEQ ID NO: 111
  Recombinant

SEQ ID NO: 147 - protein, 113 aa, Unknown; identical to SEQ ID NO: 111
  Recombinant

SEQ ID NO: 148 - protein, 113 aa, Unknown; identical to SEQ ID NO: 111
  Recombinant

SEQ ID NO: 149 - protein, 113 aa, Unknown; identical to SEQ ID NO: 113
  Recombinant

SEQ ID NO: 150 - protein, 113 aa, Unknown; identical to SEQ ID NO: 111
  Recombinant

SEQ ID NO: 151 - protein, 113 aa, Unknown; identical to SEQ ID NO: 113
  Recombinant

SEQ ID NO: 152 - protein, 23 aa, Unknown
  Recombinant
DIKLSQSPSILSVTPGETVSLSC

SEQ ID NO: 153 - protein, 23 aa, Unknown
  Recombinant
DIKLSQSPSILSVTPGETVSLSC

SEQ ID NO: 154 - protein, 23 aa, Unknown
  Recombinant
DIKLTQSPSILSASVGDRVIITC

SEQ ID NO: 155 - protein, 23 aa, Unknown
  Recombinant
DIVLTQSPSTLSASVGDRVIITC

SEQ ID NO: 156 - protein, 23 aa, Unknown
  Recombinant
EIVMTQSPSTLSASVGDRVIITC

SEQ ID NO: 157 - protein, 23 aa, Unknown
  Recombinant
DILLTQSPAILSVTPGETVSLSC

SEQ ID NO: 158 - protein, 23 aa, Unknown
  Recombinant
DILLSQSPSILSVTPGETVSLSC

SEQ ID NO: 159 - protein, 23 aa, Unknown
  Recombinant
DIKLTQSPSILSVTPGETVSLSC

SEQ ID NO: 160 - protein, 23 aa, Unknown
  Recombinant
DIKLSQSPAILSVTPGETVSLSC

SEQ ID NO: 161 - protein, 23 aa, Unknown
  Recombinant
DILLTQSPSILSVTPGETVSLSC

SEQ ID NO: 162 - protein, 23 aa, Unknown
  Recombinant
DIKLTQSPAILSVTPGETVSLSC

SEQ ID NO: 163 - protein, 23 aa, Unknown
  Recombinant
DILLSQSPAILSVTPGETVSLSC

SEQ ID NO: 164 - protein, 23 aa, Unknown
  Recombinant
DIALSQSPSILSVTPGETVSLSC

SEQ ID NO: 165 - protein, 23 aa, Unknown
  Recombinant
DICLSQSPSILSVTPGETVSLSC

SEQ ID NO: 166 - protein, 23 aa, Unknown
  Recombinant
DIDLSQSPSILSVTPGETVSLSC

SEQ ID NO: 167 - protein, 23 aa, Unknown
  Recombinant
DIELSQSPSILSVTPGETVSLSC

SEQ ID NO: 168 - protein, 23 aa, Unknown
  Recombinant
DIFLSQSPSILSVTPGETVSLSC

SEQ ID NO: 169 - protein, 23 aa, Unknown
  Recombinant
DIGLSQSPSILSVTPGETVSLSC

SEQ ID NO: 170 - protein, 23 aa, Unknown
  Recombinant
DIHLSQSPSILSVTPGETVSLSC

SEQ ID NO: 171 - protein, 23 aa, Unknown
  Recombinant
DIILSQSPSILSVTPGETVSLSC

SEQ ID NO: 172 - protein, 23 aa, Unknown
  Recombinant
DIKLSQSPSILSVTPGETVSLSC

SEQ ID NO: 173 - protein, 23 aa, Unknown
  Recombinant
DIMLSQSPSILSVTPGETVSLSC

SEQ ID NO: 174 - protein, 23 aa, Unknown
  Recombinant
DINLSQSPSILSVTPGETVSLSC

SEQ ID NO: 175 - protein, 23 aa, Unknown
  Recombinant
DIPLSQSPSILSVTPGETVSLSC

SEQ ID NO: 176 - protein, 23 aa, Unknown
  Recombinant
DIQLSQSPSILSVTPGETVSLSC

SEQ ID NO: 177 - protein, 23 aa, Unknown
  Recombinant
DIRLSQSPSILSVTPGETVSLSC

SEQ ID NO: 178 - protein, 23 aa, Unknown
  Recombinant
DISLSQSPSILSVTPGETVSLSC

SEQ ID NO: 179 - protein, 23 aa, Unknown
  Recombinant
DITLSQSPSILSVTPGETVSLSC

SEQ ID NO: 180 - protein, 23 aa, Unknown
  Recombinant
DIVLSQSPSILSVTPGETVSLSC

SEQ ID NO: 181 - protein, 23 aa, Unknown
  Recombinant
DIWLSQSPSILSVTPGETVSLSC

SEQ ID NO: 182 - protein, 23 aa, Unknown
  Recombinant
DIYLSQSPSILSVTPGETVSLSC

SEQ ID NO: 183 - protein, 23 aa, Unknown
  Recombinant
DIKLAQSPSILSVTPGETVSLSC

SEQ ID NO: 184 - protein, 23 aa, Unknown
  Recombinant
DIKLCQSPSILSVTPGETVSLSC

SEQ ID NO: 185 - protein, 23 aa, Unknown
  Recombinant
DIKLDQSPSILSVTPGETVSLSC

SEQ ID NO: 186 - protein, 23 aa, Unknown
  Recombinant
DIKLEQSPSILSVTPGETVSLSC

SEQ ID NO: 187 - protein, 23 aa, Unknown
  Recombinant
DIKLFQSPSILSVTPGETVSLSC

SEQ ID NO: 188 - protein, 23 aa, Unknown
  Recombinant
DIKLGQSPSILSVTPGETVSLSC

SEQ ID NO: 189 - protein, 23 aa, Unknown
  Recombinant
DIKLHQSPSILSVTPGETVSLSC

SEQ ID NO: 190 - protein, 23 aa, Unknown
  Recombinant
DIKLIQSPSILSVTPGETVSLSC

SEQ ID NO: 191 - protein, 23 aa, Unknown
  Recombinant
DIKLKQSPSILSVTPGETVSLSC

SEQ ID NO: 192 - protein, 23 aa, Unknown
  Recombinant
DIKLLQSPSILSVTPGETVSLSC

SEQ ID NO: 193 - protein, 23 aa, Unknown
  Recombinant
DIKLMQSPSILSVTPGETVSLSC

SEQ ID NO: 194 - protein, 23 aa, Unknown
  Recombinant
DIKLNQSPSILSVTPGETVSLSC

SEQ ID NO: 195 - protein, 23 aa, Unknown
  Recombinant
DIKLPQSPSILSVTPGETVSLSC

SEQ ID NO: 196 - protein, 23 aa, Unknown
  Recombinant
DIKLQQSPSILSVTPGETVSLSC

SEQ ID NO: 197 - protein, 23 aa, Unknown
  Recombinant
DIKLRQSPSILSVTPGETVSLSC

SEQ ID NO: 198 - protein, 23 aa, Unknown
  Recombinant
DIKLSQSPSILSVTPGETVSLSC

SEQ ID NO: 199 - protein, 23 aa, Unknown
  Recombinant
DIKLVQSPSILSVTPGETVSLSC

SEQ ID NO: 200 - protein, 23 aa, Unknown
  Recombinant
DIKLWQSPSILSVTPGETVSLSC

SEQ ID NO: 201 - protein, 23 aa, Unknown
  Recombinant
DIKLWQSPSILSVTPGETVSLSC

SEQ ID NO: 202 - protein, 23 aa, Unknown
  Recombinant
DIKLSQSPCILSVTPGETVSLSC

SEQ ID NO: 203 - protein, 23 aa, Unknown
  Recombinant
DIKLSQSPDILSVTPGETVSLSC

SEQ ID NO: 204 - protein, 23 aa, Unknown
  Recombinant
DIKLSQSPEILSVTPGETVSLSC

SEQ ID NO: 205 - protein, 23 aa, Unknown
  Recombinant
DIKLSQSPFILSVTPGETVSLSC

SEQ ID NO: 206 - protein, 23 aa, Unknown
  Recombinant
DIKLSQSPGILSVTPGETVSLSC

SEQ ID NO: 207 - protein, 23 aa, Unknown
  Recombinant
DIKLSQSPHILSVTPGETVSLSC

SEQ ID NO: 208 - protein, 23 aa, Unknown
  Recombinant
DIKLSQSPIILSVTPGETVSLSC

SEQ ID NO: 209 - protein, 23 aa, Unknown
  Recombinant
DIKLSQSPKILSVTPGETVSLSC

SEQ ID NO: 210 - protein, 23 aa, Unknown
  Recombinant
DIKLSQSPLILSVTPGETVSLSC

SEQ ID NO: 211 - protein, 23 aa, Unknown
  Recombinant
DIKLSQSPMILSVTPGETVSLSC

SEQ ID NO: 212 - protein, 23 aa, Unknown
  Recombinant
DIKLSQSPNILSVTPGETVSLSC

SEQ ID NO: 213 - protein, 23 aa, Unknown
  Recombinant
DIKLSQSPPILSVTPGETVSLSC

SEQ ID NO: 214 - protein, 23 aa, Unknown
  Recombinant
DIKLSQSPQILSVTPGETVSLSC

SEQ ID NO: 215 - protein, 23 aa, Unknown
  Recombinant
DIKLSQSPRILSVTPGETVSLSC

SEQ ID NO: 216 - protein, 23 aa, Unknown
  Recombinant
DIKLSQSPSILSVTPGETVSLSC

SEQ ID NO: 217 - protein, 23 aa, Unknown
  Recombinant
DIKLSQSPTILSVTPGETVSLSC

SEQ ID NO: 218 - protein, 23 aa, Unknown
  Recombinant
DIKLSQSPVILSVTPGETVSLSC

SEQ ID NO: 219 - protein, 23 aa, Unknown
  Recombinant
DIKLSQSPWILSVTPGETVSLSC

SEQ ID NO: 220 - protein, 23 aa, Unknown
  Recombinant
DIKLSQSPYILSVTPGETVSLSC

SEQ ID NO: 221 - protein, 15 aa, Unknown
  Recombinant
WYQQKSHRSPRLLIK

SEQ ID NO: 222 - protein, 7 aa, Unknown
  Recombinant
WYQQKPG

SEQ ID NO: 223 - protein, 7 aa, Unknown
  Recombinant
WYQQKPH

SEQ ID NO: 224 - protein, 7 aa, Unknown
  Recombinant
WYQQKSH

SEQ ID NO: 225 - protein, 32 aa, Unknown
  Recombinant
GIPSRFTGSGSGTDYTLNINSVKPEDQGIYYC

SEQ ID NO: 226 - protein, 32 aa, Unknown
  Recombinant
GIPSRFTGSGSGTDYTLNINSVKPEDEGIYYC

SEQ ID NO: 227 - protein, 32 aa, Unknown
  Recombinant
GIPSRFSGSGSGTEYTLTISSLQPDDFAIYYC

SEQ ID NO: 228 - protein, 32 aa, Unknown; identical to SEQ ID NO: 225
  Recombinant

SEQ ID NO: 229 - protein, 32 aa, Unknown; identical to SEQ ID NO: 9
  Recombinant

SEQ ID NO: 230 - protein, 32 aa, Unknown; identical to SEQ ID NO: 21
  Recombinant

SEQ ID NO: 231 - protein, 32 aa, Unknown
  Recombinant
GIPSRFTGSGSGTDYTLNINSVKPEDAGIYYC

SEQ ID NO: 232 - protein, 32 aa, Unknown
  Recombinant
GIPSRFTGSGSGTDYTLNINSVKPEDCGIYYC

SEQ ID NO: 233 - protein, 32 aa, Unknown
  Recombinant
GIPSRFTGSGSGTDYTLNINSVKPEDDGIYYC

SEQ ID NO: 234 - protein, 32 aa, Unknown
  Recombinant
GIPSRFTGSGSGTDYTLNINSVKPEDFGIYYC

SEQ ID NO: 235 - protein, 32 aa, Unknown
  Recombinant
GIPSRFTGSGSGTDYTLNINSVKPEDGGIYYC

SEQ ID NO: 236 - protein, 32 aa, Unknown
  Recombinant
GIPSRFTGSGSGTDYTLNINSVKPEDHGIYYC

SEQ ID NO: 237 - protein, 32 aa, Unknown
  Recombinant
GIPSRFTGSGSGTDYTLNINSVKPEDIGIYYC

SEQ ID NO: 238 - protein, 32 aa, Unknown
  Recombinant
GIPSRFTGSGSGTDYTLNINSVKPEDKGIYYC

SEQ ID NO: 239 - protein, 32 aa, Unknown
  Recombinant
GIPSRFTGSGSGTDYTLNINSVKPEDLGIYYC

SEQ ID NO: 240 - protein, 32 aa, Unknown
  Recombinant
GIPSRFTGSGSGTDYTLNINSVKPEDMGIYYC

SEQ ID NO: 241 - protein, 32 aa, Unknown
  Recombinant
GIPSRFTGSGSGTDYTLNINSVKPEDNGIYYC

SEQ ID NO: 242 - protein, 32 aa, Unknown
  Recombinant
GIPSRFTGSGSGTDYTLNINSVKPEDPGIYYC

SEQ ID NO: 243 - protein, 32 aa, Unknown
  Recombinant
GIPSRFTGSGSGTDYTLNINSVKPEDRGIYYC

SEQ ID NO: 244 - protein, 32 aa, Unknown
  Recombinant
GIPSRFTGSGSGTDYTLNINSVKPEDSGIYYC

SEQ ID NO: 245 - protein, 32 aa, Unknown
  Recombinant
GIPSRFTGSGSGTDYTLNINSVKPEDTGIYYC

SEQ ID NO: 246 - protein, 32 aa, Unknown
  Recombinant
GIPSRFTGSGSGTDYTLNINSVKPEDVGIYYC

SEQ ID NO: 247 - protein, 32 aa, Unknown
  Recombinant
GIPSRFTGSGSGTDYTLNINSVKPEDWGIYYC

SEQ ID NO: 248 - protein, 32 aa, Unknown
  Recombinant
GIPSRFTGSGSGTDYTLNINSVKPEDYGIYYC

SEQ ID NO: 249 - protein, 10 aa, Unknown
  Recombinant
FGGGTKLEIK

SEQ ID NO: 250 - protein, 11 aa, Unknown
  Recombinant
FGGGTKLTVLG

SEQ ID NO: 251 - protein, 11 aa, Unknown
  Recombinant
FGGGTKVEIKG

SEQ ID NO: 252 - protein, 11 aa, Unknown
  Recombinant
FGPGTKVDIKG

SEQ ID NO: 253 - protein, 11 aa, Unknown
  Recombinant
FGQGTKLEIKG

SEQ ID NO: 254 - protein, 11 aa, Unknown
  Recombinant
FGQGTKLTVLG

SEQ ID NO: 255 - protein, 29 aa, Unknown
  Recombinant
QAKLKESGPELKKPGETVKISCKASGYTF

SEQ ID NO: 256 - protein, 29 aa, Unknown
  Recombinant
QCKLKESGPELKKPGETVKISCKASGYTF

SEQ ID NO: 257 - protein, 29 aa, Unknown
  Recombinant
QDKLKESGPELKKPGETVKISCKASGYTF

SEQ ID NO: 258 - protein, 29 aa, Unknown
  Recombinant
QEKLKESGPELKKPGETVKISCKASGYTF

SEQ ID NO: 259 - protein, 29 aa, Unknown
  Recombinant
QFKLKESGPELKKPGETVKISCKASGYTF

SEQ ID NO: 260 - protein, 29 aa, Unknown
  Recombinant
QGKLKESGPELKKPGETVKISCKASGYTF

SEQ ID NO: 261 - protein, 29 aa, Unknown
  Recombinant
QHKLKESGPELKKPGETVKISCKASGYTF

SEQ ID NO: 262 - protein, 29 aa, Unknown
  Recombinant
QKKLKESGPELKKPGETVKISCKASGYTF

SEQ ID NO: 263 - protein, 29 aa, Unknown
  Recombinant
QLKLKESGPELKKPGETVKISCKASGYTF

SEQ ID NO: 264 - protein, 29 aa, Unknown
  Recombinant
QMKLKESGPELKKPGETVKISCKASGYTF

SEQ ID NO: 265 - protein, 29 aa, Unknown
  Recombinant
QNKLKESGPELKKPGETVKISCKASGYTF

SEQ ID NO: 266 - protein, 29 aa, Unknown
  Recombinant
QPKLKESGPELKKPGETVKISCKASGYTF

SEQ ID NO: 267 - protein, 29 aa, Unknown
  Recombinant
QQKLKESGPELKKPGETVKISCKASGYTF

SEQ ID NO: 268 - protein, 29 aa, Unknown
  Recombinant
QRKLKESGPELKKPGETVKISCKASGYTF

SEQ ID NO: 269 - protein, 29 aa, Unknown
  Recombinant
QSKLKESGPELKKPGETVKISCKASGYTF

SEQ ID NO: 270 - protein, 29 aa, Unknown
  Recombinant
QTKLKESGPELKKPGETVKISCKASGYTF

SEQ ID NO: 271 - protein, 29 aa, Unknown
  Recombinant
QVKLKESGPELKKPGETVKISCKASGYTF

SEQ ID NO: 272 - protein, 29 aa, Unknown
  Recombinant
QWKLKESGPELKKPGETVKISCKASGYTF

SEQ ID NO: 273 - protein, 29 aa, Unknown
  Recombinant
QYKLKESGPELKKPGETVKISCKASGYTF

SEQ ID NO: 274 - protein, 29 aa, Unknown
  Recombinant
QVKLKESGPELKKPGETVKISCKASGYTF

SEQ ID NO: 275 - protein, 29 aa, Unknown
  Recombinant
QVKLKQSGPELKKPGETVKISCKASGYTF

SEQ ID NO: 276 - protein, 29 aa, Unknown
  Recombinant
QVKLKASGPELKKPGETVKISCKASGYTF

SEQ ID NO: 277 - protein, 29 aa, Unknown
  Recombinant
QVKLKCSGPELKKPGETVKISCKASGYTF

SEQ ID NO: 278 - protein, 29 aa, Unknown
  Recombinant
QVKLKDSGPELKKPGETVKISCKASGYTF

SEQ ID NO: 279 - protein, 29 aa, Unknown
  Recombinant
QVKLKESGPELKKPGETVKISCKASGYTF

SEQ ID NO: 280 - protein, 29 aa, Unknown
  Recombinant
QVKLKFSGPELKKPGETVKISCKASGYTF

SEQ ID NO: 281 - protein, 29 aa, Unknown
  Recombinant
QVKLKGSGPELKKPGETVKISCKASGYTF

SEQ ID NO: 282 - protein, 29 aa, Unknown
  Recombinant
QVKLKHSGPELKKPGETVKISCKASGYTF

SEQ ID NO: 283 - protein, 29 aa, Unknown
  Recombinant
QVKLKISGPELKKPGETVKISCKASGYTF

SEQ ID NO: 284 - protein, 29 aa, Unknown
  Recombinant
QVKLKKSGPELKKPGETVKISCKASGYTF

SEQ ID NO: 285 - protein, 29 aa, Unknown
  Recombinant
QVKLKLSGPELKKPGETVKISCKASGYTF

SEQ ID NO: 286 - protein, 29 aa, Unknown
  Recombinant
QVKLKMSGPELKKPGETVKISCKASGYTF

SEQ ID NO: 287 - protein, 29 aa, Unknown
  Recombinant
QVKLKNSGPELKKPGETVKISCKASGYTF

SEQ ID NO: 288 - protein, 29 aa, Unknown
  Recombinant
QVKLKPSGPELKKPGETVKISCKASGYTF

SEQ ID NO: 289 - protein, 29 aa, Unknown
  Recombinant
QVKLKRSGPELKKPGETVKISCKASGYTF

SEQ ID NO: 290 - protein, 29 aa, Unknown
  Recombinant
QVKLKSSGPELKKPGETVKISCKASGYTF

SEQ ID NO: 291 - protein, 29 aa, Unknown
  Recombinant
QVKLKTSGPELKKPGETVKISCKASGYTF

SEQ ID NO: 292 - protein, 29 aa, Unknown
  Recombinant
QVKLKVSGPELKKPGETVKISCKASGYTF

SEQ ID NO: 293 - protein, 29 aa, Unknown
  Recombinant
QVKLKWSGPELKKPGETVKISCKASGYTF

SEQ ID NO: 294 - protein, 29 aa, Unknown
  Recombinant
QVKLKYSGPELKKPGETVKISCKASGYTF

SEQ ID NO: 295 - protein, 29 aa, Unknown
  Recombinant
QVALKESGPELKKPGETVKISCKASGYTF

SEQ ID NO: 296 - protein, 29 aa, Unknown
  Recombinant
QVCLKESGPELKKPGETVKISCKASGYTF

SEQ ID NO: 297 - protein, 29 aa, Unknown
  Recombinant
QVDLKESGPELKKPGETVKISCKASGYTF

SEQ ID NO: 298 - protein, 29 aa, Unknown
  Recombinant
QVELKESGPELKKPGETVKISCKASGYTF

SEQ ID NO: 299 - protein, 29 aa, Unknown
  Recombinant
QVFLKESGPELKKPGETVKISCKASGYTF

SEQ ID NO: 300 - protein, 29 aa, Unknown
  Recombinant
QVGLKESGPELKKPGETVKISCKASGYTF

SEQ ID NO: 301 - protein, 29 aa, Unknown
  Recombinant
QVHLKESGPELKKPGETVKISCKASGYTF

SEQ ID NO: 302 - protein, 29 aa, Unknown
  Recombinant
QVILKESGPELKKPGETVKISCKASGYTF

SEQ ID NO: 303 - protein, 29 aa, Unknown
  Recombinant
QVKLKESGPELKKPGETVKISCKASGYTF

SEQ ID NO: 304 - protein, 29 aa, Unknown
  Recombinant
QVLLKESGPELKKPGETVKISCKASGYTF

SEQ ID NO: 305 - protein, 29 aa, Unknown
  Recombinant
QVMLKESGPELKKPGETVKISCKASGYTF

SEQ ID NO: 306 - protein, 29 aa, Unknown
  Recombinant
QVNLKESGPELKKPGETVKISCKASGYTF

SEQ ID NO: 307 - protein, 29 aa, Unknown
  Recombinant
QVPLKESGPELKKPGETVKISCKASGYTF

SEQ ID NO: 308 - protein, 29 aa, Unknown
  Recombinant
QVRLKESGPELKKPGETVKISCKASGYTF

SEQ ID NO: 309 - protein, 29 aa, Unknown
  Recombinant
QVSLKESGPELKKPGETVKISCKASGYTF

SEQ ID NO: 310 - protein, 29 aa, Unknown
  Recombinant
QVTLKESGPELKKPGETVKISCKASGYTF

SEQ ID NO: 311 - protein, 29 aa, Unknown
  Recombinant
QVVLKESGPELKKPGETVKISCKASGYTF

SEQ ID NO: 312 - protein, 29 aa, Unknown
  Recombinant
QVWLKESGPELKKPGETVKISCKASGYTF

SEQ ID NO: 313 - protein, 29 aa, Unknown
  Recombinant
QVYLKESGPELKKPGETVKISCKASGYTF

SEQ ID NO: 314 - protein, 29 aa, Unknown
  Recombinant
QVKLAESGPELKKPGETVKISCKASGYTF

SEQ ID NO: 315 - protein, 29 aa, Unknown
  Recombinant
QVKLCESGPELKKPGETVKISCKASGYTF

SEQ ID NO: 316 - protein, 29 aa, Unknown
  Recombinant
QVKLDESGPELKKPGETVKISCKASGYTF

SEQ ID NO: 317 - protein, 29 aa, Unknown
  Recombinant
QVKLEESGPELKKPGETVKISCKASGYTF

SEQ ID NO: 318 - protein, 29 aa, Unknown
  Recombinant
QVKLFESGPELKKPGETVKISCKASGYTF

SEQ ID NO: 319 - protein, 29 aa, Unknown
  Recombinant
QVKLGESGPELKKPGETVKISCKASGYTF

SEQ ID NO: 320 - protein, 29 aa, Unknown
  Recombinant
QVKLHESGPELKKPGETVKISCKASGYTF

SEQ ID NO: 321 - protein, 29 aa, Unknown
  Recombinant
QVKLIESGPELKKPGETVKISCKASGYTF

SEQ ID NO: 322 - protein, 29 aa, Unknown
  Recombinant
QVKLKESGPELKKPGETVKISCKASGYTF

SEQ ID NO: 323 - protein, 29 aa, Unknown
  Recombinant
QVKLLESGPELKKPGETVKISCKASGYTF

SEQ ID NO: 324 - protein, 29 aa, Unknown
  Recombinant
QVKLMESGPELKKPGETVKISCKASGYTF

SEQ ID NO: 325 - protein, 29 aa, Unknown
  Recombinant
QVKLNESGPELKKPGETVKISCKASGYTF

SEQ ID NO: 326 - protein, 29 aa, Unknown
  Recombinant
QVKLPESGPELKKPGETVKISCKASGYTF

SEQ ID NO: 327 - protein, 29 aa, Unknown
  Recombinant
QVKLQESGPELKKPGETVKISCKASGYTF

SEQ ID NO: 328 - protein, 29 aa, Unknown
  Recombinant
QVKLRESGPELKKPGETVKISCKASGYTF

SEQ ID NO: 329 - protein, 29 aa, Unknown
  Recombinant
QVKLSESGPELKKPGETVKISCKASGYTF

SEQ ID NO: 330 - protein, 29 aa, Unknown
  Recombinant
QVKLTESGPELKKPGETVKISCKASGYTF

SEQ ID NO: 331 - protein, 29 aa, Unknown
  Recombinant
QVKLWESGPELKKPGETVKISCKASGYTF

SEQ ID NO: 332 - protein, 29 aa, Unknown
  Recombinant
QVKLYESGPELKKPGETVKISCKASGYTF

SEQ ID NO: 333 - protein, 29 aa, Unknown
  Recombinant
QIQLVQSGPELKKPGETVKISCKASGYTF

SEQ ID NO: 334 - protein, 29 aa, Unknown
  Recombinant
QVQLKESGPELKKPGETVKISCKASGYTF

SEQ ID NO: 335 - protein, 29 aa, Unknown
  Recombinant
QVKLVESGPELKKPGETVKISCKASGYTF

SEQ ID NO: 336 - protein, 29 aa, Unknown
  Recombinant
QIQLKESGPELKKPGETVKISCKASGYTF

SEQ ID NO: 337 - protein, 29 aa, Unknown
  Recombinant
QIKLVESGPELKKPGETVKISCKASGYTF

SEQ ID NO: 338 - protein, 29 aa, Unknown
  Recombinant
QIKLKQSGPELKKPGETVKISCKASGYTF

SEQ ID NO: 339 - protein, 29 aa, Unknown
  Recombinant
QVQLVESGPELKKPGETVKISCKASGYTF

SEQ ID NO: 340 - protein, 29 aa, Unknown
  Recombinant
QVQLKQSGPELKKPGETVKISCKASGYTF

SEQ ID NO: 341 - protein, 29 aa, Unknown
  Recombinant
QVKLVQSGPELKKPGETVKISCKASGYTF

SEQ ID NO: 342 - protein, 29 aa, Unknown
  Recombinant
QIQLVESGPELKKPGETVKISCKASGYTF

SEQ ID NO: 343 - protein, 29 aa, Unknown
  Recombinant
QIQLKQSGPELKKPGETVKISCKASGYTF

SEQ ID NO: 344 - protein, 29 aa, Unknown
  Recombinant
QVQLVQSGPELKKPGETVKISCKASGYTF

SEQ ID NO: 345 - protein, 29 aa, Unknown
  Recombinant
QIKLVQSGPELKKPGETVKISCKASGYTF

SEQ ID NO: 346 - protein, 29 aa, Unknown
  Recombinant
EVQLVESGGGLVQPGGSLRLSCTASGYTF

SEQ ID NO: 347 - protein, 29 aa, Unknown
  Recombinant
QVKLKESGPELKKPGETVKISCKASGYTF

SEQ ID NO: 348 - protein, 29 aa, Unknown
  Recombinant
QVQLVESGGGLVQPGGSLRLSCTASGYTF

SEQ ID NO: 349 - protein, 14 aa, Unknown
  Recombinant
WVKKTPGKGLKWLG

SEQ ID NO: 350 - protein, 14 aa, Unknown
  Recombinant
WVRKAPGKGLEWVG

SEQ ID NO: 351 - protein, 14 aa, Unknown
  Recombinant
WVRKTPGKGLKWVG

SEQ ID NO: 352 - protein, 14 aa, Unknown
  Recombinant
WVKKTPGKGLKWLG

SEQ ID NO: 353 - protein, 31 aa, Unknown
  Recombinant
RFAISLETSARTVYLQINNLRNEDTATYFCF

SEQ ID NO: 354 - protein, 31 aa, Unknown
  Recombinant
RFTISLDTSKNTVYLQMNSLRAEDTATYFCF

SEQ ID NO: 355 - protein, 31 aa, Unknown
  Recombinant
RFTISLDTSKNTVYLQMNSLRAEDTAVYFCA

SEQ ID NO: 356 - protein, 31 aa, Unknown
  Recombinant
RFTISLDTSKNTVYLQMNSLRAEDTAVYYCA

SEQ ID NO: 357 - protein, 31 aa, Unknown
  Recombinant
RFTISRDTSKNTVYLQMNSLRAEDTAVYFCA

SEQ ID NO: 358 - protein, 31 aa, Unknown
  Recombinant
RFTISRDTSKNTVYLQMNSLRAEDTAVYYCA

SEQ ID NO: 359 - protein, 32 aa, Unknown
  Recombinant
RFAISLETSARTVYLQINNLRNEDTATYFCFS

SEQ ID NO: 360 - protein, 10 aa, Unknown
  Recombinant
WGQGTLVTVS

SEQ ID NO: 361 - protein, 11 aa, Unknown
  Recombinant
WGQGTTVTVSS

SEQ ID NO: 362 - protein, 11 aa, Unknown
  Recombinant
WGQGTTATVSS

SEQ ID NO: 363 - protein, 11 aa, Unknown
  Recombinant
WGQGTTCTVSS

SEQ ID NO: 364 - protein, 11 aa, Unknown
  Recombinant
WGQGTTDTVSS

SEQ ID NO: 365 - protein, 11 aa, Unknown
  Recombinant
WGQGTTETVSS

SEQ ID NO: 366 - protein, 11 aa, Unknown
  Recombinant
WGQGTTFTVSS

SEQ ID NO: 367 - protein, 11 aa, Unknown
  Recombinant
WGQGTTGTVSS

SEQ ID NO: 368 - protein, 11 aa, Unknown
  Recombinant
WGQGTTHTVSS

SEQ ID NO: 369 - protein, 11 aa, Unknown
  Recombinant
WGQGTTITVSS

SEQ ID NO: 370 - protein, 11 aa, Unknown
  Recombinant
WGQGTTKTVSS

SEQ ID NO: 371 - protein, 11 aa, Unknown
  Recombinant
WGQGTTMTVSS

SEQ ID NO: 372 - protein, 11 aa, Unknown
  Recombinant
WGQGTTNTVSS

SEQ ID NO: 373 - protein, 11 aa, Unknown
  Recombinant
WGQGTTPTVSS

SEQ ID NO: 374 - protein, 11 aa, Unknown
  Recombinant
WGQGTTQTVSS

SEQ ID NO: 375 - protein, 11 aa, Unknown
  Recombinant
WGQGTTRTVSS

SEQ ID NO: 376 - protein, 11 aa, Unknown
  Recombinant
WGQGTTSTVSS

SEQ ID NO: 377 - protein, 11 aa, Unknown
  Recombinant
WGQGTTTTVSS

SEQ ID NO: 378 - protein, 11 aa, Unknown
  Recombinant
WGQGTTVTVSS

SEQ ID NO: 379 - protein, 11 aa, Unknown
  Recombinant
WGQGTTWTVSS

SEQ ID NO: 380 - protein, 11 aa, Unknown
  Recombinant
WGQGTTYTVSS

SEQ ID NO: 381 - protein, 11 aa, Unknown
  Recombinant
WGQGTTLTVSS

(19) United States
(12) Patent Application Publication
(10) Pub. No.: US 2023/0242666 A1
(43) Pub. Date: Aug. 3, 2023

(54) Methods and Compositions for the Reduction of Chimeric Antigen Receptor Tonic Signaling

(71) Applicants: Cell Medica, Inc., Houston, TX (US); The University of North Carolina at Chapel Hill, Chapel Hill, NC (US)

(72) Inventors: Gianpietro DOTTI, Chapel Hill, NC (US); Elisa LANDONI, Chapel Hill, NC (US); Miriam DROSTE, Houston, TX (US); Abdijapar SHAMSHIEV, Houston, TX (US)

(73) Assignees: Cell Medica, Inc., Houston, TX (US); The University of North Carolina Chapel Hill, Chapel Hill, NC (US)

(21) Appl. No.: 18/003,079

(22) PCT Filed: Jun. 29, 2021
(86) PCT No.: PCT/US2021/039672

§ 371 Date: Dec. 22, 2022

Related U.S. Application Data
(60) Provisional application No. 63/045,646, filed on Jun. 29, 2020.

(51) Int. Cl. C07K 16/30; C07K 14/54

(52) U.S. Cl. CPC C07K 16/3053; C07K 14/5443; C07K 14/5418; C07K 14/5434; C07K 2317/565; C07K 2319/03

(57) ABSTRACT

The present disclosure relates to methods and compositions related to Chimeric Antigen Receptors (“CARs”) and modifications to the framework sequences to eliminate tonic signaling. The compositions include modified binding members having a binding specificity to chondroitin sulfate proteoglycan 4 (CSPG4) and are stable when prepared as single chain antibody (scFv) and incorporated into CARs. The methods further include nucleic acid constructs for the expression of CSPG4 CARs, and the application of the CSPG4 CAR to therapeutic methods for the treatment of cancer. The present disclosure also relates to the modification of humanized framework sequences.

### CROSS-REFERENCE TO RELATED APPLICATIONS

This application is a U.S. National Phase Application of International Application No. PCT/US2021/039672, filed Jun. 29, 2021, which claims the benefit under 35 U.S.C. § 119 of U.S. Provisional Application No. 63/045,646, filed Jun. 29, 2020, each of which is herein incorporated by reference in their entireties, including its respective sequence listing.

### STATEMENT REGARDING FEDERALLY SPONSORED RESEARCH OR DEVELOPMENT

This work was supported by R01 CA193140 (GD), 1R35 GM134864 (NVD), UL1 TR002014 (NVD), RO1DE028172 (SF), RO3CA239193 (SF), and RO3CA216114 (SF) from National Institutes for Health, Passan Foundation (NVD), Department of Defense W81XWH-16-1-0500 (SF) and Il Fondo di Gio Onlus (SP). GD also received support from Cell Medica. The content is solely the responsibility of the authors and does not necessarily represent the official views of the NIH. Sequences of the 763.74 monoclonal antibody and its humanized form have been submitted as provisional patent.

### INCORPORATION OF SEQUENCE LISTING

The Sequence Listing is hereby incorporated by reference in its entirety, including the file named SequenceListing_P35071US01.txt, which is 249,373 bytes in size and was created on Dec. 19, 2022, which is likewise herein incorporated by reference in its entirety.

### BACKGROUND

Chimeric antigen receptors (CARs) in their original conception are fusion proteins in which the variable regions of the heavy chain (V_{H}) and light chain (V_{L}) of a monoclonal antibody are assembled with a non cleavable flexible linker to form a single chain antibody (scFv), which is fused with signaling molecules of the T cell receptor and costimulatory endodomains. See, Eshhar et al., “Specific activation and targeting of cytotoxic lymphocytes through chimeric single chains consisting of antibody-binding domains and the gamma or zeta subunits of the immunoglobulin and T-cell receptors,” Proc. Natl. Acad. Sci. U.S.A 90:720-724 (1993); Finney et al., “Chimeric receptors providing both primary and costimulatory signaling in T cells from a single gene product,” J. Immunol. 161:2791-2797 (1998); Imai et al., “Chimeric receptors with 4-1BB signaling capacity provoke potent cytotoxicity against acute lymphoblastic leukemia,” Leukemia 18:676-684 (2004). Remarkable and sustained antitumor activity is achieved when a CD19-specific CAR is engrafted in T cells and these cells are infused in pediatric patients with acute B-cell leukemia. See Maude et al., “Chimeric antigen receptor T cells for sustained remissions in leukemia,” N. Engl. J. Med. 371:1507-1517 (2014).

CAR engagement with the antigen expressed by tumor cells promotes rapid and profound activation of the T cells which is characterized by cytolysis, cytokine secretion and proliferation. See Dotti et al., “Design and development of therapies using chimeric antigen receptor-expressing T cells,” Immunol. Rev. 257:107-126 (2014). However, it emerged that CARs can cause tonic signaling in T cells, which denotes sustained antigen independent activation leading to rapid T cell exhaustion and impaired antitumor activity (Long et al., “4-1BB costimulation ameliorates T cell exhaustion induced by tonic signaling of chimeric antigen receptors,” Nat. Med. 21:581-590 (2015) (“Long et al. (2015)”). The first report describing tonic signaling in CAR-T cells attributed this effect to the propensity of certain scFvs to self-aggregate causing cell surface CAR clustering and consequent signaling. See id. Similar tonic signaling is observed in other immune cells, including NKT cells. See Xu et al., NKT cells co-expressing a GD2-specific chimeric antigen receptor and IL-15 show enhanced in vivo persistence and antitumor activity against neuroblastoma,” Clin. Cancer Res. 25(23):7126-7138 (2019).

It is well known that V_{H} and V_{L} domains coupled to form scFvs have a tendency to unfold leading to scFv oligomerization. See Worn and Pluckthun, “Stability engineering of antibody single-chain Fv fragments,” J Mol. Biol. 305:989-1010 (2001) (“Worn and Pluckthun (2001)”). A variety of strategies have been developed in the effort to stabilize scFvs that include engineering disulfide bonds between the V_{H} and V_{L} domains, introduction of charged amino acids within the V_{H} and V_{L} domains or grafting the complementarity-determining regions (CDRs) into different framework regions (FWRs). See Young et al., “Thermal stabilization of a single-chain Fv antibody fragment by introduction of a disulphide bond,” FEBS Lett. 377:135-139 (1995); Miller et al., “Stability engineering of scFvs for the development of bispecific and multivalent antibodies,” Protein Eng Des Sel 23:549-557 (2010); and Kugler et al., “Stabilization and humanization of a single-chain Fv antibody fragment specific for human lymphocyte antigen CD19 by designed point mutations and CDR-grafting onto a human framework,” Protein Eng Des Sel 22:135-147 (2009). In the contest of scFvs assembled into a CAR format, the tonic signaling caused by the scFv derived from the murine 14g2a monoclonal antibody was specifically attributed to the FWRs of the antibody. See Long et al. (2015). Remarkably, the engraftment of CDRs of the scFv derived from the FMC63 monoclonal antibody (mAb) into the FWRs of the 14g2a antibody was sufficient to cause tonic signaling of the FMC63 scFv when this scFv was used to generate a CAR indicating that FWRs play a critical role in causing tonic signaling of scFv in the CAR format. See id.

TCR-mediated tonic signaling is a well characterized homeostatic property of naïve T cells and plays a critical role in promoting their long-term persistence. See Myers et al., “Tonic Signals: Why Do Lymphocytes Bother? Trends Immunol. 38:844-857 (2017). However, TCR-mediated tonic signaling requires TCR engagement with self-peptides presented either in Class I or II, and is strictly confined to T cells located in lymphoid organs. See Hochweller et al., “Dendritic cells control T cell tonic signaling required for responsiveness to foreign antigen,” Proc. Natl. Acad. Sci. U.S.A 107:5931-5936 (2010). In sharp contrast, CAR-mediated tonic signaling in T cells, in its strict essence, refers to CAR signaling that is independent from any specific CAR engagement and defined as spontaneous release of cytokines such as IFNγ. See Long et al. (2015). The event triggering CAR-mediated tonic signaling has been identified as the spontaneous aggregation of a sufficient number of CAR molecules, which leads to initiation of signaling. See Long et al., (2015). Here, we confirm that tonic signaling is due to self-aggregation of CAR molecules, and further demonstrate that the CAR-CD3ζ chain is exclusively responsible of the spontaneous cytokine release, since loss of function of the CAR-CD3ζ chain completely abrogates the spontaneous release of INFγ. Spontaneous cytokine release, rather than detection of cell surface markers associated to T cell exhaustion, seems the most robust and reliable assay to define the presence of CAR tonic signaling and reduced functionality.

The instability of synthetic scFvs has long been recognized causing their spontaneous aggregation hindering the generation of soluble reagents. See Worn and Pluckthun (2001); Miller et al., “Stability engineering of scFvs for the development of bispecific and multivalent antibodies,” Protein Eng Des Sel 23:549-557 (2010). We have previously demonstrated that structural modeling and mutagenesis driven by computational protein design can be used to restore specificity of scFvs distorted by fusing V_{L} and V_{H} domains. See Krokhotin et al. “Computationally Guided Design of Single-Chain Variable Fragment Improves Specificity of Chimeric Antigen Receptors,” Mol. Ther. Oncolytics. 15:30-37 (2019). In general, the thermodynamic stability of a protein (ΔG) is crucial for its biological functionality. Amino acid mutations in proteins can disrupt important residue interactions, alter protein active sites, and protein stability. These unstable mutant protein conformations are the underlying sources of numerous human disorders. See Redler et al., “Protein Destabilization as a Common Factor in Diverse Inherited Disorders,” J Mol. Evol. 82:11-16 (2016). Hence, quantifying the effect of mutations on protein structure is important to estimate the protein stability and there by its functionality.

Conflicting data have been reported on the role of the costimulatory CD28 and 4-1BB endodomains in exacerbating or attenuating the CAR tonic signaling in T cells using CARs containing different hinge/spacer regions or different vectors/promoters. See Long et al., (2015); Frigault et al., “Identification of chimeric antigen receptors that mediate constitutive or inducible proliferation of T cells,” Cancer Immunol. Res. 3:356-367 (2015); and Watanabe et al., “Fine-tuning the CAR spacer improves T-cell potency,” Oncoimmunology 5:e1253656 (2016). Despite these conflicting data, it is however possible to conclude that an intrinsic instability of the scFv cannot be corrected by the type of costimulation used or by modifying other components of the CAR structure. Indeed, CAR tonic signaling is frequently considered a good reason to abandon a specific scFv from further CAR development despite the original antibody having an excellent profile in term of antigen specificity and affinity.

### FIELD OF THE DISCLOSURE

The present disclosure relates to engineering binding domains, chimeric antigen receptors, and host cells transformed with engineered binding domains and chimeric antigen receptors to prevent tonic signaling in immune cells. The present disclosure further relates to engineering binding domains, chimeric antigen receptors, and host cells with humanized framework regions containing amino acid substitutions from mouse to provide for human and mouse compatibility.

### SUMMARY OF THE INVENTION

The present disclosure provides for, and includes, a binding member having a binding specificity to chondroitin sulfate proteoglycan 4 (CSPG4) comprising a variable light chain sequence comprising light chain complementarity determining regions (“LCDRs”) LCDR1 to LCDR3 sequences set forth in SEQ ID NOs: 1-3 and a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, a light chain framework sequence 4 (VL-FR4) selected from the group consisting of SEQ ID NOs:250 to 255, and a variable heavy chain sequence comprising heavy chain complementarity determining regions (“HCDRs”) HCDR1 to HCDR3 sequences set forth in SEQ ID NOs: 4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382.

The present disclosure provides for, and includes a nucleic acid encoding a polypeptide comprising a variable light chain sequence comprising light chain complementarity determining regions (“LCDRs”) LCDR1 to LCDR3 sequences set forth in SEQ ID NOs: 1-3 and a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255.

The present disclosure provides for, and includes, a nucleic acid encoding a polypeptide comprising a variable heavy chain sequence comprising heavy chain complementarity determining regions (“HCDRs”) HCDR1 to HCDR3 sequences set forth in SEQ ID NOs: 4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382.

The present disclosure provides for, and includes, a chimeric antigen receptor (CAR) expression construct comprising nucleic acid sequences encoding a chimeric antigen receptor (CAR) coding sequence comprising an ectodomain sequence comprising an antibody or antigen binding fragment thereof that binds to chondroitin sulfate proteoglycan 4 (CSPG4) comprising variable light chain sequence comprising light chain complementarity determining regions (“LCDRs”) LCDR1 to LCDR3 sequences set forth in SEQ ID NOs: 1-3, a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255, a variable heavy chain sequence comprising heavy chain complementarity determining regions (“HCDRs”) HCDR1 to HCDR3 sequences set forth in SEQ ID NOs: 4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382, a transmembrane domain sequence, and an endodomain sequence.

The present disclosure provides for, and includes, a chimeric antigen receptor (CAR) comprising: an ectodomain sequence comprising an antibody or antigen binding fragment thereof that binds to chondroitin sulfate proteoglycan 4 (CSPG4) selected from the group consisting of SEQ ID NOs: 28 to 65, a transmembrane domain sequence, and an endodomain sequence.

The present disclosure provides for, and includes, a nucleic acid molecule encoding a CAR comprising an ectodomain sequence comprising an antibody or antigen binding fragment thereof that binds to chondroitin sulfate proteoglycan 4 (CSPG4) selected from the group consisting of SEQ ID NOs: 28 to 65, a transmembrane domain sequence and an endodomain sequence.

The present disclosure provides for, and includes, a process for the production of an immune cell comprising a CAR comprising isolating peripheral blood mononuclear cells (“PBMCs”) from a donor, separating a natural killer T (NKT) cells, T-cells, or natural killer (NK) cells from the PBMCs to prepare isolated immune cells, and expanding the isolated immune cells for between 1 and 20 days to prepare expanded immune cells for genetic engineering by stimulation of an endogenous T-cell receptor and co-stimulation by costimulatory receptors, cytokines, or a combination of both, and introducing a chimeric antigen receptor (CAR) expression construct comprising an ectodomain sequence comprising an antibody or antigen binding fragment thereof that binds to chondroitin sulfate proteoglycan 4 (CSPG4) comprising a variable light chain selected from the group consisting of SEQ ID NOs:61 to 109 and a variable heavy chain selected from the group consisting of SEQ ID NOs:110 to 152.

The present disclosure provides for, and includes, a genetically engineered immune cell comprising an expression construct encoding a chimeric antigen receptor (CAR) coding sequence comprising an ectodomain sequence comprising an antibody or antigen binding fragment thereof that binds to chondroitin sulfate proteoglycan 4 (CSPG4) comprising a variable light chain sequence comprising light chain complementarity determining regions (“LCDRs”) LCDR1 to LCDR3 sequences set forth in SEQ ID NOs: 1-3, a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255, and a variable heavy chain sequence comprising heavy chain complementarity determining regions (“HCDRs”) HCDR1 to HCDR3 sequences set forth in SEQ ID NOs: 4 to 6, a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382, a transmembrane domain sequence and an endodomain sequence.

The present disclosure provides for, and includes, a population of cells comprising a plurality of genetically engineered immune cells comprising an expression construct encoding a chimeric antigen receptor (CAR) coding sequence comprising an ectodomain sequence comprising an antibody or antigen binding fragment thereof that binds to chondroitin sulfate proteoglycan 4 (CSPG4), the CAR comprising a variable light chain sequence comprising light chain complementarity determining regions (“LCDRs”) LCDR1 to LCDR3 sequences set forth in SEQ ID NOs: 1-3, a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255, and a variable heavy chain sequence comprising heavy chain complementarity determining regions (“HCDRs”) HCDR1 to HCDR3 sequences set forth in SEQ ID NOs: 4 to 6, a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382, a transmembrane domain sequence and an endodomain sequence.

The present disclosure provides for, and includes, a method of inhibiting chondroitin sulfate proteoglycan 4 (CSPG4)-positive cells in an individual, comprising the step of contacting the cells with a therapeutically effective amount of genetically engineered immune cells, wherein the immune cells comprise a chimeric antigen receptor (CAR) comprising an ectodomain sequence comprising an antibody or antigen binding fragment thereof that binds to chondroitin sulfate proteoglycan 4 (CSPG4), the antibody or antigen binding fragment comprising: a variable light chain sequence comprising light chain complementarity determining regions (“LCDRs”) LCDR1 to LCDR3 sequences set forth in SEQ ID NOs: 1-3, a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255, and a variable heavy chain sequence comprising heavy chain complementarity determining regions (“HCDRs”) HCDR1 to HCDR3 sequences set forth in SEQ ID NOs: 4 to 6, a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382, a transmembrane domain sequence and an endodomain sequence.

The present disclosure provides for, and includes, a method for the treatment of cancer, comprising the step of administering to a subject in need thereof the genetically engineered immune cells comprising a chimeric antigen receptor (CAR) that binds to chondroitin sulfate proteoglycan 4 (CSPG4), the CAR comprising an ectodomain sequence comprising a variable light chain sequence comprising light chain complementarity determining regions (“LCDRs”) LCDR1 to LCDR3 sequences set forth in SEQ ID NOs: 1-3, a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255, and a variable heavy chain sequence comprising heavy chain complementarity determining regions (“HCDRs”) HCDR1 to HCDR3 sequences set forth in SEQ ID NOs: 4 to 6, a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382, a transmembrane domain sequence and an endodomain sequence.

The present disclosure provides for, and includes, a kit comprising an a vector, a host cell, or a combination thereof comprising nucleic acid sequences encoding a chimeric antigen receptor (CAR) coding sequence comprising an ectodomain sequence comprising a variable light chain sequence comprising light chain complementarity determining regions (“LCDRs”) LCDR1 to LCDR3 sequences set forth in SEQ ID NOs: 1-3, a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255, and a variable heavy chain sequence comprising heavy chain complementarity determining regions (“HCDRs”) HCDR1 to HCDR3 sequences set forth in SEQ ID NOs: 4 to 6, a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382, a transmembrane domain sequence and an endodomain sequence.

The present disclosure provides for, and includes, a method of maintaining NKT cell expansion potential in NKT cells expressing a chimeric antigen receptor (CAR) coding sequence comprising an ectodomain sequence comprising an antibody or antigen binding fragment thereof that binds to chondroitin sulfate proteoglycan 4 (CSPG4) comprising light chain complementarity determining regions (“LCDRs”) LCDR1 to LCDR3 sequences set forth in SEQ ID NOs: 1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs: 4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence, the method comprising expressing a protein coding sequence comprising a transcriptional activator in the Wnt signaling pathway, and culturing the engineered NKT cells to prepare a population of genetically engineered NKT cells with persistent expansion potential.

A method of reducing tonic signaling in mouse models in an scFv comprising identifying an scFv that has tonic signaling when expressed in a mouse immune cell as part of a CAR, generating as structural model of the scFv, and performing computational mutagenesis to prepare a series of mutagenized scFvs, calculating the free energy of the mutagenized scFvs, aligning the mutagenized scFvs to a humanized scFv comprising framework 1.4 (FW1.4), identifying critical murine residues; and introducing one or more human to mouse residue changes to increase stability of the scFv and prepare a modified humanized scFv for use in mouse models.

### BRIEF DESCRIPTION OF THE DRAWINGS

The present disclosure is disclosed with reference to the accompanying drawings, wherein:

FIG. 1 presents the results of tonic signaling of an embodiment of CAR-T cells expressing the CAR encoding the scFv 763.74(A) (SEQ ID NO:28). FIG. 1A presents representative plots of flow cytometry showing CAR expression in T cells as assessed at day 8 of culture. 763.74(A)CD28 and 763.74(A)4-1BB indicate the specific CAR expressed in T cells. FIG. 1B presents quantification of IFNγ released by T cells expressing control CAR (CTR), 763.74(A)CD28 or 763.74(A)4-1BB CAR without any CAR specific activation. IFNγ is measured in supernatants collected 24 hours after plating 10⁶ cells/well in 24 well plate in 2 mL of complete media without cytokines. Data are presented as mean±SD, n=10, * P=0.0184, *** P=0.0003 (CTR vs. 763.74(A)CD28) P=0.0004 (763.74(A)CD28 vs. 763.74(A)4-1BB) paired t test. FIG. 1C presents quantification of IFNγ released by T cells expressing control CAR (CTR), 763.74(A)CD28ζY6F or 763.74(A)4-1BBζY6F CAR without any CAR specific activation. Data are presented as mean±SD, n=4. FIG. 1D presents representative confocal microscopy images showing GFP aggregation in T cells expressing GFP-tagged CARs (arrows) in which the CARs are obtained using the scFv 763.74(A) and either CD28 or 4-1BB endodomains. Controls CAR (CTR) or GFP-expressing T cells. Shown are representative cells of a single field (Magnification 63×).

FIG. 2 presents an alignment showing Amino acid substitutions in the FWRs of the scFv 763.74(A) that reverse tonic signaling in CAR-T cells. FIG. 2A presents the Sequences of V_{L} (SEQ ID NOs:67) and V_{H} (SEQ ID NOs:110) of the scFv 763.74(A) (SEQ ID NO:28) and scFv 763.74(B) (SEQ ID NOs:68, 115 and 29 respectively). Boxes indicate the locations of the amino acid substitutions. FIG. 2B presents representative flow cytometry plots showing CAR expression in T cells engineered with the scFv 763.74(A) and scFv 763.74(B) CARs encoding either CD28 or 4-1BB endodomains. Non-transduced (NT) T cells are shown as a negative control. FIG. 2C presents the results of the quantification of IFNγ released by T cells expressing the different CARs without any CAR specific activation. CTR indicate control CAR-T cells. IFNγ is measured in supernatants collected 24 hours after plating 10⁶ cells/well in 24 well plate in 2 mL of complete media without cytokines. Data are presented as mean±SD, n=5, *P=0.0154 763.74(A) CD28 vs. 4-1BB; *P=0.194 763.74(A) vs. (B)4-1BB, paired t test. FIG. 2D presents representative confocal microscopy images showing GFP aggregation in T cells expressing GFP-tagged CARs in which the CARs are obtained using either the scFv 763.74(A) or scFv 763.74(B) and either CD28 or 4-1BB endodomains. Shown are representative cells of a single field (Magnification 63×).

FIG. 3 presents amino acid substitutions in the FWRs of the scFv 763.74(A) that destabilize the scFv. FIG. 3A presents a ribbon plot of the structural conformation of the scFv 763.74(B) generated through computational modeling with the FWR mutations shown. FIG. 3B presents select amino acid mutations evaluated for their influence on scFv 763.74(B) stability. Mutations to scFv 763.74(B) structure having ΔΔGₘᵤₜ>0 are destabilizing and subsequently affect spontaneous aggregation of the scFv in the CAR format in T cells. FIG. 3C presents the calculated ΔΔG of mutations at V123 (SEQ ID NOs:256 to 274). FIG. 3D presents the calculated ΔΔG of mutations at E127 (SEQ ID NO:275 to 295).

FIG. 4 presents the results of the effect of T cells expressing the 763.74(B) CAR with CD28 in mediating tumor elimination in a melanoma tumor model. FIG. 4A presents representative flow plots (A) and FIG. 4B presents a summary of the quantification of residual tumor cells of experiments in which control T cells (CTR) and T cells engineered with the scFv 763.74(A) and scFv 763.74(B) CARs encoding either CD28 or 4-1BB endodomains are cocultured with melanoma cell lines (E:T=1:5) for 5 days. Cells are collected and stained with the CD3 and CD276 (B7-H3) mAbs to identify T cells and melanoma cells, respectively, by flow cytometry. Data are presented as mean±SD, n=6, ****P<0.0001, two-way ANOVA with Bonferroni correction. FIG. 4C presents an experimental schema of a melanoma xenograft model. eGFP-FFLuc WM115 (5×10⁵ cells) are injected subcute (s.c.) and 7 days later mice are injected intravenous (i.v.) with control T cells (CTR) or T cells engineered with the scFv 763.74(A) and scFv 763.74(B) CARs encoding either CD28 or 4-1BB endodomains (5×10⁶ cells). FIG. 4D presents representative tumor bioluminescence (BLI) (scale: min=5×10⁶; max=5×10⁸) in mice treated according to scheme (C) FIG. 4E presents a representative graph of tumor volumes in mice engrafted in (D). Dotted lines represent individual mice, and bolded solid lines represent the mean for the group. Summary of 4 independent experiments (n=12 for each condition), ***P=0.00012; ****P<0.0001, two-way ANOVA with Bonferroni correction.

FIG. 5 presents the results of antitumor activity in a glioblastoma tumor model of T cells expressing the 763.74(B) CAR with CD28 FIG. 5A presents the experimental schema of glioblastoma (GBM) xenograft model. GBM-NS (1×10⁵ cells) are injected into the caudate nucleus (i.c.) and 15 days later mice are injected intratumorally with control T cells (CTR) or T cells engineered with the scFv 763.74(A) and scFv 763.74(B) CARs encoding either CD28 or 4-1BB endodomains (2×10⁶ cells). Tumor growth is monitored with magnetic resonance imaging (MRI). FIGS. 5B to 5F present representative MM performed with T1-weighted images (T1-wi) with contrast medium injection and T2-weighted images (T2-wi) showing the pattern of tumor progression and infiltration in mice treated as in (A). FIG. 5G presents Kaplan-Meier survival curves of mice treated as in (A). n=7 mice/group for CTR, 763.74(A)CD28, 763.74(B)4-1BB. n=15 mice/group for 763.74(B)CD28 and 763.74(A)4-1BB. Overall survival statistical analysis is performed using the Mantel-Cox log rank test. FIG. 5H presents a representative time course of CD69 expression in CAR-T cells isolated from the tumor at the indicated time points after intratumor delivery of the T cells. Data are presented as mean±SD, n=2.

FIG. 6 presents the results of the effects of humanization of the FWRs of the scFv 763.74(A) on CAR tonic signaling and anti-tumor activity. FIG. 6A present representative flow cytometry plots showing CAR expression in T cells engineered with h763.74 (#2) (SEQ ID NO:111) and h763.74 (#5) (SEQ ID NO:114) CARs encoding CD28 (h763.74(#2)CD28 and h763.74(#5)CD28) as assessed at day 8 of culture. Non-transduced (NT) T cells are shown as a negative control. FIG. 6B presents a representative quantification of IFNγ in supernatants collected 24 hours of resting control (CTR), h763.74(#2)CD28 and h763.74(#5)CD28 CAR-T cells after plating 5×10⁵ cells/well in 2 mL of medium without cytokines. Data are presented as mean±SD, n=6. FIG. 6C presents representative flow plots of coculture experiments in which control (CTR) or T cells expressing either h763.74(#2)CD28 or h763.74(#5)CD28 CARs are plated with melanoma cell lines (E:T=1:5) for 5 days. Cells are collected and stained with the CD3 and CD276 (B7-H3) mAbs to identify T cells and melanoma cells, respectively, by flow cytometry. FIG. 6D presents an experimental schema of a melanoma xenograft model. eGFP-FFLuc WM115 (5×10⁵ cells) are injected subcute (s.c.) and 7 days later mice are injected intravenous (i.v.) with control T cells (CTR) or T cells engineered with 763.74(B)CD28 CAR or h763.74(#2)CD28 or h763.74(#5)CD28 CARs (5×10⁶ cells). FIG. 6 presents tumor BLI kinetics of mice treated according to scheme (D). Dotted lines represent individual mice, and bolded solid lines represent the mean for the group. Summary of 2 independent experiments (n=10 for each group). FIG. 6F presents quantification of human CD3⁺CD45⁺ cells in the peripheral blood, liver, and spleen at the time of euthanasia of eGFP-FFLuc WM115 tumor-bearing mice treated as in (D). Data are presented as mean±SD, n=6. G. Percentage of CAR-T cells in the peripheral blood, gated on human CD3⁺CD45⁺ cells, at sacrifice in eGFP-FFLuc WM115 tumor-bearing mice treated as in (D). Data are presented as mean±SD, n=6.

FIG. 7 presents constructs and characterization of expression in T cells of CARs generated using the scFv derived from the 763.74 antibody. FIG. 7A presents diagrams of the of 763.74(A) CAR constructs. FIG. 7B presents representative flow plots and summaries of CAR expression in T cells engineered with 763.74(A)CD2ζY6F or 763.74(A)4-1BζY6F CARs assessed at day 8 of culture. Data are presented as mean±SD, n=4. Non-transduced (NT) T cells are shown as a negative control.

FIG. 8 presents the characterization of T cells expressing the scFv 763.74(A) or 763.74(B) CARs in accordance with the present disclosure. FIG. 7A presents a summary of CAR expression in control CAR-T cells (CTR) and T cells engineered with the scFv 763.74(A) and scFv 763.74(B) CARs encoding either CD28 or 4-1BB endodomains as assessed at day 8 of culture. Data are presented as mean±SD, n=8. Non-transduced (NT) T cells are shown as negative control. FIG. 8B presents total cell numbers of CTR and CAR-T cells at days 6 and 10 of a culture as indicated in (A). Data are presented as mean±SD, n=4. FIG. 8C presents microscopic images of representative cells showing the distribution of CAR molecules on the cell surface of T cells expressing GFP-tagged 763.74(A)CD28, 763.74(A)4-1BB, 763.74(B)CD28 and 763.74(B)4-1BB CARs. Shown are representative images of a single field of view taken via confocal microscopy (magnification, 63×). FIG. 8D presents a distribution of GFP-tagged CAR molecules on the cell surface of T cells as in (C) after cross-linking of CARs mediated by an anti-idiotype antibody. Shown are representative images of a single field of view taken via confocal microscopy (magnification, 63×). FIG. 8E presents a graph showing a cell subset composition of CTR and CAR-T cells indicated in (A) at day 10 of culture as assessed by flow cytometry. Data are presented as mean±SD, n=4. FIG. 8F is a representative graph presenting the expression of exhaustion-associated markers in CTR and CAR-T cells indicated in (A) at day 10 of culture as assessed by flow cytometry. Data are presented as mean±SD, n=4.

FIG. 9 presents the evaluation of amino acid mutations for their influence on the stability of the scFv 763.74(B). FIG. 9A presents the change in free energy (ΔΔG) of mutations at position 3 of 763.74(A)V-light (SEQ ID NOs:159 to 1831 at position 3). B. ΔΔG of mutations at T5 (SEQ ID NOs:184 to 202). C. ΔΔG of mutations at A9 (SEQ ID NOs:203 to 221). D. ΔΔG of mutations at E83 (SEQ ID NOs:232 to 249). E. ΔΔG of mutations at Q124 (SEQ ID NOs:256 to 274). F. ΔΔG of mutations at V126 (SEQ ID NOs:315 to 333). G.ΔΔG of mutations at L230 (SEQ ID NOs:276 to 295).

FIG. 10 presents the representative results of Antitumor activity of T cells expressing scFv 763.74(A) and scFv 763.74(B) CARs. FIG. 10A presents the expression of the chondroitin sulfate proteoglycan 4 (CSPG4) antigen in melanoma cell lines assessed by flow cytometry. Dotted and solid lines represent the isotype and CSPG4 mAbs respectively. FIG. 10B presents representative results of the quantification of IFNγ and IL-2 production in the supernatant collected after 24 hours of coculture of control CAR-T cells (CTR) and T cells engineered with scFv 763.74(A) and scFv 763.74(B) CARs encoding either CD28 or 4-1BB endodomains with melanoma cell lines (E:T=1:5). Data are presented as mean±SD, n=5. FIG. 10C presents representative results of the Tumor BLI kinetics of mice engrafted s.c. with eGFP-FFLuc WM115 tumor cells (5×10⁵ cells/mouse) and treated 7 days after with CTR or T cells expressing scFv 763.74(A) and scFv 763.74(B) CARs encoding either CD28 or 4-1BB endodomains (5×10⁶ cells/mouse). Dotted lines represent individual mice, and bolded solid lines represent the mean for the group. Summary of four independent experiments (n=12 for each condition), ****, P<0.0001, two-way ANOVA with Bonferroni's correction.

FIG. 11 presents representative results of T cells expressing the 763.74(B) CAR encoding CD28 showing the elimination of most of the GBM-NS at earliest time points. FIG. 11A is a summary of the quantification of tumor cells at different time points (2, 4, 6 and 24 hours) in representative experiments in which control (CTR) and cells expressing scFv 763.74(A) and scFv 763.74(B) CARs encoding either CD28 or 4-1BB endodomain are cocultured with GBM-NS (E:T=1:5). Data are presented as mean±SD, n=5. FIG. 11B presents representative FSC-SSC dot plots of CAR-T cells after two hours in co-culture with GBM-NS. CAR-T cells and tumor cells are measured by evaluating the percentage of CD45 and CSPG4-expressing cells, respectively. FIG. 11C presents a graph of representative flow cytometry stacked histograms of CD69 expression in CAR-T cells cocultured with GBM-NS.

FIG. 12 presents the humanization process of the scFv 763.74(A) and testing of mutants. FIG. 12A presents the ScFv humanization and engineering work-flow. FIG. 12B presents representative binding results of humanized scFvs h763.74(#2), h763.74(#3), h763.74(#4) and h763.74(#5) analyzed by flow cytometry. CSPG4⁺MDA-MB-231 and CSPG4″ MDA-MB-468 are incubated with the scFvs (10 μg/ml), followed by staining with protein-L-biotin (0.3 μg/ml) and phycoerythrin-labelled streptavidin (SAV-PE). FIG. 12C presents a summary of CAR expression in CTR T cells and in T cells generated using different h763.74 CARs as assessed at day 8 of culture. Data are presented as mean±SD, n=3. Non transduced (NT) T cells are shown as a negative control. FIG. 12D presents a summary of the quantification of residual tumor cells of experiments in which control (CTR) or different types of h763.74 CAR-T cells were cocultured with the WM155 melanoma cell line (E:T=1:5) for 5 days. Data are presented as mean±SD, n=3. Cells are then collected and stained with the CD3 and CD276 (B7-H3) mAbs to identify T cells and melanoma cells, respectively by flow cytometry. FIG. 12E presents the quantification of IFNγ and IL-2 production in the supernatant collected after 24 hours of coculture illustrated in (D) (E:T=1:5). Data are presented as mean±SD, n=5.

FIG. 13 presents the amino acid sequences and representative stability analysis of humanized scFvs. FIG. 13A presents the sequences of the of V_{L} (SEQ ID NO:69 and 72) and V_{H} (SEQ ID NO:72 and 114) chains of humanized scFvs h763.74(#2) (SEQ ID NO:37) and h763.74(#5) (SEQ ID NO:57). Amino acid substitutions (human to murine) are boxed. FIG. 13B presents representative results of the physical stability of humanized scFvs. h763.74(#2) and h763.74(#5) proteins formulated in PBS at lmg/ml and stored at 4° C. and 37° C. for 2 days. The percentage of monomers is analyzed by analytical HPLC.

FIG. 14 presents representative results of antitumor activity of T cells expressing humanized scFv 763.74-based CAR constructs. FIG. 14A present a summary of CAR expression in control T cells (CTR) and in T cells generated using h763.74(#2)CD28 and h763.74(#5)CD28 CARs as assessed at day 8 of culture. Data are presented as mean±SD, n=10. FIG. 14B presents a summary of the quantification of residual tumor cells in experiments in which CTR, h763.74(#2)CD28 and h763.74(#5)CD28 CAR-T cells are cocultured with melanoma cell lines (E:T=1:5) for 5 days. Data are presented as mean±SD, n=4/6. Cells are collected and stained with the CD3 and CD276 (B7-H3) mAbs to identify T cells and melanoma cells, respectively by flow cytometry. FIG. 14C presents representative results of the quantification of IFNγ and IL-2 production in the supernatant collected after 24 hours of the coculture illustrated in (B) (E:T=1:5). Data are presented as mean±SD, n=5. FIG. 14D presents representative results of tumor BLI (color scale: min=5×10⁶; max=5×10⁸) of mice engrafted s.c. with eGFP-FFLuc WM115 tumor cells (5×10⁵ cells/mouse) and treated 7 days after with control CART cells (CTR) and T cells expressing the h763.74(#2)CD28 or h763.74(#5)CD28 CARs (5×10⁶ cells/mouse). FIG. 14E presents a representative quantification of human CD3⁺CD45⁺ cells in the peripheral blood collected at different time points in WM115 tumor-bearing mice treated with CTR, h763.74(#2)CD28 or h763.74(#5)CD28 CAR-T cells (Day 13: n=15, day 25, 31 and 43: n=5). Data are presented as mean±SD, n=5. FIG. 14F presents that percentage of PD-1⁺ cells, gated on human CD3⁺CD45⁺ cells, in the peripheral blood collected at different time points as in (E) (Day 13: n=15, day 25, 31 and 43: n=5) of a representative experiment. Data are presented as mean±SD, n=5. FIG. 14G presents the percentage of PD-1⁺ cells, gated on human CD3⁺CD45⁺ cells, in the peripheral blood, liver, and spleen at sacrifice as in (E) of a representative experiment. Data are presented as mean±SD, n=6-10.

### DETAILED DESCRIPTION

CAR tonic signaling as originally defined, attributes negative effects in T cells and in particular causes poor antitumor effects due to rapid exhaustion. See Long et al. (2015). Our data in vitro and in vivo support this concept. Remarkably, in our xenograft models correction of the stability of the scFv and abrogation of the CAR tonic signaling enhanced significantly the antitumor effects of CAR-T cells encoding the CD28 endodomain that show rapid antitumor effects. This observation is in line with previous work suggesting that 4-1BB show slow antitumor effects that can be mechanistically related to our recent finding that 4-1BB attenuates CAR-CD3γ signaling by recruiting a phosphatase complex in the CAR synapse. See Zhao et al., “Structural Design of Engineered Costimulation Determines Tumor Rejection Kinetics and Persistence of CAR T Cells,” Cancer Cell 28:415-428 (2015); Sun et al., “THEMIS-SHP1 Recruitment by 4-1BB Tunes LCK-Mediated Priming of Chimeric Antigen Receptor-Redirected T Cells. Cancer Cell. 37(2):216-225 (2020) (“Sun et al. 2020”). Furthermore, we would like to suggest that tonic signaling determined as spontaneous release of IFNγ by CAR-T cells due to self-aggregation of the scFv should be considered as a distinct phenomenon from the spontaneous proliferation of CAR-T cells. See Frigault et al., “Identification of chimeric antigen receptors that mediate constitutive or inducible proliferation of T cells,” Cancer Immunol. Res. 3:356-367 (2015). The latter may be instead a positive attribute of the CAR-T cells that may persist in the absence of an immediate antigen stimulation especially in patients with solid tumors in whom tumor cells are not massively accessible in the peripheral blood. Spontaneous proliferation and enhanced survival of CAR-T cells encoding 4-1BB may be due to sustained NF-KB pathway, rather than proximal CAR signaling, and this phenomenon requires further studies to be fully mechanistically defined. See Gomes-Silva et al., “Tonic 4-1BB Costimulation in Chimeric Antigen Receptors Impedes T Cell Survival and Is Vector-Dependent,” Cell Rep. 21:17-26 (2017); Li et al., “4-1BB enhancement of CAR T function requires NF-kappaB and TRAFs,” JCI Insight. 3 (2018); and Philipson et al., “4-1BB costimulation promotes CAR T cell survival through noncanonical NF-kappaB signaling,” Sci. Signal. 13 (2020).

Chimeric antigen receptor (CAR) tonic signaling defined as the eous activation and release of proinflamatory cytokines by T cells genetically grafted with a CAR is considered a negative attribute since it leads to immune cell exhaustion and poor antitumor effects. Unstable murine scFvs cause self-aggregation and murine sequences can induce immune responses in human subjects. Here we demonstrated that the instability of the scFv is critical in causing tonic signaling when the scFv is assembled into the CAR format and the CAR is expressed in T cells. Furthermore, we proved that tonic signaling can be corrected either by the substitution of the amino acids causing instability within the murine FWRs of the scFv or humanization of the FWRs. Correction of the tonic signaling enhances the antitumor effects of the CAR-T cells.

Here, we report that CAR tonic signaling is caused by the intrinsic instability of the monoclonal antibody single chain Fv that promotes self-aggregation and signaling via CD3γ chain included into the CAR. This phenomenon is detected in CAR encoding either CD28 or 4-1BB costimulatory endodomains. Instability of the monoclonal antibody single chain Fv is caused by specific amino acids within the framework regions that can be identified by computational modeling. Substitutions of the amino acids causing instability or humanization of the framework regions correct tonic signaling of the CAR without causing modification of the antigen specificity and enhance the antitumor effects of CAR-T cells.

Here we demonstrate that an unstable scFv causes self-aggregation of CAR molecules leading to CAR tonic signaling in T cells. The CSPG4-specific CAR we have generated uses a murine scFv from the 763.74 mAb. This scFv expressed in E. coli showed a tendency to aggregation and the scFv was not producible in a soluble form.

Here we show that the instability of the scFv is the exclusive cause of self-aggregation and tonic signaling of the CAR since modeling driven specific amino acid substitutions within the FWRs that stabilize the scFv abrogate the CAR tonic signaling. The results presented below demonstrate through structural and functional analysis that the instability of the scFv causes CAR self-aggregation and tonic signaling. Amino acid substitutions or humanization of the FWRs abrogate the tonic signaling and enhance the functionality of CAR-T cells.

Employing computational mutagenesis, facilitated by Eris tool to delineate the effect of FWR mutations on the structure of the scFv 763.74(A). Computational modeling demonstrates how amino acid substitutions within the FWRs affect the stability of the scFvs indicating that computational analyses can be implemented to assess and correct the stability of scFv without affecting their specificity. Finally, we demonstrate that substituting the murine FWRs with a stable human FWRs such as the framework rFW1.4 also corrected CAR tonic signaling without modifying the antigen specificity. See Borras et al., “Generic approach for the generation of stable humanized single-chain Fv fragments from rabbit monoclonal antibodies,” J Biol. Chem. 285:9054-9066 (2010). The stable human framework rFW1.4 can accommodate CDRs of different origin and allows analyses of soluble scFv proteins to assess their physicochemical stability. This approach thus can be used to select scFvs with optimal physical and binding properties to design CARs. Taken together, the data suggest that stable and monomeric scFvs are applicable to generate CARs that avoid antigen-independent tonic signaling. ScFvs engineered with the human framework rFW1.4 may also have a low likelihood for immunogenicity and could potentially be repeatedly infused to the patients with full efficacy.

Our data indicate that using CARs in which the only difference is represented by the intracytoplasmic tail of the costimulatory endodomain, both CD28 and 4-1BB caused tonic signaling.

Importantly, stabilization of the scFv abrogated tonic signaling regardless of the costimulation used within the CAR indicating that an unstable scFv can be rescued by either mutations or humanization of the FWRs and that both CD28 and 4-1BB costimulation can be used.

Here we show that amino acid substitutions into the FWRs can stabilize the scFv and correct the tonic signaling of the CAR. We additionally show that substitution of the murine FWRs with stable human FWRs also prevents antigen-independent activation of CAR-T cells. Thus, we localize the cause of CAR tonic signaling to the unstable scFvs used for the CAR construction and provide structure-based strategies to correct tonic signaling.

Overall, we demonstrate that tonic signaling of a CAR is due to self-aggregation of an unstable scFv and that it can be abrogated by stabilization of the FWRs of scFv obtained by amino acid substitutions or humanization. Correction of the scFv stability and elimination of CAR tonic signaling enhance the antitumor effects of CAR-T cells.

The present disclosure provides for, and includes, binding members having binding specificity to chondroitin sulfate proteoglycan 4 (CSPG4) (Gene ID: 1464 (human) and 121021 (mouse)).

As used herein, a binding member normally comprises an antibody VH and a VL domain. VH domains of specific binding members are also provided for use in the invention. Within each of the VH and VL domains are complementarity determining regions, (“CDRs”, “HCDRs” and “LCDRs” respectively), and framework regions, (“FRs”, “VH-FRs” and “VL-FRs” respectively). A VH domain comprises HCDR1 to HCDR3, and a VL domain comprises a LCDR1 to LCDR3. An antibody molecule may comprise an antibody VH domain comprising a VH CDR1, CDR2 and CDR3 and framework sequences VH-FR1 to VH-FR4. It may alternatively or also comprise an antibody VL domain comprising a VL CDR1, CDR2 and CDR3 and a framework sequences VL-FR1 to VL-FR4. All VH and VL sequences, CDR sequences, sets of CDRs and sets of HCDRs and sets of LCDRs disclosed herein represent embodiments of a specific binding member. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. As used herein, framework sequences can be obtained from any source including but not limited to mouse, human, rabbit, and rat. In aspects, the framework sequences are modified to comprise sequences from two species. In aspects, the framework sequences are modified human framework sequences with one or more modifications to introduce the mouse residue to increase stability. Such modified humanized frameworks enable the use of the same construct in both mouse and human cells.

As used herein, the binding members comprise light chain complementarity determining regions (“LCDRs”) LCDR1 to LCDR3 sequences as set forth in SEQ ID NOs:1-3 and heavy chain complementarity determining regions (“HCDRs”) HCDR1 to HCDR3 sequences as set forth in SEQ ID NOs:4 to 6.

The present disclosure provides for, and includes, binding members having binding specificity to chondroitin sulfate proteoglycan 4 (CSPG4) and comprise the framework sequences. In aspects, the light chain frameworks sequences (“VL-FR”) are selected from the group consisting of SEQ ID NOs:153 to 255, where light chain framework sequence 1 (VL-FR1) is selected from the group consisting of SEQ ID NOs:153 to 221, light chain framework sequence 2 (VL-FR2) is selected from the group consisting of SEQ ID NOs:222 to 225, light chain framework sequence 3 (VL-FR3) is selected from the group consisting of SEQ ID NOs:226 to 249, light chain framework sequence 4 (VL-FR4) is selected from the group consisting of SEQ ID NOs:250 to 255. In aspects, the heavy chain framework sequences (“VH-FR”) are selected from the group consisting of SEQ ID NOs:256 to 382, where heavy chain framework sequence 1 (VH-FR1) is selected from the group consisting of SEQ ID NOs:256 to 349, heavy chain framework sequence 2 (VH-FR2) is selected from the group consisting of SEQ ID NOs:350 to 353, heavy chain framework sequence 3 (VH-FR3) is selected from the group consisting of SEQ ID NOs:354 to 360, and heavy chain framework sequence 4 (VH-FR4) is selected from the group consisting of SEQ ID NOs:361 to 382.

The present disclosure provides for, and includes, a binding member having a binding specificity to CSPG4 wherein light chain framework sequence 1 (VL-FR1) is selected from the group consisting of SEQ ID NOs:153 to 158, light chain framework sequence 2 (VL-FR2) is selected from the group consisting of SEQ ID NOs:222 to 225, light chain framework sequence 3 (VL-FR3) is selected from the group consisting of SEQ ID NOs:226 to 231, light chain framework sequence 4 (VL-FR4) is selected from the group consisting of SEQ ID NOs:250 to 255, heavy chain framework sequence 1 (VH-FR1) is selected from the group consisting of SEQ ID NOs:347 to 349, heavy chain framework sequence 2 (VH-FR2) is selected from the group consisting of SEQ ID NOs:350 to 353, heavy chain framework sequence 3 (VH-FR3) is selected from the group consisting of SEQ ID NOs:354 to 360, and heavy chain framework sequence 4 (VH-FR4) is selected from the group consisting of SEQ ID NOs:361 to 362. In aspects, the variable light chain sequence is selected from the group consisting of SEQ ID NOs:69 to 72 and the variable heavy chain sequence selected from the group consisting of SEQ ID NOs:111 to 114. In an aspect, the variable light chain sequence is SEQ ID NO:69 and the variable heavy chain sequence is SEQ ID NO:111. In an aspect, the variable light chain sequence is SEQ ID NO:70 and the variable heavy chain sequence is SEQ ID NO:112. In an aspect, the variable light chain sequence is SEQ ID NO:71 and the variable heavy chain sequence is SEQ ID NO:113. In an aspect, the variable light chain sequence is SEQ ID NO:71 and the variable heavy chain sequence is SEQ ID NO:114.

The present disclosure provides for, and includes, nucleic acid sequences encoding polypeptides having a binding specificity to CSPG4 wherein the variable light chain sequence selected from the group consisting of SEQ ID NOs:67 to 109 and a variable heavy chain sequence selected from the group consisting of SEQ ID NOs:110 to 152. In aspects, the variable light chain sequence is selected from the group consisting of SEQ ID NOs:69 to 72 and the variable heavy chain sequence selected from the group consisting of SEQ ID NOs:111 to 114. In an aspect, the variable light chain sequence is SEQ ID NO:69 and the variable heavy chain sequence is SEQ ID NO:111. In an aspect, the variable light chain sequence is SEQ ID NO:70 and the variable heavy chain sequence is SEQ ID NO:112. In an aspect, the variable light chain sequence is SEQ ID NO:71 and the variable heavy chain sequence is SEQ ID NO:113. In an aspect, the variable light chain sequence is SEQ ID NO:71 and the variable heavy chain sequence is SEQ ID NO:114.

In aspects, the nucleic acid sequence encodes polypeptides comprising a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249 and a light chain framework sequence 4 (VL-FR4) selected from the group consisting of SEQ ID NOs:250 to 255. In an aspect, the variable light chain sequence is SEQ ID NO:69. In another aspect, the variable light chain sequence is SEQ ID NO:70. In an aspect, the variable light chain sequence is SEQ ID NO:71. In an aspect, the variable light chain sequence is SEQ ID NO:72. In another aspect, the variable light chain sequence is SEQ ID NO:73. In an aspect, the variable light chain sequence is SEQ ID NO:74. In an aspect, the variable light chain sequence is SEQ ID NO:76. In another aspect, the variable light chain sequence is SEQ ID NO:77. In an aspect, the variable light chain sequence is SEQ ID NO:78. In an aspect, the variable light chain sequence is SEQ ID NO:79. In another aspect, the variable light chain sequence is SEQ ID NO:80. In an aspect, the variable light chain sequence is SEQ ID NO:83. In an aspect, the variable light chain sequence is SEQ ID NO:96. In another aspect, the variable light chain sequence is SEQ ID NO:97. In an aspect, the variable light chain sequence is SEQ ID NO:102. In an aspect, the variable light chain sequence is SEQ ID NO:103. In another aspect, the variable light chain sequence is SEQ ID NO:104. In an aspect, the variable light chain sequence is SEQ ID NO:105. In an aspect, the variable light chain sequence is SEQ ID NO:106. In another aspect, the variable light chain sequence is SEQ ID NO:107. In aspects, the nucleic acid sequences encoding a variable light chain sequence can be combined with a nucleic acid sequence encoding a heavy chain sequence selected from the group consisting of SEQ ID NOs:110 to 152.

In aspects, the nucleic acid sequence encodes polypeptides comprising a heavy chain sequence comprising heaving chain complementarity determining regions HCDR1 to HCDR3 sequences set forth in SEQ ID NOs:4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382. In an aspect, the variable heavy chain sequence is SEQ ID NO:111. In another aspect, the variable heavy chain sequence is SEQ ID NO:112. In an aspect, the variable heavy chain sequence is SEQ ID NO:113. In an aspect, the variable heavy chain sequence is SEQ ID NO:114. In another aspect, the variable heavy chain sequence is SEQ ID NO:115. In an aspect, the variable heavy chain sequence is SEQ ID NO:116. In an aspect, the variable heavy chain sequence is SEQ ID NO:117. In another aspect, the variable heavy chain sequence is SEQ ID NO:118. In an aspect, the variable heavy chain sequence is SEQ ID NO:119. In an aspect, the variable heavy chain sequence is SEQ ID NO:120. In another aspect, the variable heavy chain sequence is SEQ ID NO:121. In an aspect, the variable heavy chain sequence is SEQ ID NO:122. In an aspect, the variable heavy chain sequence is SEQ ID NO:123. In another aspect, the variable heavy chain sequence is SEQ ID NO:135. In an aspect, the variable heavy chain sequence is SEQ ID NO:136. In an aspect, the variable heavy chain sequence is SEQ ID NO:137. In another aspect, the variable heavy chain sequence is SEQ ID NO:138. In an aspect, the variable heavy chain sequence is SEQ ID NO:145. In aspects, the nucleic acid sequences encoding a variable heavy chain sequence can be combined with a nucleic acid sequence encoding a light chain sequence selected from the group consisting of SEQ ID NOs:67 to 109.

The present disclosure provides for, and includes, chimeric antigen receptors (“CARs”) and expression constructs for CARs having a binding specificity to chondroitin sulfate proteoglycan 4 (CSPG4). As provided herein the CDR regions are as set forth above.

The term “chimeric antigen receptor” or “CAR,” as used herein, refers to an artificial T cell receptor that is engineered to be expressed on an immune effector cell and specifically bind an antigen. In aspects, CARs comprise and ectodomain, a transmembrane domain, and an endodomain. In aspects, an additional “spacer” or “hinge region” is included in the CAR. In certain aspects, a CAR can comprise an ectodomain and transmembrane domain without an endodomain, but more CARs of the present application include the endodomain and provide for intracellular signaling.

As used herein, the term “ectodomain” refers to the extracellular portion of a CAR and encompasses a signal peptide, an antigen recognition domain (e.g., binding member), and a spacer or hinge region that links the antigen recognition domain to the transmembrane domain. When expressed, the signal peptide may be removed, typically using the endogenous cellular pathways.

As used herein, an “antigen recognition domain” generally comprises a single chain variable fragment (scFv) specific for a particular cancer antigen. In some aspects, where there are two or more CARs in the same cell, the second CAR may comprise an scFv specific for another particular antigen.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. A suitable, but non limiting example is SEQ ID NO:11. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Suitable V_{H} and V_{L} encoding sequences are identified above.

As used herein, a “spacer” or “hinge region” is an optional linker portion of the recombinant protein further that is a short peptide fragment between the transmembrane domain and the antibody recognition domain. The spacer or hinge region can be between 1 and 20 amino acids. Examples of hinge regions for the ectodomain include the CH2CH3 region of immunoglobulin, the hinge region from IgG1, and portions of CD3.

As used herein, a “transmembrane domain” is a region of predominantly nonpolar amino acid residues that when the protein is expressed, traverses the bilayer at least once. Generally, the transmembrane domain is encoded by 18 to 21 amino acid residues and adopts an alpha helical configuration. As used herein, the transmembrane domain may be of any kind known in the art. In aspects the transmembrane domain is selected from the group consisting of CD28 (Gene ID:940, 12487), CD3-ζ (Gene ID:919; 12503 CD247), CD4 (Gene ID:920, 12504), CD8 (Gene ID:924, 12525), CD16 (Gene ID:2214; 14131; Fcgr3), NKp44 (Gene ID:9436, NCR2), NKp46 (Gene ID:9437, 17086, NCR1), and NKG2d (Gene ID:22914; 27007 KLRK1). In aspects the transmembrane domain is the CD28 (Gene ID:940, 12487) transmembrane domain.

As used herein, the term “endodomain” refers to the intracellular domain of a CAR that provides for signal transmission in a cell. Generally, the endodomain can be further divided into two parts, a stimulatory domain and optionally, a co-stimulatory domain. In aspects, the endodomain sequence is selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12). The most commonly used endodomain component is CD3-zeta that contains 3 ITAMs and that transmits an activation signal to the NKT cell after the antigen is bound. Another commonly used endodomain is the TNF receptor superfamily member 9 (Gene ID 3604, e.g., 4-1BB or CD137) endodomain. Other suitable stimulatory domains can be obtained from 2B4 (CD244), TNF receptor superfamily member 9 (Gene ID 3604, e.g., 4-1BB or CD137), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

The present disclosure provides for, and includes, nucleic acid expression constructs encoding chimeric antigen receptors (“CARs”) and CAR proteins produced therefrom, having a binding specificity to chondroitin sulfate proteoglycan 4 (CSPG4) comprising an ectodomain, a transmembrane domain sequence and an endodomain sequence. In aspects, the ectodomain variable light chain sequence comprises LCDR1 to LCDR3 sequences set forth in SEQ ID NOs: 1-3 and HCDR1 to HCDR3 set forth in SEQ ID NOs:4 to 6. As provided herein, ectodomain comprises light chain framework sequence 1 (VL-FR1) selected from the group consisting of SEQ ID NOs:153 to 221, light chain framework sequence 2 (VL-FR2) selected from the group consisting of SEQ ID NOs:222 to 225, light chain framework sequence 3 (VL-FR3) selected from the group consisting of SEQ ID NOs:226 to 249, and light chain framework sequence 4 (VL-FR4) selected from the group consisting of SEQ ID NOs:250 to 255. As provided herein, ectodomain comprises heavy chain framework sequence 1 (VH-FR1) selected from the group consisting of SEQ ID NOs:256 to 349, heavy chain framework sequence 2 (VH-FR2) selected from the group consisting of SEQ ID NOs:350 to 353, heavy chain framework sequence 3 (VH-FR3) selected from the group consisting of SEQ ID NOs:354 to 360, and heavy chain framework sequence 4 (VH-FR4) selected from the group consisting of SEQ ID NOs:361 to 382. The CARs of the present disclosure include a variable light chain sequence selected from the group consisting of SEQ ID NOs:67 to 109 and a variable heavy chain sequence selected from the group consisting of SEQ ID NOs:110 to 152. In aspects, the ectodomain comprises an scFv polypeptide sequence selected from the group consisting of SEQ ID NOs:28 to 66. In an aspect, the ectodomain comprises an scFv polypeptide sequence of SEQ ID NO:28. In an aspect, the ectodomain comprises an scFv polypeptide sequence of SEQ ID NO:34. In an aspect, the ectodomain comprises an scFv polypeptide sequence of SEQ ID NO:46. In an aspect, the ectodomain comprises an scFv polypeptide sequence of SEQ ID NO:47. In an aspect, the ectodomain comprises an scFv polypeptide sequence of SEQ ID NO:57.

The nucleic acid expression constructs encoding chimeric antigen receptors (“CARs”), and CAR proteins produced therefrom, having a binding specificity to chondroitin sulfate proteoglycan 4 (CSPG4) comprising an ectodomain, a transmembrane domain sequence and an endodomain sequence comprise transmembrane domains selected from the group consisting of CD28 (Gene ID:940, 12487), CD3-ζ (Gene ID:919; 12503 CD247), CD4 (Gene ID:920, 12504), CD8 (Gene ID:924, 12525), CD16 (Gene ID:2214; 14131; Fcgr3), NKp44 (Gene ID:9436, NCR2), NKp46 (Gene ID:9437, 17086, NCR1), and NKG2d (Gene ID:22914; 27007 KLRK1). In an aspect the transmembrane domain is the CD28 transmembrane domain and the ectodomain is selected from the scFv sequences selected from the group consisting of SEQ ID NOs:28 to 66. In an aspect the transmembrane domain is the CD3-ζ transmembrane domain and the ectodomain is selected from the scFv sequences selected from the group consisting of SEQ ID NOs:28 to 66. In an aspect the transmembrane domain is the CD4 transmembrane domain and the ectodomain is selected from the scFv sequences selected from the group consisting of SEQ ID NOs:28 to 66. In an aspect the transmembrane domain is the CD8 transmembrane domain and the ectodomain is selected from the scFv sequences selected from the group consisting of SEQ ID NOs:28 to 66. In an aspect the transmembrane domain is the CD16 transmembrane domain and the ectodomain is selected from the scFv sequences selected from the group consisting of SEQ ID NOs:28 to 66. In an aspect the transmembrane domain is the NKp44 transmembrane domain and the ectodomain is selected from the scFv sequences selected from the group consisting of SEQ ID NOs:28 to 66. In an aspect the transmembrane domain is the NKp46 transmembrane domain and the ectodomain is selected from the scFv sequences selected from the group consisting of SEQ ID NOs:28 to 66. In an aspect the transmembrane domain is the NKG2d transmembrane domain and the ectodomain is selected from the scFv sequences selected from the group consisting of SEQ ID NOs:28 to 66. The ectodomain and transmembrane domains may be combined with any one of the endodomain sequences selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

The nucleic acid expression constructs encoding chimeric antigen receptors (“CARs”), and CAR proteins produced therefrom, having a binding specificity to chondroitin sulfate proteoglycan 4 (CSPG4) comprising an ectodomain, a transmembrane domain sequence and an endodomain sequence comprise an endodomain sequence selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

In an aspect the endodomain is the CD 28 endodomain, the transmembrane domain is the CD28 transmembrane domain and the ectodomain is selected from the scFv sequences selected from the group consisting of SEQ ID NOs:28 to 66. In an aspect the endodomain is the 4-1BB endodomain, the transmembrane domain is the CD28 transmembrane domain and the ectodomain is selected from the scFv sequences selected from the group consisting of SEQ ID NOs:28 to 66. In an aspect the endodomain is the CD247 endodomain, the transmembrane domain is the CD28 transmembrane domain and the ectodomain is selected from the scFv sequences selected from the group consisting of SEQ ID NOs:28 to 66. In an aspect the endodomain is the 2B4 endodomain, the transmembrane domain is the CD28 transmembrane domain and the ectodomain is selected from the scFv sequences selected from the group consisting of SEQ ID NOs:28 to 66. In an aspect the endodomain is the IL-21 endodomain, the transmembrane domain is the CD28 transmembrane domain and the ectodomain is selected from the scFv sequences selected from the group consisting of SEQ ID NOs:28 to 66. In an aspect the endodomain is the HCST endodomain, the transmembrane domain is the CD28 transmembrane domain and the ectodomain is selected from the scFv sequences selected from the group consisting of SEQ ID NOs:28 to 66. In an aspect the endodomain is the TYROBP endodomain, the transmembrane domain is the CD28 transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the CD3-ζ transmembrane domain and the ectodomain is selected from the scFv sequences selected from the group consisting of SEQ ID NOs:28 to 66. In an aspect the endodomain is the 4-1BB endodomain, the transmembrane domain is the CD3-ζ transmembrane domain and the ectodomain is selected from the scFv sequences selected from the group consisting of SEQ ID NOs:28 to 66. In an aspect the endodomain is the CD247 endodomain, the transmembrane domain is the CD3-transmembrane domain and the ectodomain is selected from the scFv sequences selected from the group consisting of SEQ ID NOs:28 to 66. In an aspect the endodomain is the CD3-ζ endodomain, the transmembrane domain is the CD3-ζ transmembrane domain and the ectodomain is selected from the scFv sequences selected from the group consisting of SEQ ID NOs:28 to 66. In an aspect the endodomain is the IL-21 endodomain, the transmembrane domain is the CD3-transmembrane domain and the ectodomain is selected from the scFv sequences selected from the group consisting of SEQ ID NOs:28 to 66. In an aspect the endodomain is the HCST endodomain, the transmembrane domain is the CD3-ζ transmembrane domain and the ectodomain is selected from the scFv sequences selected from the group consisting of SEQ ID NOs:28 to 66. In an aspect the endodomain is the TYROBP endodomain, the transmembrane domain is the CD3-ζ transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the CD4 transmembrane domain and the ectodomain is selected from the scFv sequences selected from the group consisting of SEQ ID NOs:28 to 66. In an aspect the endodomain is the 4-1BB endodomain, the transmembrane domain is the CD4 transmembrane domain and the ectodomain is selected from the scFv sequences selected from the group consisting of SEQ ID NOs:28 to 66. In an aspect the endodomain is the CD247 endodomain, the transmembrane domain is the CD4 transmembrane domain and the ectodomain is selected from the scFv sequences selected from the group consisting of SEQ ID NOs:28 to 66. In an aspect the endodomain is the CD4 endodomain, the transmembrane domain is the CD4 transmembrane domain and the ectodomain is selected from the scFv sequences selected from the group consisting of SEQ ID NOs:28 to 66. In an aspect the endodomain is the IL-21 endodomain, the transmembrane domain is the CD4 transmembrane domain and the ectodomain is selected from the scFv sequences selected from the group consisting of SEQ ID NOs:28 to 66. In an aspect the endodomain is the HCST endodomain, the transmembrane domain is the CD4 transmembrane domain and the ectodomain is selected from the scFv sequences selected from the group consisting of SEQ ID NOs:28 to 66. In an aspect the endodomain is the TYROBP endodomain, the transmembrane domain is the CD4 transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the CD8 transmembrane domain and the ectodomain is selected from the scFv sequences selected from the group consisting of SEQ ID NOs:28 to 66. In an aspect the endodomain is the 4-1BB endodomain, the transmembrane domain is the CD8 transmembrane domain and the ectodomain is selected from the scFv sequences selected from the group consisting of SEQ ID NOs:28 to 66. In an aspect the endodomain is the CD247 endodomain, the transmembrane domain is the CD8 transmembrane domain and the ectodomain is selected from the scFv sequences selected from the group consisting of SEQ ID NOs:28 to 66. In an aspect the endodomain is the CD4 endodomain, the transmembrane domain is the CD8 transmembrane domain and the ectodomain is selected from the scFv sequences selected from the group consisting of SEQ ID NOs:28 to 66. In an aspect the endodomain is the IL-21 endodomain, the transmembrane domain is the CD8 transmembrane domain and the ectodomain is selected from the scFv sequences selected from the group consisting of SEQ ID NOs:28 to 66. In an aspect the endodomain is the HCST endodomain, the transmembrane domain is the CD8 transmembrane domain and the ectodomain is selected from the scFv sequences selected from the group consisting of SEQ ID NOs:28 to 66. In an aspect the endodomain is the TYROBP endodomain, the transmembrane domain is the CD8 transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the CD16 transmembrane domain and the ectodomain is selected from the scFv sequences selected from the group consisting of SEQ ID NOs:28 to 66. In an aspect the endodomain is the 4-1BB endodomain, the transmembrane domain is the CD16 transmembrane domain and the ectodomain is selected from the scFv sequences selected from the group consisting of SEQ ID NOs:28 to 66. In an aspect the endodomain is the CD247 endodomain, the transmembrane domain is the CD16 transmembrane domain and the ectodomain is selected from the scFv sequences selected from the group consisting of SEQ ID NOs:28 to 66. In an aspect the endodomain is the CD4 endodomain, the transmembrane domain is the CD16 transmembrane domain and the ectodomain is selected from the scFv sequences selected from the group consisting of SEQ ID NOs:28 to 66. In an aspect the endodomain is the IL-21 endodomain, the transmembrane domain is the CD16 transmembrane domain and the ectodomain is selected from the scFv sequences selected from the group consisting of SEQ ID NOs:28 to 66. In an aspect the endodomain is the HCST endodomain, the transmembrane domain is the CD16 transmembrane domain and the ectodomain is selected from the scFv sequences selected from the group consisting of SEQ ID NOs:28 to 66. In an aspect the endodomain is the TYROBP endodomain, the transmembrane domain is the CD16 transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the NKp44 transmembrane domain and the ectodomain is selected from the scFv sequences selected from the group consisting of SEQ ID NOs:28 to 66. In an aspect the endodomain is the 4-1BB endodomain, the transmembrane domain is the NKp44 transmembrane domain and the ectodomain is selected from the scFv sequences selected from the group consisting of SEQ ID NOs:28 to 66. In an aspect the endodomain is the CD247 endodomain, the transmembrane domain is the NKp44 transmembrane domain and the ectodomain is selected from the scFv sequences selected from the group consisting of SEQ ID NOs:28 to 66. In an aspect the endodomain is the CD4 endodomain, the transmembrane domain is the NKp44 transmembrane domain and the ectodomain is selected from the scFv sequences selected from the group consisting of SEQ ID NOs:28 to 66. In an aspect the endodomain is the IL-21 endodomain, the transmembrane domain is the NKp44 transmembrane domain and the ectodomain is selected from the scFv sequences selected from the group consisting of SEQ ID NOs:28 to 66. In an aspect the endodomain is the HCST endodomain, the transmembrane domain is the NKp44 transmembrane domain and the ectodomain is selected from the scFv sequences selected from the group consisting of SEQ ID NOs:28 to 66. In an aspect the endodomain is the TYROBP endodomain, the transmembrane domain is the NKp44 transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the NKp46 transmembrane domain and the ectodomain is selected from the scFv sequences selected from the group consisting of SEQ ID NOs:28 to 66. In an aspect the endodomain is the 4-1BB endodomain, the transmembrane domain is the NKp46 transmembrane domain and the ectodomain is selected from the scFv sequences selected from the group consisting of SEQ ID NOs:28 to 66. In an aspect the endodomain is the CD247 endodomain, the transmembrane domain is the NKp44 transmembrane domain and the ectodomain is selected from the scFv sequences selected from the group consisting of SEQ ID NOs:28 to 66. In an aspect the endodomain is the CD4 endodomain, the transmembrane domain is the NKp46 transmembrane domain and the ectodomain is selected from the scFv sequences selected from the group consisting of SEQ ID NOs:28 to 66. In an aspect the endodomain is the IL-21 endodomain, the transmembrane domain is the NKp46 transmembrane domain and the ectodomain is selected from the scFv sequences selected from the group consisting of SEQ ID NOs:28 to 66. In an aspect the endodomain is the HCST endodomain, the transmembrane domain is the NKp46 transmembrane domain and the ectodomain is selected from the scFv sequences selected from the group consisting of SEQ ID NOs:28 to 66. In an aspect the endodomain is the TYROBP endodomain, the transmembrane domain is the NKp46 transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

In an aspect the endodomain is the CD 28 endodomain, the transmembrane domain is the NKG2d transmembrane domain and the ectodomain is selected from the scFv sequences selected from the group consisting of SEQ ID NOs:28 to 66. In an aspect the endodomain is the 4-1BB endodomain, the transmembrane domain is the NKG2d transmembrane domain and the ectodomain is selected from the scFv sequences selected from the group consisting of SEQ ID NOs:28 to 66. In an aspect the endodomain is the CD247 endodomain, the transmembrane domain is the NKG2d transmembrane domain and the ectodomain is selected from the scFv sequences selected from the group consisting of SEQ ID NOs:28 to 66. In an aspect the endodomain is the CD4 endodomain, the transmembrane domain is the NKG2d transmembrane domain and the ectodomain is selected from the scFv sequences selected from the group consisting of SEQ ID NOs:28 to 66. In an aspect the endodomain is the IL-21 endodomain, the transmembrane domain is the NKG2d transmembrane domain and the ectodomain is selected from the scFv sequences selected from the group consisting of SEQ ID NOs:28 to 66. In an aspect the endodomain is the HCST endodomain, the transmembrane domain is the NKG2d transmembrane domain and the ectodomain is selected from the scFv sequences selected from the group consisting of SEQ ID NOs:28 to 66. In an aspect the endodomain is the TYROBP endodomain, the transmembrane domain is the NKG2d transmembrane domain and the ectodomain is selected from the scFv sequences selected from the group consisting of SEQ ID NOs:28 to 66. In aspects, the nucleic acid expression constructs encoding CARs further encode at least one protein coding sequence for a growth factor, a protein coding sequence for a transcriptional activator in the Wnt signaling pathway, or both, wherein each are separated from the CAR coding sequences by a foot-and-mouth disease virus (FMDV) 2A sequence or a FMDV 2A related cis acting hydrolase element (CHYSEL) sequence.

The present disclosure provides for, and includes nucleic acid expression constructs encoding chimeric antigen receptors as provided above that further comprise a protein coding sequence for a transcriptional activator in the Wnt signaling pathway. Also provided for, and included, are expression constructs that encodes a polyprotein comprising a CAR, a protein sequence for a transcriptional activator in the Wnt signaling pathway and up to three additional protein coding sequences. In an aspect, the protein sequences for a transcriptional activator in the Wnt signaling pathway and up to three additional protein coding sequences are separated by an autonomous intra-ribosomal self-processing peptide. In an aspect, the autonomous intra-ribosomal self-processing is a foot-and-mouth disease virus (FMDV) 2A sequence or a related cis acting hydrolase element (CHYSEL). In aspects, the transcriptional activator in the Wnt signaling pathway is selected from the group consisting of lymphoid enhancer binding factor 1 (LEF1, Gene ID 51176), beta-catenin (CTNNB1, Gene ID 1499), Smad3 (Gene ID 4088), HNF1 homeobox A (HNF1A, Gene ID: 6927 (alt. TCF1)), transcription factor 7 (TCF7, Gene ID:6932 (alt. TCF1)) and TLE family member 1, transcriptional corepressor (TLE 1, Gene ID 7088)).

In an aspect, the expression construct comprises an ectodomain selected from the scFv sequences selected from the group consisting of SEQ ID NOs:28 to 66, a transmembrane domain selected from the group consisting of CD28, CD3-ζ, CD4, CD8, CD16, NKp44, NKp46, and NKG2d, and an endodomain selected from the group consisting of CD28, 4-1BB, CD3-ζ, 2B4, Interleukin 21, HCST, and TYROBP and further comprising a protein coding sequence for a transcriptional activator in the Wnt signaling pathway selected from the group consisting of lymphoid enhancer binding factor 1, beta-catenin, Smad3, HNF1 homeobox A, transcription factor 7 and TLE family member 1, transcriptional corepressor. In an aspect, the expression construct comprises an scFv ectodomain sequence selected from the group consisting of SEQ ID NOs:28 to 66, the CD28 transmembrane domain, the CD28 endodomain and LEF1 separated by an autonomous intra-ribosomal self-processing peptide, wherein LEF1 is selected from the group consisting of Reference Sequence (RefSeq) ID NOs: NP_057353.1, NP_001124185.1, and NP_001124186.1. In an aspect, the expression construct comprises an scFv ectodomain sequence selected from the group consisting of SEQ ID NOs:28 to 66, the CD3-ζ transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD28 endodomain and LEF1 separated by an autonomous intra-ribosomal self-processing peptide. In an aspect, the expression construct comprises an scFv ectodomain sequence selected from the group consisting of SEQ ID NOs:28 to 66, the CD28 transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the CD4 transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the CD8 transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the CD16 transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the 4-1BB endodomain and LEF1 separated by an autonomous intra-ribosomal self-processing peptide. In an aspect, the expression construct comprises an scFv ectodomain sequence selected from the group consisting of SEQ ID NOs:28 to 66, the CD28 transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD3-endodomain and LEF1 separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD3-endodomain and LEF1 separated by an autonomous intra-ribosomal self-processing peptide.

The present disclosure provides for, and includes, the expression constructs described above comprising an ectodomain selected from the scFv sequences selected from the group consisting of SEQ ID NOs:28 to 66, a transmembrane domain selected from the group consisting of CD28, CD3-ζ, CD4, CD8, CD16, NKp44, NKp46, and NKG2d, and an endodomain selected from the group consisting of CD28, 4-1BB, CD3-ζ, 2B4, Interleukin 21, HCST, and TYROBP and further comprising a protein coding sequence for a transcriptional activator in the Wnt signaling pathway, and further comprising at least one protein coding sequence for a growth factor selected from the group consisting of interleukin-15 (IL-15), interleukin-7 (IL-7), interleukin-12 (IL-12), interleukin-18 (IL-18), interleukin-21 (IL-21), interleukin-27 (IL-27), interleukin-33 (IL-33), and combinations thereof. In an aspect, the expression construct comprises an scFv ectodomain sequence selected from the group consisting of SEQ ID NOs:28 to 66, the CD28 transmembrane domain, the CD28 endodomain, and LEF1 separated by an autonomous intra-ribosomal self-processing peptide, wherein LEF1 is selected from the group consisting of Reference Sequence (RefSeq) ID NOs: NP_057353.1, NP_001124185.1, and NP_001124186.1, and IL-15, each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD28 endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD28 transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD28 transmembrane domain, the CD3-ζ endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the CD3-endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD3-ζ endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD3-ζ endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD3-t endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD3-ζ endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD3-ζ endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD3-ζ endodomain and LEF1 and IL-15 each separated by an autonomous intra-ribosomal self-processing peptide.

In an aspect, the expression construct comprises an scFv ectodomain sequence selected from the group consisting of SEQ ID NOs:28 to 66, the CD28 transmembrane domain, the CD28 endodomain, and LEF1 separated by an autonomous intra-ribosomal self-processing peptide, wherein LEF1 is selected from the group consisting of Reference Sequence (RefSeq) ID NOs: NP_057353.1, NP_001124185.1, and NP_001124186.1, and IL-21, each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD28 endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD28 transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the 4-1BB endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD28 transmembrane domain, the CD3-ζ endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the CD3-ζ endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD3-ζ endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD3-ζ endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD3-ζ endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD3-t endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD3-ζ endodomain and LEF1 and IL-21 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD3-ζ endodomain and LEF1 and IL-21 each separated by an autonomous intra-ribosomal self-processing peptide.

In an aspect, the expression construct comprises an scFv ectodomain sequence selected from the group consisting of SEQ ID NOs:28 to 66, the CD3-ζ transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD28 endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD28 transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the 4-1BB endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the 4-1BB endodomain and LEF1 and IL-15 each separated by an autonomous intra-ribosomal self-processing peptide. In an aspect, the expression construct comprises an scFv ectodomain sequence selected from the group consisting of SEQ ID NOs:28 to 66, the CD28 transmembrane domain, the CD3-endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD3-ζ transmembrane domain, the CD3-ζ endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD4 transmembrane domain, the CD3-ζ endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD8 transmembrane domain, the CD3-ζ endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the CD16 transmembrane domain, the CD3-ζ endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKp44 transmembrane domain, the CD3-ζ endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKp46 transmembrane domain, the CD3-ζ endodomain and LEF1, IL-15, and IL-21 each separated by an autonomous intra-ribosomal self-processing peptide. In an aspect, the expression construct comprises an scFv ectodomain sequence selected from the group consisting of SEQ ID NOs:28 to 66, the NKG2d transmembrane domain, the CD3-endodomain and LEF1, IL-15, and IL-21 each separated by an autonomous intra-ribosomal self-processing peptide.

Also included and provided for by the present disclosure are expression constructs encoding a CAR as provided above and further comprising a DNA sequence encoding a small hairpin RNA (shRNA) sequence targeting an MHC class I or MHC class II gene, wherein the shRNA sequence is embedded in an artificial microRNA (amiR) scaffold.

The present disclosure includes and provides for chimeric antigen receptors comprising an ectodomain sequence comprising an antibody or antigen binding fragment thereof that binds to chondroitin sulfate proteoglycan 4 (CSPG4) selected from the group consisting of SEQ ID NOs:28 to 65, a transmembrane domain sequence; and an endodomain sequence, each as described above for the nucleic acid expression constructs. In an aspect, the ectodomain sequence is selected from the group consisting of SEQ ID NOs:34, 46, 47, and 57. In another aspect, the ectodomain sequence comprises SEQ ID NO:34. In another aspect, the ectodomain sequence comprises SEQ ID NO:46. In another aspect, the ectodomain sequence comprises SEQ ID NO:47. In another aspect, the ectodomain sequence comprises SEQ ID NO:57. Suitable combinations of ectodomains, transmembrane domains and endodomains are recited above. In an aspect, the present disclosure provides for an ectodomain sequence selected from the group consisting of SEQ ID NOs:28 to 65, in combination with a CD28 transmembrane domain and a CD28 endodomain. In an aspect, the present disclosure provides for an ectodomain sequence selected from the group consisting of SEQ ID NOs:28 to 65, in combination with a CD28 transmembrane domain and a CD3-ζ endodomain. In an aspect, the present disclosure provides for an ectodomain sequence selected from the group consisting of SEQ ID NOs:28 to 65, in combination with a CD28 transmembrane domain and a 4-1BB endodomain. In an aspect, the present disclosure provides for an ectodomain sequence selected from the group consisting of SEQ ID NOs:28 to 65, in combination with a CD3-ζ transmembrane domain and a CD28 endodomain. In an aspect, the present disclosure provides for an ectodomain sequence selected from the group consisting of SEQ ID NOs:28 to 65, in combination with a CD3-ζ transmembrane domain and a CD3-ζ endodomain. In an aspect, the present disclosure provides for an ectodomain sequence selected from the group consisting of SEQ ID NOs:28 to 65, in combination with a CD3-ζ transmembrane domain and a 4-1BB endodomain. In an aspect, the present disclosure provides for an ectodomain sequence selected from the group consisting of SEQ ID NOs:28 to 65, in combination with a CD4 transmembrane domain and a CD28 endodomain. In an aspect, the present disclosure provides for an ectodomain sequence selected from the group consisting of SEQ ID NOs:28 to 65, in combination with a CD4 transmembrane domain and a CD3-ζ endodomain. In an aspect, the present disclosure provides for an ectodomain sequence selected from the group consisting of SEQ ID NOs:28 to 65, in combination with a CD4 transmembrane domain and a 4-1BB endodomain.

The present specification further provides, and includes, host cells transformed or transfected with the expression constructs and nucleic acid sequences as defined above. Preferably, the host cells are genetically engineered to introduce exogenous nucleic acid sequences that are transcribed and translated to express one or more proteins. Introducing exogenous nucleic acid sequences can be performed using methods known in the art including transformation, transfection and transduction. In an aspect, the host cell is a bacteria. In other aspects, the hose cell is an immune cell selected from a natural killer T (NKT) cell, a T-cell, and a natural killer (NK) cell. In an aspect, the host cell is a T-cell. In another aspect, the host cell is an NKT cell. In a further aspect, the host cell is a Type-I NKT cell. In yet a further aspect, the host cell is a CD62L positive Type I NKT cells. As provided herein, the host cells are part of a population of host cells.

The present specification provides for, and includes, a population of cells comprising a plurality of genetically engineered immune cells comprising an expression construct encoding a chimeric antigen receptor (CAR) coding sequence comprising an ectodomain sequence comprising an antibody or antigen binding fragment thereof that binds to chondroitin sulfate proteoglycan 4 (CSPG4) as described above. Specific combinations of expression constructs are described above and hereby incorporated by reference.

In aspects of the present disclosure, the population of cells comprise genetically engineered immune cells comprise a natural killer T (NKT) cells, T-cells, or natural killer (NK) cells. In an aspect, the genetically engineered immune cells in the population comprises a plurality of CD62L-positive Type I NKT cells. In an aspect, the population comprises plurality of CD62L-positive Type I NKT cells comprising at least 50% of said plurality of cells.

The present disclosure provides for, and includes, a process for the production of immune cells comprising that expression constructs for the expression of a CAR as described above in detail. As provided herein, the process comprises isolating PBMCs from a donor, separating natural killer T (NKT) cells, T-cells, or natural killer (NK) cells from the PBMCs to prepare isolated immune cells, expanding the isolated immune cells for between 1 and 20 days to prepare expanded immune cells for genetic engineering by stimulation of an endogenous T-cell receptor and co-stimulation by costimulatory receptors, cytokines, or a combination of both, and introducing a chimeric antigen receptor (CAR) expression construct comprising an ectodomain sequence comprising an antibody or antigen binding fragment thereof that binds to chondroitin sulfate proteoglycan 4 (CSPG4) comprising a variable light chain selected from the group consisting of SEQ ID NOs:61 to 109 and a variable heavy chain selected from the group consisting of SEQ ID NOs:110 to 152. In an aspect, the steps of preparing immune cells for genetic engineering further comprises separating the immune cells from PBMCs using anti T-cell, anti-NK cell, or anti-NKT microbeads. In an aspect, the stimulation comprises culturing with growth factors selected from IL-7, IL-12, IL-15, IL-21, TNF-alpha, or a combination thereof. In an aspect, the immune cell is an NKT cell. In a further aspect, the NKT cell is a Type I NKT cell and the step of expanding the separated NKT cells comprises culturing the I NKT cell in the presence of at least aGalCer, and a growth factor for at least 1 day to prepare NKT cells for genetic engineering.

In an aspect, the culture period for expanding the isolated immune cells for between 1 and 20 days to prepare expanded immune cells for genetic engineering is for 1 to 2 days, 1 to 3 days, 1 to 4 days, 1 to 5 days, 1 to 6 days, 1 to 7 days, 1 to 8 days, 1 to 9 days, 1 to 10 days, 1 to 11 days, 1 to 12 days, 1 to 13 days, 1 to 14 days, 1 to 15 days, 1 to 16 days, 1 to 17 days, 1 to 18 days, 1 to 19 days, 1 to 20 days, 2 to 3 days, 2 to 4 days, 2 to 5 days, 2 to 6 days, 2 to 7 days, 2 to 8 days, 2 to 9 days, 2 to 10 days, 2 to 11 days, 2 to 12 days, 2 to 13 days, 2 to 14 days, 2 to 15 days, 2 to 16 days, 2 to 17 days, 2 to 18 days, 2 to 19 days, 2 to 20 days, 3 to 4 days, 3 to 5 days, 3 to 6 days, 3 to 7 days, 3 to 8 days, 3 to 9 days, 3 to 10 days, 3 to 11 days, 3 to 12 days, 3 to 13 days, 3 to 14 days, 3 to 15 days, 3 to 16 days, 3 to 17 days, 3 to 18 days, 3 to 19 days, 3 to 20 days, 4 to 5 days, 4 to 6, 4 to 7, 4 to 8, 4 to 9, 4 to 10, 4 to 11, 4 to 12, 4 to 13 days, 4 to 14 days, 4 to 15 days, 4 to 16 days, 4 to 17 days, 4 to 18 days, 4 to 19 days, 4 to 20 days, 5 to 6 days, 5 to 7 days, 5 to 8 days, 5 to 9 days, 5 to 10 days, 5 to 11 days, 5 to 12 days, 5 to 13 days, 5 to 14 days, 5 to 15 days, 5 to 16 days, 5 to 17 days, 5 to 18 days, 5 to 19 days, 5 to 20 days, 6 to 7 days, 6 to 8 days, 6 to 9 days, 6 to 10 days, 6 to 11 days, 6 to 12 days, 6 to 13 days, 6 to 14 days, 6 to 15 days, 6 to 16 days, 6 to 17 days, 6 to 18 days, 6 to 19 days, 6 to 20 days, 7 to 8 days, 7 to 9 days, 7 to 10 days, 7 to 11 days, 7 to 12 days, 7 to 13, 7 to 14, 7 to 15, 7 to 16, 7 to 17, 7 to 18, 7 to 19 days, 7 to 20 days, 8 to 9 days, 8 to 10 days, 8 to 11 days, 8 to 12 days, 8 to 13 days, 8 to 14 days, 8 to 15, days 8 to 16 days, 8 to 17 days, 8 to 18 days, 8 to 19 days, 8 to 20 days, 9 to 10 days, 9 to 11 days, 9 to 12 days, 9 to 13 days, 9 to 14 days, 9 to 15, days 9 to 16 days, 9 to 17 days, 9 to 18 days, 9 to 19 days, 9 to 20 days, 10 to 11 days, 10 to 12 days, 10 to 13 days, 10 to 14 days, 10 to 15 days, 10 to 16 days, 10 to 17 days, 10 to 18 days, 10 to 19 days, 10 to 20 days, 11 to 12 days, 11 to 13 days, 11 to 14 days, 11 to 15 days, 11 to 16 days, 11 to 17 days, 11 to 18 days, 11 to 19 days, 11 to 20 days, 12 to 13 days, 12 to 14 days, 12 to 15 days, 12 to 16 days, 12 to 17 days, 12 to 18 days, 12 to 19 days, 12 to 20 days, 13 to 14 days, 13 to 15 days, 13 to 16 days, 13 to 17 days, 13 to 18 days, 13 to 19 days, 13 to 20 days, 14 to 15 days, 14 to 16 days, 14 to 17 days, 14 to 18 days, 14 to 19 days, 14 to 20 days, 15 to 16 days, 15 to 17 days, 15 to 18 days, 15 to 19 days, 15 to 20 days, 16 to 17 days, 16 to 18 days, 16 to 19 days, 16 to 20 days, 17 to 18 days, 17 to 19 days, 17 to 20 days, 18 to 19 days, 18 to 20 days, or 19 to 20 days to prepare a population of genetically engineered NKT cells for genetic engineering. In an aspect, the isolated immune cells for use in a process for the production of immune cells comprising that expression constructs for the expression of a CAR are T cells. In another aspect, the isolated immune cells are NK cells. In an aspect, the isolated immune cells are Type I NKT cells. In a further aspect, expanded immune cells are CD62L positive Type I NKT cells. As provided herein, the process for the production of immune cells includes isolating immune cells that are Type I NKT cells and expanding the Type I NKT cells by culturing in the presence of at least aGalCer, IL-2, and IL-21. As provided above, the expression constructs may further include expression of an exogenous growth factor. In another aspect, the expression constructs include a transcriptional activator in the Wnt signaling pathway. Also included and provided for is the addition of a DNA sequence encoding a small hairpin RNA (shRNA) sequence targeting an MHC class I or MHC class II gene, wherein the shRNA sequence is embedded in an artificial microRNA (amiR) scaffold. In aspects, protein coding sequences can be separated by a foot-and-mouth disease virus (FMDV) 2A sequence or a FMDV 2A related cis acting hydrolase element (CHYSEL) sequence.

The present disclosure provides for, and includes, methods for inhibiting chondroitin sulfate proteoglycan 4 (CSPG4) positive cells in an individual, comprising the step of contacting the cells with a therapeutically effective amount of genetically engineered immune cells, wherein said immune cells comprise a chimeric antigen receptor (CAR) comprising a transmembrane domain sequence, an endodomain sequence and an ectodomain sequence comprising an antibody or antigen binding fragment thereof that binds to chondroitin sulfate proteoglycan 4 (CSPG4), said antibody or antigen binding fragment comprising: a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255 and a variable heavy chain sequence comprising heavy chain complementarity determining regions (“LCDRs”) HCDR1 to HCDR3 sequences set forth in SEQ ID NOs:4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382. As used herein, inhibiting chondroitin sulfate proteoglycan 4 (CSPG4) positive cells in an individual comprises inhibiting proliferation, inhibiting activity, or a combination of both.

In aspects according to the present disclosure, the methods for inhibiting chondroitin sulfate proteoglycan 4 (CSPG4) positive cells in an individual comprises genetically engineered immune cells are that are natural killer T (NKT) cells, T-cells, or natural killer (NK) cells. In an aspect, the genetically engineered immune cells are T-cells. In another aspect, genetically engineered immune cells are NKT cells. In yet another aspect, the NKT cells are Type-I NKT cells. In a further aspect, the Type-I NKT cells are CD62L-positive Type-I NKT cells. In aspects, the genetically engineered Type-I NKT cells comprise a majority of said genetically engineered immune cells. In a further aspect, the genetically engineered Type-I NKT cells comprise a majority of CD62L-positive Type-I NKT cells.

The present disclosure provides for, and includes, a method for the treatment of cancer, comprising the step of administering to a subject in need thereof the genetically engineered immune cells comprising a chimeric antigen receptor (CAR) that binds to chondroitin sulfate proteoglycan 4 (CSPG4) comprising a transmembrane domain sequence, an endodomain sequence and an ectodomain sequence comprising an antibody or antigen binding fragment thereof that binds to chondroitin sulfate proteoglycan 4 (CSPG4), said antibody or antigen binding fragment comprising: a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255 and a variable heavy chain sequence comprising heavy chain complementarity determining regions (“LCDRs”) HCDR1 to HCDR3 sequences set forth in SEQ ID NOs:4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382.

As provided herein the methods of treating cancers expressing CPSPG4. In an aspect, the methods provide for treating a cancer selected from the group consisting of melanoma, metastatic melanoma disease glioblastoma, anaplastic thyroid cancer, soft tissue sarcoma, glioma, and leukemia. In an aspect, the cancer for treatment using the methods of the present disclosure is a metastatic melanoma disease selected from superficial spreading melanoma, lentigo maligna, lentigo maligna melanoma, acral lentiginous melanoma, or nodular melanoma. In an aspect, the methods provide for treating a soft tissue sarcoma selected from a Leiomyosarcoma, Dedifferentiated Liposarcoma, Undifferentiated pleomorphic sarcoma (UPS), Malignant Fibrous Histiocytoma, High-Grade Spindle Cell Sarcoma, Myxofibrosarcoma, Malignant Peripheral Nerve Sheath Tumor (MPNST), and Synovial Sarcoma. See PMID: 32900797. In an aspect, the cancer for treatment using the disclosed methods is a glioblastoma. See PMID:34113233. In another aspect, the methods provide for treating anaplastic thyroid cancer. See PMID:34078123. The method according to claim [0091], wherein said cancer is a glioma, astrocytoma, or oligodendroglioma. See PMID:32599896. In an aspect, the cancer for treatment using the disclosed methods is leukemia including but not limited to B-cell precursor leukemia and MLL-translocated leukemia. See PMID:31195686.

The present disclosure provides for, and includes, treating cancers expressing CPSPG4 by administering to a subject in need thereof a therapeutic amount of the genetically engineered immune cells disclosed herein. In an aspect, the genetically engineered immune cells are natural killer T (NKT) cells, T-cells, or natural killer (NK) cells. In an aspect, the genetically engineered immune cells are T-cells. In another aspect, the genetically engineered immune cells are NKT cells. In a further aspect, the genetically engineered NKT cells are Type-I NKT cells. In yet a further aspect, the genetically engineered Type-I NKT cells are CD62L-positive Type-I NKT cells. In certain aspects, the genetically engineered Type-I NKT cells comprise a majority of said genetically engineered immune cells. In certain aspects, the genetically engineered Type-I NKT cells comprise a majority of said genetically engineered CD62L-positive Type-I NKT cells

The present disclosure provides for, and includes, kits comprising the expression vectors, host cells, or combinations thereof as described above. In an aspect the kits comprise vectors or cells having a nucleic acid sequence encoding a transmembrane domain sequence, an endodomain sequence and an ectodomain sequence comprising an antibody or antigen binding fragment thereof that binds to chondroitin sulfate proteoglycan 4 (CSPG4), said antibody or antigen binding fragment comprising: a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221, a light chain framework sequence 2 (VL-FR2) selected from the group consisting of SEQ ID NOs:222 to 225, a light chain framework sequence 3 (VL-FR3) selected from the group consisting of SEQ ID NOs:226 to 249, and a light chain framework sequence 4 (VL-FR4) selected from the group consisting of SEQ ID NOs:250 to 255 and a variable heavy chain sequence comprising heavy chain complementarity determining regions (“LCDRs”) HCDR1 to HCDR3 sequences set forth in SEQ ID NOs:4 to 6 and a heavy chain framework sequence 1 (VH-FR1) selected from the group consisting of SEQ ID NOs:256 to 349, a heavy chain framework sequence 2 (VH-FR2) selected from the group consisting of SEQ ID NOs:350 to 353, a heavy chain framework sequence 3 (VH-FR3) selected from the group consisting of SEQ ID NOs:354 to 360, and a heavy chain framework sequence 4 (VH-FR4) selected from the group consisting of SEQ ID NOs:361 to 382.

The present disclosure provides for, and includes, a method for maintaining NKT cell expansion potential in NKT cells expressing a chimeric antigen receptor (CAR) coding sequence comprising an ectodomain sequence comprising an antibody or antigen binding fragment thereof that binds to chondroitin sulfate proteoglycan 4 (CSPG4) comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence, said method comprising method comprising expressing a protein coding sequence comprising a transcriptional activator in the Wnt signaling pathway, and culturing said engineered NKT cells to prepare a population of genetically engineered NKT cells with persistent expansion potential.

In certain aspects, the population of genetically engineered NKT cells with persistent expansion potential according to the present disclosure comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the total cell population. In certain aspects, the population of genetically engineered NKT cells with persistent expansion potential according to the present disclosure comprises at least 10% up to 80%, between 10% and 90%, between 10% and 95%, between 10% and 98%, between 10% and 99%, and up to 100% wherein non-engineered NKT cells comprise less than 99.9% of the total population. In aspects, the population of genetically engineered NKT cells with persistent expansion potential according to the present disclosure comprises at least 50%, at least 55%, at least 60%, at least 65%, at least 70%, at least 75%, at least 80%, at least 85%, at least 90%, or at least 95% of the total cell population. In certain aspects, the population of genetically engineered NKT cells with persistent expansion potential according to the present disclosure comprises between at least 50% up to 70%, between 50% up to 80%, between 50% and 90%, between 50% and 95%, between 50% and 98%, between 50% and 99%, and up to 100% wherein non-engineered NKT cells comprise less than 99.9% of the total population. In aspects, the engineered NKT cells further express a CAR. In other aspects, the engineered NKT cells express a CAR and an exogenous growth factor.

In certain aspects, the population of genetically engineered NKT cells with persistent expansion potential according to the present disclosure comprises at least 50%, at least 55%, at least 60%, at least 65%, at least 70%, at least 75%, at least 80%, at least 85%, at least 90%, or at least 95% CD62L(+) NKT cells. In certain aspects, the population of genetically engineered NKT cells with persistent expansion potential according to the present disclosure comprises 50% to 55%, 50% to 60%, about 50% to 65%, 50% to 70%, 50% to 75%, 50% to 80%, 50% to 85%, 50% to 90%, 50% to 95%, 50% to 100%, 55% to 60%, 55% to 65%, 55% to 70%, 55% to 75%, 55% to 80%, 55% to 85%, 55% to 90%, 55% to 95%, 55% to 100%, 60% to 65%, 60% to 70%, 60% to 75%, 60% to 80%, 60% to 85%, 60% to 90%, 60% to 95%, 60% to 100%, 65% to 70%, 65% to 75%, 65% to 80%, 65% to 85%, 65% to 90%, 65% to 95%, 65% to 100%, 70% to 75%, 70% to 80%, 70% to 85%, 70% to 90%, 70% to 95%, 70% to 100%, 75% to 80%, 75% to 85%, 75% to 90%, 75% to 95%, 75% to 100%, 80% to 85%, 80% to 90%, 80% to 95%, 80% to 100%, 85% to 90%, 85% to 95%, 85% to 100%, 90% to 95%, 90% to 100%, or 95% to 100% CD62L(+) NKT cells. In certain aspects, the population of genetically engineered NKT cells with persistent expansion potential according to the present disclosure comprises 50%, 55%, 60%, 65%, 70%, 75%, 80%, 85%, 90%, 95%, or 100% CD62L(+) NKT cells. In aspects, the engineered CD62L(+) NKT cells further express a CAR. In other aspects, the engineered CD62L(+) NKT cells express a CAR and an exogenous growth factor.

The present disclosure provides for, and includes, methods for maintaining NKT cell expansion potential comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential, wherein said engineered NKT cells are cultured for a period of time.

In aspects, the methods for maintaining NKT cell expansion potential comprising the steps of engineering NKT cells further comprises separating desired cells from the population of genetically engineered NKT cells with persistent expansion potential. In an aspect, the method further comprises separating the engineered NKT cells by the expression of CD62L to produce a selected population of CD62L(+) genetically engineered NKT cells. In an aspect, the method further comprises separating the engineered NKT cells by the expression of 4-1BB to produce a selected population of 4-1BB (+) genetically engineered NKT cells.

The methods for maintaining NKT cell expansion potential of the present disclosure provides for, and includes, populations of genetically engineered NKT cells with persistent expansion potential exhibit in vivo persistence as infiltrates into neuroblastoma xenografts in humanized NSG mice.

The present disclosure provides for, and includes, methods for reducing NKT cell exhaustion comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% CD62L(+) NKT cells of the total population of cells. In aspects, the 10% or greater population of genetically engineered CD62L(+) NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered CD62L(+) NKT cells further express a CAR. In other aspects, the engineered CD62L(+) NKT cells express a CAR and an exogenous growth factor.

The present disclosure provides for, and includes, a method of reducing tonic signaling in mouse models in an scFv comprising: identifying an scFv that has tonic signaling when expressed in a mouse immune cell as part of a CAR; generating a structural model of said scFv, and performing computational mutagenesis to prepare a series of mutagenized scFvs; calculating the free energy of said mutagenized scFvs; aligning said mutagenized scFvs to a humanized scFv comprising framework 1.4 (FW1.4) identifying critical murine residues; and introducing one or more human to mouse residue changes to increase stability of said scFv and prepare a modified humanized scFv for use in mouse models. In an aspect, the method comprises a modified humanize FW1.4 comprising at least one of a light chain framework regions (VL-FR) 1 to 4 of SEQ ID NOs:7 to 10, a linker region of SEQ ID NO:11, or heavy chain framework region (VH-FR) 1 to 4 of SEQ ID NOs:12 to 15. In an aspect, the modified humanized scFv comprises modified a modified FR regions selected from the group consisting of SEQ ID NOs:16 to 27. As used herein, tonic signaling is measured by the spontaneous release of cytokines such as INFγ. In aspects according to the present disclosure, the methods of reducing tonic signaling in mouse models in an scFv as part of a CAR result in INFγ levels that are indistinguishable form the levels of INFγ release by a non-CAR containing cell under the same culture conditions. In other aspects, the tonic signaling is reduced by at least two-fold compared to a non-mutated scFv that is part of a CAR. In another aspect, the tonic signaling is reduced by at least five-fold compared to a non-mutated scFv that is part of a CAR. In another aspect, the tonic signaling is reduced by at least ten fold compared to a non-mutated scFv that is part of a CAR. In another aspect, the tonic signaling is reduced by at least fifty-fold compared to a non-mutated scFv that is part of a CAR. In aspects, tonic signaling is reduced by 100 fold, compared to a non-mutated scFv that is part of a CAR. In an aspect, tonic signaling is reduced between 10 and 100 fold, compared to a non-mutated scFv that is part of a CAR. In an aspect, tonic signaling is reduced between 5 and 50 fold, compared to a non-mutated scFv that is part of a CAR. In an aspect, tonic signaling is reduced between 10 and 200 fold, compared to a non-mutated scFv that is part of a CAR. In other aspects, the tonic signaling, as measured by the spontaneous release of INFγ, is reduced wherein the level of INFγ is less than 200 pg/ml/5×10⁵ cells. In other aspects, the tonic signaling, as measured by the spontaneous release of INFγ, is reduced wherein the level of INFγ is less than 100 pg/ml/5×10⁵ cells. In other aspects, the tonic signaling, as measured by the spontaneous release of INFγ, is reduced wherein the level of INFγ is less than 10 pg/ml/5×10⁵ cells. In aspects, the level of tonic signaling, after mutation of the scFv as part of a CAR according to the present disclosure is at or near the level of detection of INFγ. In aspects, immune cells having reduced tonic signaling have persistent expansion potential and increased functionality. In aspects, immune cells having reduced tonic signaling have persistent expansion potential, reduced immune cell exhaustion, and improved antitumor effects in vivo.

As provided by the present specification the method for reducing tonic signaling an mouse models comprise, the mouse immune cell is a natural killer T (NKT) cell, a T-cell, or a natural killer (NK) cell. In an aspect, the mouse immune cell is a T-cell. In another aspect, the mouse immune cell is an NKT cell. In a further aspect, the NKT cell is a Type-I NKT cell. In yet a further aspect, the NKT cell is a CD62L-positive Type-I NKT cell.

Unless defined otherwise, all technical and scientific terms used herein have the meaning commonly understood by a person skilled in the art to which this invent ion belongs. The following references provide one of skill with a general definition of many of the terms used in this invention: Singleton et al., Dictionary of Microbiology and Molecular Biology (2nd ed. 1994); The Cam bridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The terms “comprises”, “comprising”, “includes”, “including”, “having” and their conjugates mean “including but not limited to.” The term “consisting of” means “including and limited to.” The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a cell” or “at least one cell” may include a plurality of cells, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will encode a polypeptide that is identical to the recited sequences.

The terms “subject,” “individual,” and “patient,” are used interchangeably herein and refer to any vertebrate subject, including, without limitation, mammals, preferably a humans and other primates, including non-human primates such as laboratory animals including rodents such as mice, rats and guinea pigs; The term does not denote a particular age. Thus, both adult and newborn individuals are intended to be covered.

By “effective amount” is meant an amount sufficient to have a therapeutic effect. In one embodiment, an “effective amount” is an amount sufficient to arrest, ameliorate, or inhibit the continued proliferation, growth, or metastasis (e.g., invasion, or migration) of a neoplasia.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term, “operably linked”, as used herein, is meant the linking of two or more biomolecules so that the biological functions, activities, and/or structures associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker. In reference to nucleic acids, the term means that a first polynucleotide is positioned adjacent to a second polynucleotide that directs transcription of the first polynucleotide when appropriate molecules (e.g., transcriptional activator proteins) are bound to the second polynucleotide.

As used herein, “populations of cells” refer to pluralities of cells and may further comprise mixtures of different cell types as well as homogenous populations.

The term, “recognize” is meant selectively binds a target. An immune cell that recognizes a cell typically expresses a receptor that binds an antigen expressed by the cell. Immune cells in aspects according to the present disclosure express a CAR the binds to chondroitin sulfate proteoglycan 4 (CSPG4).

As used herein, an “autonomous intra-ribosomal self-processing peptide” is a small peptide of 18 amino acids that avoids the need of proteinases to process a polyprotein into separate proteins. First discovered in foot-and-mouth disease virus, when introduced as a linker between two proteins, these peptides provides for the autonomous intra-ribosomal self-processing of polyproteins. Similar sequences have been identified in other members of the pircornaviradae. See de Felipe, “Skipping the co-expression problem: the new 2A ‘CHYSEL’ technology,” Genetic Vaccines and Therapy 2:13 (2004).

As used herein, the term “engineering” refers to the genetic modification of a cell to introduce one or more exogenous nucleic acid sequences. Preferably, engineering introduced exogenous nucleic acid sequences that are transcribed and translated to express a protein. Introducing exogenous nucleic acid sequences can be performed using methods known in the art including transformation, transfection and transduction.

As used herein, the term “transcriptional activator in the Wnt signaling pathway” generally refers to proteins, that when exogenously expressed in a cell, activate genes downstream of Wnt/β-catenin signaling pathway. A transcriptional activator in the Wnt signaling pathway includes the expression of positive regulators of Wnt signaling such as LEF1 and inhibition of negative regulators, such as GSK3β. Also included in transcriptional activators of Wnt signaling are small molecule activators including, but not limited to those described in Blagodatski et al., “Small Molecule Wnt Pathway Modulators from Natural Sources: History, State of the Art and Perspectives,” Cells 9:589 (2020) and Verkaar et al., “Discovery of Novel Small Molecule Activators of β-catenin Signaling,” PLoS ONE 6(4): e19185 (2011) and include inhibitors of negative regulators of Wnt signaling, such as TWS119 (See Ding et al., Synthetic small molecules that control stem cell fate,” PNAS 100(13):7632-7 (2003)).

As used herein, the phrase “expresses a growth factor” refers to the exogenous expression of one or more growth factors, generally under the control of a heterologous promoter and more usually as part of a polyprotein downstream of a CHYSEL sequence.

While the present disclosure has been described with reference to certain embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

Any references cited herein are incorporated by reference in their entireties.

### EMBODIMENTS

Embodiment 1: A binding member having a binding specificity to chondroitin sulfate proteoglycan 4 (CSPG4), said binding member comprises a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382.

Embodiment 2: The binding member having a binding specificity to CSPG4 of embodiment 1, wherein said light chain framework sequence 1 (VL-FR1) is selected from the group consisting of SEQ ID NOs:153 to 158; said light chain framework sequence 2 (VL-FR2) is selected from the group consisting of SEQ ID NOs:222 to 225; said light chain framework sequence 3 (VL-FR3) is selected from the group consisting of SEQ ID NOs:226 to 231; and said light chain framework sequence 4 (VL-FR4) is selected from the group consisting of SEQ ID NOs:250 to 255; said heavy chain framework sequence 1 (VH-FR1) is selected from the group consisting of SEQ ID NOs:347 to 349; said heavy chain framework sequence 2 (VH-FR2) is selected from the group consisting of SEQ ID NOs:350 to 353; said heavy chain framework sequence 3 (VH-FR3) is selected from the group consisting of SEQ ID NOs:354 to 360; and said heavy chain framework sequence 4 (VH-FR4) is selected from the group consisting of SEQ ID NOs:361 to 362.

Embodiment 3: The binding member having a binding specificity to CSPG4 of embodiment 1 or 2, wherein said variable light chain sequence comprises a sequence selected from the group consisting of SEQ ID NOs:67 to 109 and said variable heavy chain sequence comprises a sequence selected from the group consisting of SEQ ID NOs:110 to 152.

Embodiment 4: A nucleic acid encoding a polypeptide comprising a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255.

Embodiment 5: A nucleic acid encoding a polypeptide comprising a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382.

Embodiment 6: A chimeric antigen receptor (CAR) expression construct comprising nucleic acid sequences encoding a chimeric antigen receptor (CAR) coding sequence comprising an ectodomain sequence comprising an antibody or antigen binding fragment thereof that binds to chondroitin sulfate proteoglycan 4 (CSPG4) comprising variable light chain sequence comprising light chain complementarity determining regions (“LCDRs”) LCDR1 to LCDR3 sequences set forth in SEQ ID NOs:1-3 a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence.

Embodiment 7: The chimeric antigen receptor (CAR) expression construct of embodiment 6, wherein said transmembrane domain is selected from the group consisting of CD28 (Gene ID:940, 12487), CD3-ζ (Gene ID:919; 12503 CD247), CD4 (Gene ID:920, 12504), CD8 (Gene ID:924, 12525), CD16 (Gene ID:2214; 14131; Fcgr3), NKp44 (Gene ID:9436, NCR2), NKp46 (Gene ID:9437, 17086, NCR1), and NKG2d (Gene ID:22914; 27007 KLRK1).

Embodiment 8: The chimeric antigen receptor (CAR) expression construct of embodiment 6 or 7, wherein said endodomain sequence is selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

Embodiment 9: The chimeric antigen receptor (CAR) expression construct of any one of embodiments 6 to 8, further comprising sequences encoding a protein sequence for a transcriptional activator in the Wnt signaling pathway.

Embodiment 10: The chimeric antigen receptor (CAR) expression construct of any one of embodiments 6 to 9, wherein said expression construct encodes a polyprotein comprising said protein sequence for a transcriptional activator in the Wnt signaling pathway and up to three additional protein coding sequences.

Embodiment 11: The chimeric antigen receptor (CAR) expression construct of any one of embodiments 6 to 10, wherein said protein sequence for a transcriptional activator in the Wnt signaling pathway and up to three additional protein coding sequences are separated by an autonomous intra-ribosomal self-processing peptide.

Embodiment 12: The chimeric antigen receptor (CAR) expression construct of any one of embodiments 6 to 11, wherein said autonomous intra-ribosomal self-processing is a foot-and-mouth disease virus (FMDV) 2A sequence or a related cis acting hydrolase element (CHYSEL).

Embodiment 13: The chimeric antigen receptor (CAR) expression construct of any one of embodiments 6 to 12, wherein said transcriptional activator is selected from the group consisting of lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) and TLE family member 1, transcriptional corepressor (TLE 1, Gene ID 7088).

Embodiment 14: The chimeric antigen receptor (CAR) expression construct of any one of embodiments 6 to 13, wherein said LEF1 selected from the group consisting of Reference Sequence (RefSeq) ID NOs: NP_057353.1, NP_001124185.1, and NP_001124186.1.

Embodiment 15: The chimeric antigen receptor (CAR) expression construct of any one of embodiments 6 to 14, further comprising at least one protein coding sequence for a growth factor.

Embodiment 16: The chimeric antigen receptor (CAR) expression construct of any one of embodiments 6 to 15, wherein said growth factor is selected from the group consisting of interleukin-15 (IL-15), interleukin-7 (IL-7), interleukin-12 (IL-12), interleukin-18 (IL-18), interleukin-21 (IL-21), interleukin-27 (IL-27), interleukin-33 (IL-33), and combinations thereof.

Embodiment 17: The chimeric antigen receptor (CAR) expression construct of any one of embodiments 6 to 16, wherein said protein coding sequence for a growth factor is separated from said CAR coding sequence by a foot-and-mouth disease virus (FMDV) 2A sequence or a FMDV 2A related cis acting hydrolase element (CHYSEL) sequence.

Embodiment 18: The chimeric antigen receptor (CAR) expression construct of any one of embodiments 6 to 17, wherein said ectodomain sequences further comprises a spacer domain.

Embodiment 19: The chimeric antigen receptor (CAR) expression construct of any one of embodiments 6 to 18, wherein said endodomain comprises the signal sequence of 4-1BB fused in-frame to a CD3-zeta chain.

Embodiment 20: The chimeric antigen receptor (CAR) expression construct of any one of embodiments 6 to 19, further comprising a DNA sequence encoding a small hairpin RNA (shRNA) sequence targeting an MHC class I or MHC class II gene, wherein the shRNA sequence is embedded in an artificial microRNA (amiR) scaffold.

Embodiment 21: A chimeric antigen receptor (CAR) comprising: an ectodomain sequence comprising an antibody or antigen binding fragment thereof that binds to chondroitin sulfate proteoglycan 4 (CSPG4) selected from the group consisting of SEQ ID NOs:28 to 65; a transmembrane domain sequence; and an endodomain sequence.

Embodiment 22: The chimeric antigen receptor (CAR) of embodiment 21, wherein said ectodomain sequence is selected from the group consisting of SEQ ID NOs:34, 46, 47, and 57.

Embodiment 23: The chimeric antigen receptor (CAR) of embodiment 21 or 22, wherein said ectodomain sequence comprises SEQ ID NO:34.

Embodiment 24: The chimeric antigen receptor (CAR) of embodiment 2 or 221, wherein said ectodomain sequence comprises SEQ ID NO:46.

Embodiment 25: The chimeric antigen receptor (CAR) of embodiment 21 or 22, wherein said ectodomain sequence comprises SEQ ID NO:47.

Embodiment 26: The chimeric antigen receptor (CAR) of embodiment 21 or 22, wherein said ectodomain sequence comprises SEQ ID NO:57.

Embodiment 27: The chimeric antigen receptor (CAR) of any one of embodiments 21 to 26, wherein said a transmembrane domain sequence selected from the group consisting of CD28 (Gene ID:940, 12487), CD3-ζ (Gene ID:919; 12503 CD247), CD4 (Gene ID:920, 12504), CD8 (Gene ID:924, 12525), CD16 (Gene ID:2214; 14131; Fcgr3), NKp44 (Gene ID:9436, NCR2), NKp46 (Gene ID:9437, 17086, NCR1), and NKG2d (Gene ID:22914; 27007 KLRK1)

Embodiment 28: The chimeric antigen receptor (CAR) of any one of embodiments 21 to 27, wherein said endodomain sequence is selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

Embodiment 29: A nucleic acid molecule encoding a CAR comprising an ectodomain sequence comprising an antibody or antigen binding fragment thereof that binds to chondroitin sulfate proteoglycan 4 (CSPG4) selected from the group consisting of SEQ ID NOs:28 to 65; a transmembrane domain sequence; and an endodomain sequence.

Embodiment 30: The nucleic acid molecule encoding a CAR of embodiment 29, wherein said ectodomain sequence is selected from the group consisting of SEQ ID NOs:34, 46, 47, and 57.

Embodiment 31: The nucleic acid molecule encoding a CAR of embodiment 29 or 30, wherein said ectodomain sequence comprises SEQ ID NO:34.

Embodiment 32: The nucleic acid molecule encoding a CAR of embodiment 29 or 30, wherein said ectodomain sequence comprises SEQ ID NO:46.

Embodiment 33: The nucleic acid molecule encoding a CAR of embodiment 29 or 30, wherein said ectodomain sequence comprises SEQ ID NO:47.

Embodiment 34: The nucleic acid molecule encoding a CAR of embodiment 29 or 30, wherein said ectodomain sequence comprises SEQ ID NO:57.

Embodiment 35: The nucleic acid molecule encoding a CAR of any one of embodiments 29 to 34, wherein said a transmembrane domain sequence selected from the group consisting of CD28 (Gene ID:940, 12487), CD3-ζ (Gene ID:919; 12503 CD247), CD4 (Gene ID:920, 12504), CD8 (Gene ID:924, 12525), CD16 (Gene ID:2214; 14131; Fcgr3), NKp44 (Gene ID:9436, NCR2), NKp46 (Gene ID:9437, 17086, NCR1), and NKG2d (Gene ID:22914; 27007 KLRK1)

Embodiment 36: The nucleic acid molecule encoding a CAR of any one of embodiments 29 to 35, wherein said endodomain sequence is selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

Embodiment 37: A host cell transformed or transfected with the expression construct as defined in any one of embodiments 6 to 20 or with the nucleic acid sequence as defined in any one of embodiments 29 to 35.

Embodiment 38: The host cell of embodiment 37, wherein said host cell is selected from the group consisting of a bacteria, a natural killer T (NKT) cell, a T-cell, and a natural killer (NK) cell.

Embodiment 39: The host cell of embodiment 37 or 38, wherein said host cell is a T-cell.

Embodiment 40: The host cell of embodiment 37 or 38, wherein said host cell is an NKT cell.

Embodiment 41: The host cell of any one of embodiments 37, 38, or 40, wherein said host cell is a Type-I NKT cell.

Embodiment 42: A process for the production of an immune cell comprising a CAR isolating PBMCs from a donor; separating a natural killer T (NKT) cells, T-cells, or natural killer (NK) cells from said PBMCs to prepare isolated immune cells; and expanding said isolated immune cells for between 1 and 20 days to prepare expanded immune cells for genetic engineering by stimulation of an endogenous T-cell receptor and co-stimulation by costimulatory receptors, cytokines, or a combination of both; and introducing a chimeric antigen receptor (CAR) expression construct comprising an ectodomain sequence comprising an antibody or antigen binding fragment thereof that binds to chondroitin sulfate proteoglycan 4 (CSPG4) comprising a variable light chain selected from the group consisting of SEQ ID NOs:61 to 109 and a variable heavy chain selected from the group consisting of SEQ ID NOs:110 to 152.

Embodiment 43: The process for the production of an immune cell comprising a CAR of embodiment 42, wherein said isolated immune cells are T cells.

Embodiment 44: The process for the production of an immune cell comprising a CAR of embodiment 41 or 42, wherein said isolated immune cells are NK cells.

Embodiment 45: The process for the production of an immune cell comprising a CAR of embodiment 41 or 42, wherein said isolated immune cells are Type I NKT cells.

Embodiment 46: The process for the production of an immune cell comprising a CAR of any one of embodiments 41, 42, or 43, wherein said expanded immune cells are CD62L positive Type I NKT cells.

Embodiment 47: The process for the production of an immune cell comprising a CAR of embodiment 45 or 46 wherein said isolated immune cells are Type I NKT cells and said expanding comprises culturing in the presence of at least aGalCer, IL-2, and IL-21.

Embodiment 48: A genetically engineered immune cell comprising an expression construct encoding a chimeric antigen receptor (CAR) coding sequence comprising an ectodomain sequence comprising an antibody or antigen binding fragment thereof that binds to chondroitin sulfate proteoglycan 4 (CSPG4) variable light chain sequence comprising light chain complementarity determining regions (“LCDRs”) LCDR1 to LCDR3 sequences set forth in SEQ ID NOs:1-3 a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence.

Embodiment 49: The genetically engineered immune cell of embodiment 47 or 48, wherein said genetically engineered immune cell is a natural killer T (NKT) cell, a T-cell, or a natural killer (NK) cell.

Embodiment 50: The genetically engineered immune cell of embodiment 47 or 49, wherein said host cell is a T-cell.

Embodiment 51: The genetically engineered immune cell of embodiment 47 or 49, wherein said host cell is an NKT cell.

Embodiment 52: The genetically engineered immune cell of any one of embodiments 47, 49, or 51, wherein said host cell is a Type-I NKT cell.

Embodiment 53: The genetically engineered immune cell of any one of embodiments 47, 49, 51, or 52, wherein said host cell is a CD62L-positive Type-I NKT cell.

Embodiment 54: The genetically engineered immune cell of embodiment 48, wherein said genetically engineered immune cell comprises a plurality of cells.

Embodiment 55: The genetically engineered immune cell of embodiment 48 or 54, wherein said genetically engineered immune cell comprises a plurality of CD62L-positive Type I NKT cells.

Embodiment 56: The genetically engineered immune cell of any one of embodiments 48, 54, or 55, wherein said plurality of CD62L-positive Type I NKT cells comprise at least 50% of said plurality of cells.

Embodiment 57: The genetically engineered immune cell of any one of embodiments 48 to 56, further comprising a DNA sequence encoding a small hairpin RNA (shRNA) sequence targeting an MHC class I or MHC class II gene, wherein the shRNA sequence is embedded in an artificial microRNA (amiR) scaffold.

Embodiment 58: A population of cells comprising a plurality of genetically engineered immune cells comprising an expression construct encoding a chimeric antigen receptor (CAR) coding sequence comprising an ectodomain sequence comprising an antibody or antigen binding fragment thereof that binds to chondroitin sulfate proteoglycan 4 (CSPG4), said CAR comprising a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence.

Embodiment 59: The population of cells of embodiment 58, wherein said genetically engineered immune cells comprise a natural killer T (NKT) cells, T-cells, or natural killer (NK) cells.

Embodiment 60: The population of cells of embodiment 58 or 59, wherein said genetically engineered immune cell comprises a plurality of CD62L-positive Type I NKT cells.

Embodiment 61: The population of cells of embodiment 58, 59 or 60, wherein said plurality of CD62L-positive Type I NKT cells comprise at least 50% of said plurality of cells.

Embodiment 62: A method of inhibiting chondroitin sulfate proteoglycan 4 (CSPG4)-positive cells in an individual, comprising the step of contacting the cells with a therapeutically effective amount of genetically engineered immune cells, wherein said immune cells comprise a chimeric antigen receptor (CAR) comprising an ectodomain sequence comprising an antibody or antigen binding fragment thereof that binds to chondroitin sulfate proteoglycan 4 (CSPG4), said antibody or antigen binding fragment comprising: a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“LCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence.

Embodiment 63: The method according to embodiment 62, wherein said inhibiting comprises inhibiting proliferation, inhibiting activity, or a combination of both.

Embodiment 64: The method according to embodiment 62 or 63, wherein said genetically engineered immune cells are natural killer T (NKT) cells, T-cells, or natural killer (NK) cells.

Embodiment 65: The method according to any one of embodiments 62 to 64, wherein said genetically engineered immune cells are T-cells.

Embodiment 66: The method according to any one of embodiments 62 to 64, wherein said genetically engineered immune cells are NKT cells.

Embodiment 67: The method according to any one of embodiments 62 to 64 or 66, wherein said NKT cells are Type-I NKT cells.

Embodiment 68: The method according to any one of embodiments 62 to 64, 66 or 67, wherein said Type-I NKT cells are CD62L-positive Type-I NKT cells.

Embodiment 69: The method according to any one of embodiments 62 to 64, or 66 to 68, wherein said Type-I NKT cells comprise a majority of said genetically engineered immune cells.

Embodiment 70: The method according to any one of embodiments 62 to 64, or 66 to 69, wherein said Type-I NKT cells comprise a majority of said genetically engineered CD62L-positive Type-I NKT cells.

Embodiment 71: A method for the treatment of cancer, comprising the step of administering to a subject in need thereof the genetically engineered immune cells comprising a chimeric antigen receptor (CAR) that binds to chondroitin sulfate proteoglycan 4 (CSPG4), said CAR comprising an ectodomain sequence comprising: a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence.

Embodiment 72: The method according to embodiment 71, wherein said cancer is selected from the group consisting of melanoma, metastatic melanoma disease [superficial spreading melanoma, lentigo maligna, lentigo maligna melanoma, acral lentiginous melanoma and nodular melanoma]; glioblastoma, anaplastic thyroid cancer, soft tissue sarcoma, glioma and leukemia

Embodiment 73: The method for the treatment of cancer of embodiment 71 or 72, wherein said genetically engineered immune cells are natural killer T (NKT) cells, T-cells, or natural killer (NK) cells.

Embodiment 74: The method for the treatment of cancer of any one of embodiments 71 to 73, wherein said genetically engineered immune cells are T-cells.

Embodiment 75: The method for the treatment of cancer of any one of embodiments 71, 72 or 73, wherein said genetically engineered immune cells are NKT cells.

Embodiment 76: The method for the treatment of cancer of any one of embodiments 71, 72 or 75, wherein said NKT cells are Type-I NKT cells.

Embodiment 77: The method for the treatment of cancer of any one of embodiments 71, 72, 75, or 76, wherein said Type-I NKT cells are CD62L-positive Type-I NKT cells.

Embodiment 78: The method for the treatment of cancer of any one of embodiments 71, 72, or 75 to 77, wherein said Type-I NKT cells comprise a majority of said genetically engineered immune cells.

Embodiment 79: The method for the treatment of cancer of any one of embodiments 71, 72, or 75 to 78, wherein said Type-I NKT cells comprise a majority of said genetically engineered CD62L-positive Type-I NKT cells.

Embodiment 80: A kit comprising an a vector, a host cell, or a combination thereof comprising nucleic acid sequences encoding a chimeric antigen receptor (CAR) coding sequence comprising an ectodomain sequence comprising an antibody or antigen binding fragment thereof that binds to chondroitin sulfate proteoglycan 4 (CSPG4) comprising a variable light chain sequence comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs: 4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence.

Embodiment 81: A method of maintaining NKT cell expansion potential in NKT cells expressing a chimeric antigen receptor (CAR) coding sequence comprising an ectodomain sequence comprising an antibody or antigen binding fragment thereof that binds to chondroitin sulfate proteoglycan 4 (CSPG4) comprising light chain complementarity determining regions (“LCDRs”) LCDR1 to LCDR3 sequences set forth in SEQ ID NOs:1-3 and a light chain framework sequence 1 (VL-FR1) selected from the group consisting of SEQ ID NOs:153 to 221; a light chain framework sequence 2 (VL-FR2) selected from the group consisting of SEQ ID NOs:222 to 225; a light chain framework sequence 3 (VL-FR3) selected from the group consisting of SEQ ID NOs:226 to 249; and a light chain framework sequence 4 (VL-FR4) selected from the group consisting of SEQ ID NOs:250 to 255; a variable heavy chain sequence comprising heavy chain complementarity determining regions (“HCDRs”) HCDR1 to HCDR3 sequences set forth in SEQ ID NOs:4 to 6 and a heavy chain framework sequence 1 (VH-FR1) selected from the group consisting of SEQ ID NOs:256 to 349; a heavy chain framework sequence 2 (VH-FR2) selected from the group consisting of SEQ ID NOs:350 to 353; a heavy chain framework sequence 3 (VH-FR3) selected from the group consisting of SEQ ID NOs:354 to 360; and a heavy chain framework sequence 4 (VH-FR4) selected from the group consisting of SEQ ID NOs:361 to 382; a transmembrane domain sequence; and an endodomain sequence, said method comprising expressing a protein coding sequence comprising a transcriptional activator in the Wnt signaling pathway, and culturing said engineered NKT cells to prepare a population of genetically engineered NKT cells with persistent expansion potential.

Embodiment 82: A method of reducing tonic signaling in mouse models in an scFv comprising: identifying an scFv that has tonic signaling when expressed in a mouse immune cell as part of a CAR; generating a structural model of said scFv, and performing computational mutagenesis to prepare a series of mutagenized scFvs; calculating the free energy of said mutagenized scFvs; aligning said mutagenized scFvs to a humanized scFv comprising framework 1.4 (FW1.4) identifying critical murine residues; and introducing one or more human to mouse residue changes to increase stability of said scFv and prepare a modified humanized scFv for use in mouse models.

Embodiment 83: A method of reducing tonic signaling in mouse models in an scFv of embodiment 82, wherein FW1.4 comprises the light chain framework regions (VL-FR) 1 to 4 of SEQ ID NOs:7 to 10, a linker region of SEQ ID NO:11, and heavy chain framework region (VH-FR) 1 to 4 of SEQ ID NOs:12 to 15.

Embodiment 84: A method of reducing tonic signaling in mouse models in an scFv of embodiment 82 or 83, wherein said modified humanized scFv comprises modified a modified FR region selected from the group consisting of SEQ ID NOs:16 to 27.

Embodiment 85: A method of reducing tonic signaling in mouse models in an scFv of any one of embodiments 82 to 84, wherein said immune cell is a natural killer T (NKT) cell, a T-cell, or a natural killer (NK) cell.

Embodiment 86: A method of reducing tonic signaling in mouse models in an scFv of any one of embodiments 82 to 85, wherein said immune cell is T-cell.

Embodiment 87: A method of reducing tonic signaling in mouse models in an scFv of any one of embodiments 82 to 85, wherein said immune cell is an NKT cell.

Embodiment 88: A method of reducing tonic signaling in mouse models in an scFv of any one of embodiments 82 to 85, or 87, wherein said NKT cell is a Type-I NKT cell.

Embodiment 89: A method of reducing tonic signaling in mouse models in an scFv of any one of embodiments 82 to 85, 87 or 88, wherein said Type-I NKT cell is a CD62L-positive Type-I NKT cell.

### EXAMPLES

Cell lines. The tumor cell lines WM115 (melanoma) and SK-MEL-2 are obtained from the American Type Culture Collection (ATCC) (CRL-1675). M14 cells was provided by Dr Ferrone. The tumor cell lines MDA-MB-468 and MDA-MB-231 are obtained from German Collection of Microorganism and Cell Cultures GmbH (ACC 738 and ACC 732). 293T cells used for the production of retroviral vectors are obtained from the ATCC. All cells are maintained in culture with the appropriate media, either RPMI-1640 (Gibco) or DMEM (Gibco) supplemented with 10% FBS (Sigma), 1% L-glutamine (Gibco), and 1% penicillin/streptomycin (Gibco) in a humidified atmosphere containing 5% CO2 at 37° C. WM115 cells are transduced with an SFG gamma retroviral vector encoding the firefly luciferase gene and the fusion protein enhanced GFP (eGFP-FFluc). See Hoyos et al., “Engineering CD19-specific T lymphocytes with interleukin-15 and a suicide gene to enhance their anti-lymphoma/leukemia effects and safety,” Leukemia 24:1160-1170 (2010). Cells are kept in culture for less than 6 consecutive months, after which aliquots from the original expanded vial were used. All tumor cell lines are routinely tested to exclude contamination with Mycoplasma and assessed for the expression of transgenes and tumor markers by flow cytometry to confirm identity. Glioblastoma-derived neurospheres are generated as previously described. See Pellegatta et al., “Constitutive and TNFalpha-inducible expression of chondroitin sulfate proteoglycan 4 in glioblastoma and neurospheres: Implications for CAR-T cell therapy,” Sci. Transl. Med. 10 (2018) (“Pellegatta et al., (2018)”).

Generation of retroviral supernatants, T-cell isolation, transduction, and in vitro expansion. Retroviral supernatants are prepared by transient transfection of 293T cells and used to transduce T cells. See Diaconu et al., “Inducible Caspase-9 Selectively Modulates the Toxicities of CD19-Specific Chimeric Antigen Receptor-Modified T Cells,” Mol. Ther. 25:580-592 (2017) (“Diaconu et al. 2017”). Buffy coats from healthy volunteer blood donors are purchased from the Gulf Coast Regional Blood Center (Houston, Tex.). Peripheral blood mononuclear cells (PBMC) are isolated by Lymphoprep (Accurate Chemical and Scientific Corporation) density gradient centrifugation, according to the manufacturer's protocol. T cells are cultured in complete medium, consisting of 45% Click's medium (Irvine Scientific), 45% RPMI-1640 (Hyclone), 10% FBS (Hyclone), 1% L-glutamine (Gibco), and 1% penicillin/streptomycin (Gibco). T cells are activated, transduced, and expanded in complete medium with IL7 (10 ng/mL, PeproTech) and IL15 (5 ng/mL, PeproTech) as previously reported. See Diaconu et al. 2017.

Immunophenotyping. T cells are stained with antibodies (Ab) against CD3 (APC-H7, clone SK7), CD45Ra (PE, clone HI100), CCR7 (FITC, clone 150503), CTLA4 (BV421, clone BNI3), PD-1 (PE-Cy7, clone EH12.1), LAG3 (PE, clone T47-530), TIM3 (BV711, clone 7D3) an CD45 (APC, clone 2D1) from BD Biosciences. Anti-CD45 (PerCP, clone REA747) and anti CD69 (APC, clone REA824) from REAffinity by Miltenyi Biotec. Tumor cells are stained with Abs against CD276 (BV421, clone 7-517) from BD Biosciences and with the 763.74 mAb (anti-CSPG4) followed by the staining with a secondary Rat anti-Mouse IgG₁ (PE, clone X56) from BD Biosciences. The expression of the 763.74(A) and (B) CAR is assessed using an anti-idyotipic antibody, the expression of CTR CAR (anti-CD19 CAR) is assessed using an anti-idiotypic antibody (obtained from Dr Ferrone), followed by the staining with a secondary Rat anti-Mouse IgG₁ (PE, clone X56) from BD Biosciences. The expression of the h763.74 CAR followed by the staining with Streptavidin Protein RPE conjugate from Invitrogen. Data acquisition is performed on BD LSRFortessa or Canto II flow cytometer using the BD FACS-Diva software or on a MACSQuant (Miltenyi Biotec). Data analyses are performed with the FlowJo software (Version 9 or 10) or FlowLogic software (Version 7.2, Miltenyi Biotec).

Confocal microscopy. T cells are prepared according to the manufacturer's instructions (Abcam immunofluorescence protocol). Briefly, CAR-GFP⁺ cells are fixed with cytofix buffer (BD Biosciences) and are mounted on cover slips with one drop of ProLong Diamond Antifade Mountant with DAPI (Invitrogen). Data acquisition is performed on LSM700 Zeiss confocal microscopy using ZEN software (ZEISS Microscopy). Data analysis is performed with Fiji software.

Coculture experiments and ELISAs. For the spontaneous IFNγ release assay, 1×10⁶ T cells are plated in 24 well plate in 2 mL of complete media without cytokines. T cells (2×10⁴ cells/well) are cocultured with tumor cell lines (M14-wt or WM115; 10⁵ cells/well) at an effector-to-target (E:T) ratio of 1:5 in 24-well plates, in complete medium, in the absence of cytokines. After 5 days of culture, cells are harvested and stained for CD3 (APC-H7, clone SK7 from BD Biosciences) and CD276 (BV421, clone 7-517 from BD Biosciences) monoclonal Abs (mAb) to detect T cells and tumor cells, respectively. See Landoni et al., “A High-Avidity T-cell Receptor Redirects Natural Killer T-cell Specificity and Outcompetes the Endogenous Invariant T-cell Receptor,” Cancer Immunol. Res. 8:57-69 (2020). Percentage of residual tumor cells in culture are enumerated by flow cytometry. Spontaneous release and culture supernatants are harvested after 24 hours of culture and IFNγ measured in 100 mL of supernatant with the DuoSet Human IFNγ ELISA kit (R&D Systems). Data acquisition is performed on a Synergy2 microplate reader (BioTek) using the Gen5 software. GBM-NS are plated at 5×10⁵ cells in 24-well plates with T cells at E:T ratio of 1:5 in GBM-NS medium without serum and in the presence of B27 supplement. T cells are maintained in GBM-NS medium for 3 days before plating the co-cultures. See Pellegatta et al. (2018). GBM-NS and T cells are collected at different time points following 2, 4, 6 and 24 hours of co-culture, and residual tumor cells and T cells measured by flow cytometry based on CSPG4 and CD45 expression, respectively. The activation of CAR-T cells is measured by evaluating the expression of CD69.

Computational analysis. To generate the 3D conformation of scFv, the primary sequence of scFv is BLAST searched against the RCSB database to identify homologous template structures with high sequence similarity. BLASTp analysis identifies scFv fragment 1696 with resolution 2.70 Å as a potential template with 70.51% sequence identity. See Rezacova et al., “Structural basis of HIV-1 and HIV-2 protease inhibition by a monoclonal antibody,” Structure. 9:887-895 (2001) (“Rezacova et al., (2001)”). The crystal structure of scFv fragment 1696 (PDB ID: 1jp5) is used as template to model scFv through homology modeling. Id. Forty models using Modeller-9v19 are generated and the structure selected with least molecular objective function score as the representative conformation of scFv. See Webb and Sali, “Comparative Protein Structure Modeling Using MODELLER,” Curr. Protoc. Protein Sci. 86:2 (2016). Since steric clashes are common in modelled and low-resolution structures, Chiron is employed to optimize the structure of scFv. See Kota et al., “Gaia: automated quality assessment of protein structure models,” Bioinformatics. 27:2209-2215 (2011). Chiron resolves atomic clashes by performing short-DMD simulations on protein structure with minimal or no perturbation to the backbone. See Ding et al., “Ab initio folding of proteins with all-atom discrete molecular dynamics,” Structure. 16:1010-1018 (2008); Shirvanyants et al., “Discrete molecular dynamics: an efficient and versatile simulation method for fine protein characterization,” J Phys. Chem. B 116:8375-8382 (2012); and Dokholyan et al., “Discrete molecular dynamics studies of the folding of a protein-like model,” Fold. Des 3:577-587 (1998). The relaxed scFv1 structure is subsequently considered for in silico mutagenesis studies using Eris molecular suite. See Yin et al., “Eris: an automated estimator of protein stability,” Nat. Methods 4:466-467 (2007). Eris protocol induces mutations in protein and estimates free energies of mutant (ΔGₘᵤₜ) and wild type (ΔGₘᵤₜ) conformations. Eris performs rapid side-chain repacking and backbone relaxation around the mutated site using Monte-Carlo algorithm and subsequently evaluates ΔG_{wt} and ΔGₘᵤₜ using Medusa force field. See id.; and Yin et al., “MedusaScore: an accurate force field-based scoring function for virtual drug screening,” J Chem. Inf. Model. 48:1656-1662 (2008). The Eris algorithm computes change in free energy of protein upon mutation by employing the following formula: ΔΔGmut=ΔGₘᵤₜ−ΔGₘᵤₜ. The ΔΔGmut values are evaluated to estimate the stabilizing (ΔΔGₘᵤₜ<0) or destabilizing (ΔΔGₘᵤₜ>0) mutations. Eris is extensively validated and used in designing novel proteins. See Zhu et al., “Rationally designed carbohydrate-occluded epitopes elicit HIV-1 Env-specific antibodies,” Nat. Commun. 10:948 (2019); Dagliyan et al., “Engineering extrinsic disorder to control protein activity in living cells,” Science 354:1441-1444 (2016); and Dagliyan et al., “Rational design of a ligand-controlled protein conformational switch,” Proc. Natl. Acad. Sci. U.S.A 110:6800-6804 (2013).

Xenograft models. Melanoma mouse experiments are performed in accordance with UNC Animal Husbandry and Institutional Animal Care and Use Committee (IACUC) guidelines and were approved by UNC IACUC (ID: 17029). GBM mouse experiments are performed following directives of Fondazione IRCCS Istituto Neurologico Carlo Besta in Milan in accordance with the Italian Principle of Laboratory Animal Care (D. Lgs. 26/2014) and European Communities Council Directives (86/609/EEC and 2010/63/UE). For the melanoma model, female and male NSG mice (7-9 weeks of age, obtained from the UNC Animal Core) are injected subcutaneously (s.c.) with 0.5×10⁶ eGFP-FFluc-labeled WM115 tumor cells. Seven days after tumor cell injection (day 0) mice are infused i.v. with 5×10⁶ CAR-T cells. Melanoma tumor cell growth is monitored weekly with caliper measurement for s.c. tumors and by bioluminescence (BLI; total flux, photons/second) using the IVIS kinetic in vivo imaging system (PerkinElmer). Mice are sacrificed according to UNC guidelines for tumor growth or occurrence of sign of discomfort. When mice are sacrificed, peripheral blood is collected from heart, spleen and liver smashed on cell strainers and washed with 2 mL of PBS. Peripheral blood, spleen and liver are analyzed to detect the presence of T cells [stained with Abs against CD3 (APC-H7, clone SK7), CD45 (APC, clone 2D1), PD-1 (PE/Cy7, clone EH12.1) and CAR-specific anti-idiotype] by flow cytometry using CountBright absolute counting beads (Invitrogen). For the GBM model, antitumor activity of CAR-T cells is evaluated using nude mice engrafted with GBM-NS. Five to 6-week-old mice are injected intra caudate nucleus (i.c.) with 0.1×10⁶ GBM-NS in 2 μL PBS 1×. The coordinates, with respect to the bregma, are 0.7 mm post, 3 mm left lateral, 3.5 mm deep, and within the nucleus caudatum. On day 15 after tumor cell injection, CAR-T cells are injected i.c. in 5 μL PBS 1× using the same tumor coordinates. For survival studies, mice are monitored three times a week and euthanized when signs of discomfort appeared in accordance with the institutional guidelines.

Magnetic Resonance Imaging (MM). MRI is performed using a horizontal-bore preclinical scanner (BioSpec 70/20 USR, Bruker, Ettlingen, Germany). The system has a magnetic field strength of 7 T (1H frequency 300 MHz) and a 20 cm bore diameter. The scanner is equipped with an actively shielded gradient system with integrated shims set up to 2nd order. The maximum gradient amplitude is 440 mT/m. All acquisitions are carried out using a cross coil configuration: a 72 mm linear birdcage coil is used for radiofrequency excitation and a mouse brain surface coil received signal. Mice are anaesthetized with 1.5-2% isoflurane (60:40 N20:02 (vol:vol), flow rate 0.8 L/min). To detect the depth of anesthesia and the animal health condition during the study, the respiratory rate is monitored by a pneumatic sensor. Mice are positioned on an animal bed equipped with a nose cone for gas anesthesia and a three point-fixation system (tooth-bar and ear-plugs). Mice injected with GBM-NS and treated with CAR-T cells undergo high resolution MM investigation at different time points with the following protocol: a T2-weighted Rapid Acquisition with Reduced Echoes (RARE) sequence (TR=3360 ms, TE=35 ms, in plane resolution=100×100 um², slice thickness=400 um, 4 averages, total acquisition time of 5 min 36 sec) and two T1-weighted RARE sequences (TR=510 ms, TE=8 ms, in plane resolution=78×78 um2, slice thickness=400 um, 6 averages, total acquisition time of 9 min 47 sec) acquired before and after intraperitoneal administration of Gadoliunium-based contrast medium. All sequences are acquired along the same coronal geometry (400 um thick continuous slices), with slice package posterior to olfactory bulb and anterior to cerebellum. Contrast agent induced T1 signal enhancement is interpreted as being due to a Blood Brain Barrier lesion.

Humanization of the scFv 763.74(A). Humanization of the murine scFv 763.74(A) is performed by grafting its CDRs into the stable human framework rFW1.4. See Borras et al., “Generic approach for the generation of stable humanized single-chain Fv fragments from rabbit monoclonal antibodies, J Biol. Chem. 285:9054-9066 (2010). The sequence of the FW, referred here as rFW1.4 is as follows:

LCDR1*
(SEQ ID NO: 7)
EIVMTQSPSTLSASVGDRVIITC *
LCDR2*
(SEQ ID NO: 8)
WYQQKPGKAPKLLIY*
LCDR3*
(SEQ ID NO: 9)
GVPSRFSGSGSGTEFTLTISSLQPDDFATYYC*
(SEQ ID NO: 10)
FGQGTKLTVLG
(SEQ ID NO: 11)
(GGGGSGGGGSGGGGSGGGGS)
HCDR1*
(SEQ ID NO: 12)
EVQLVESGGGLVQPGGSLRLSCTASG*
HCDR2*
(SEQ ID NO: 13)
WVRQAPGKGLEWVG*
HCDR3*
(SEQ ID NO: 14)
RFTISRDTSKNTVYLQMNSLRAEDTAVYYCAR*
(SEQ ID NO: 15)
WGQGTLVTVSS.

Asterisks separate the amino acid sequence of the framework from the CDR sequences. A polypeptide linker consisting of (Gly₄Ser)₄ is used to join the V_{L} and V_{H} chains and shown in round brackets. Humanized versions of 763.74(A) are designed by replacing human framework residues with the critical murine residues. See Yin, S., Ding, F., and Dokholyan, N. V. 2007. Eris: an automated estimator of protein stability. Nat. Methods 4:466-467.

Expression and Purification of scFv fragments. E. coli BL21(DE3) transformed with the corresponding expression plasmids are grown at 37° C. in LB medium containing the appropriate antibiotics. Protein expression is initiated by addition of 1 mM isopropyl 1-thio-β-d-galactopyranoside at an absorbance (A600) of 1. Four hours after induction, E. coli cells are harvested and disrupted by sonication. Inclusion bodies are isolated by repeated washing and centrifugation steps and solubilized at a concentration of 10 mg/mL in the presence of 6 M Guanidine HCl. Solubilized inclusion bodies are reduced by adding 20 mM dithiothreitol. Refolding is done in refolding buffer (4 M Urea, 50 mM Glycine, 2 mM Cystine, 2 mM Cysteine pH 10.0) over night at room temperature. After up-concentration and buffer exchange using tangential flow filtration with a 10 kDa cut-off scFvs are purified using hydrophobic interaction chromatography followed by size exclusion chromatography.

Binding studies of scFvs. Binding studies are performed using CSPG4⁺MDA-MB-231 and CSPG4⁻ MDA-MB-468 cells by flow cytometry. Cells are incubated with the respective scFv, biotinylated Protein L and finally with streptavidin-phycoerythrin. Cells are acquired on a FACSAria III (BD Biosciences) instrument using the FACSDiva software.

Stability of scFvs. Humanized scFvs are formulated in PBS pH-7.2 at 1 mg/ml. After 48 h of storage at 4° C. or 37° C., the samples are inspected visually, and protein concentration is measured at 280 nm. The samples are analyzed by SEC-HPLC to determine the percentage of monomers, dimers and high molecular weight oligomers in relation to total peak area. A TSKgel G2000 SWXL column, phase diol, L×I.D. 30 cm×7.8 mm, 5 μm particle size (Sigma-Aldrich, 08540) is used for size exclusion chromatography. Five (5) μL of scFvs at 1 mg/mL are loaded. The mobile phase is PBS pH 7.2.

Statistical analysis. Data are summarized as the mean±SD. Student t test or two-way ANOVA is used to determine statistically significant differences between treatment groups, with Bonferroni's correction for multiple comparisons when appropriate (Prism 6: GraphPad Software). Survival analysis is performed using the Kaplan-Meier method and the Mantel-Cox log rank test is applied (Prism 6: GraphPad Software). All P values less than 0.05 are considered statistically significant.

### Results:

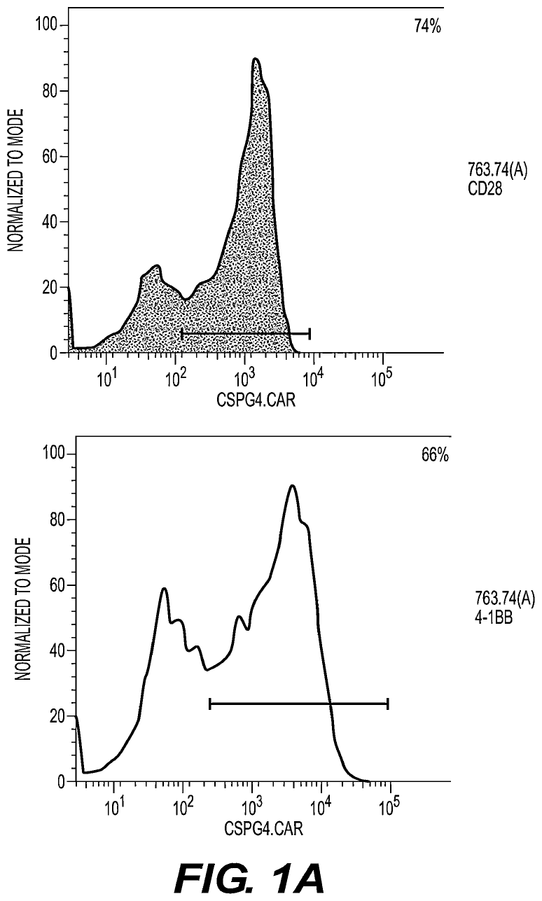

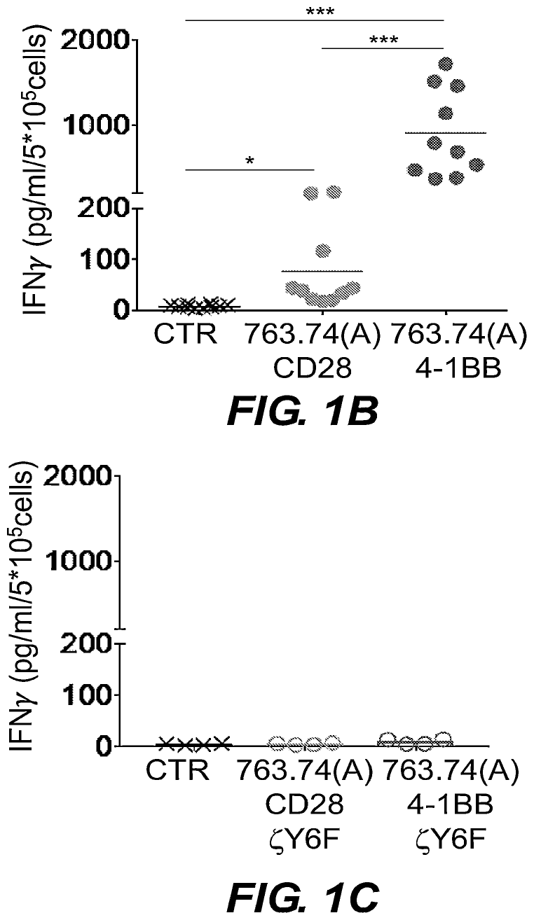

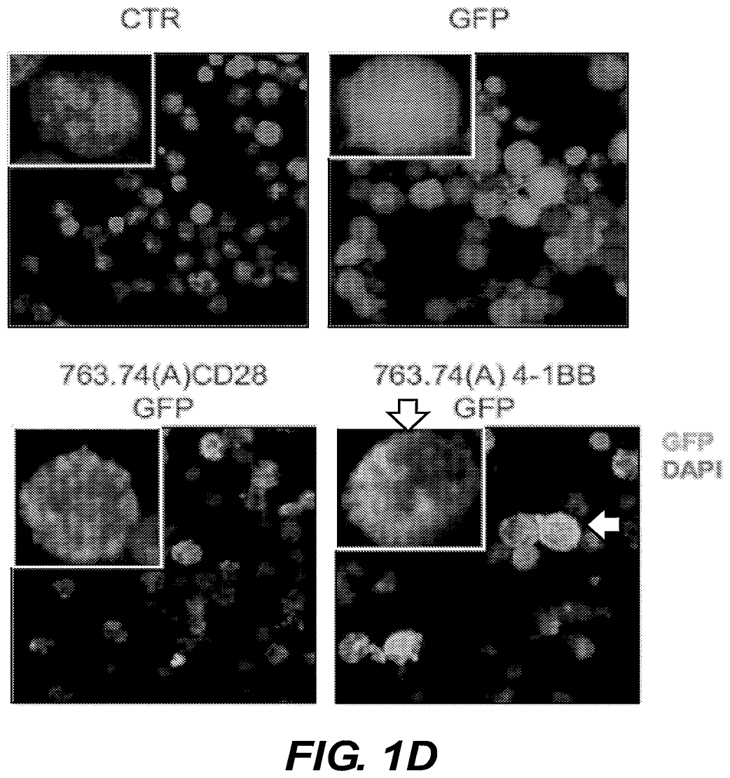

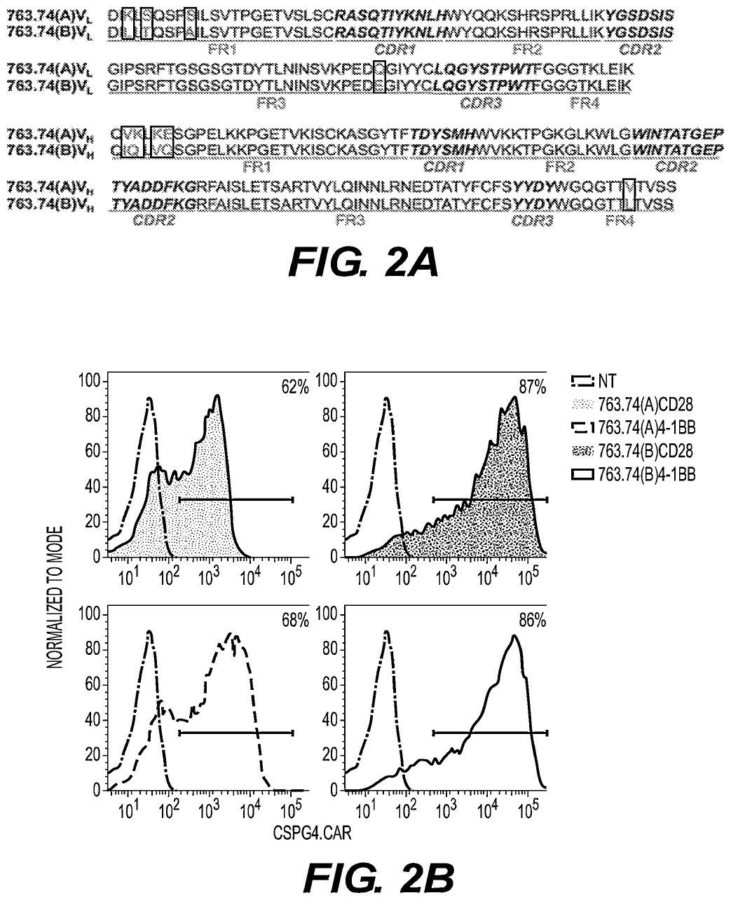

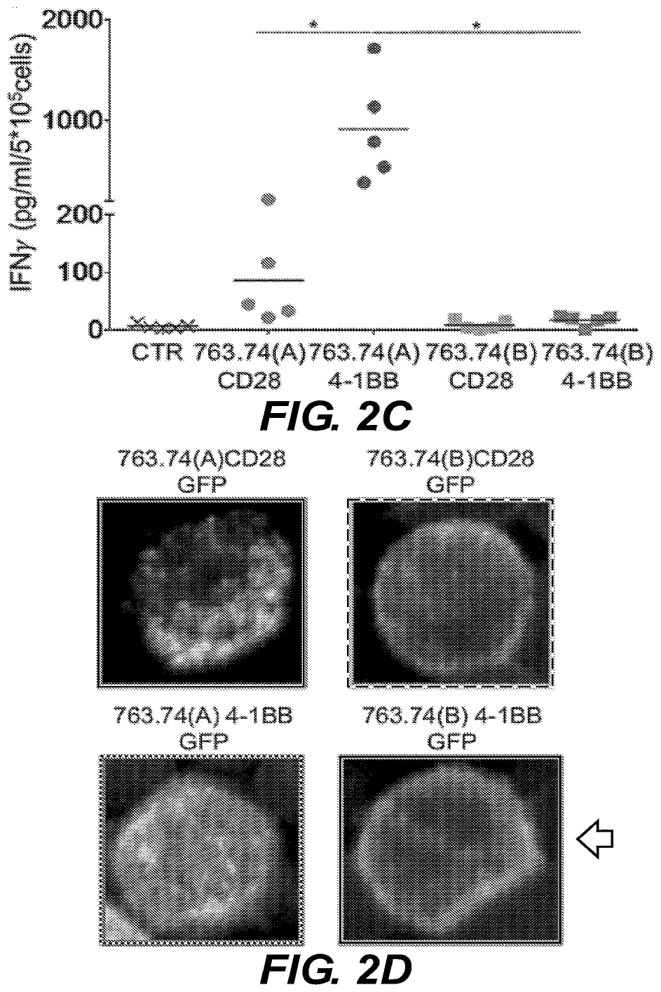

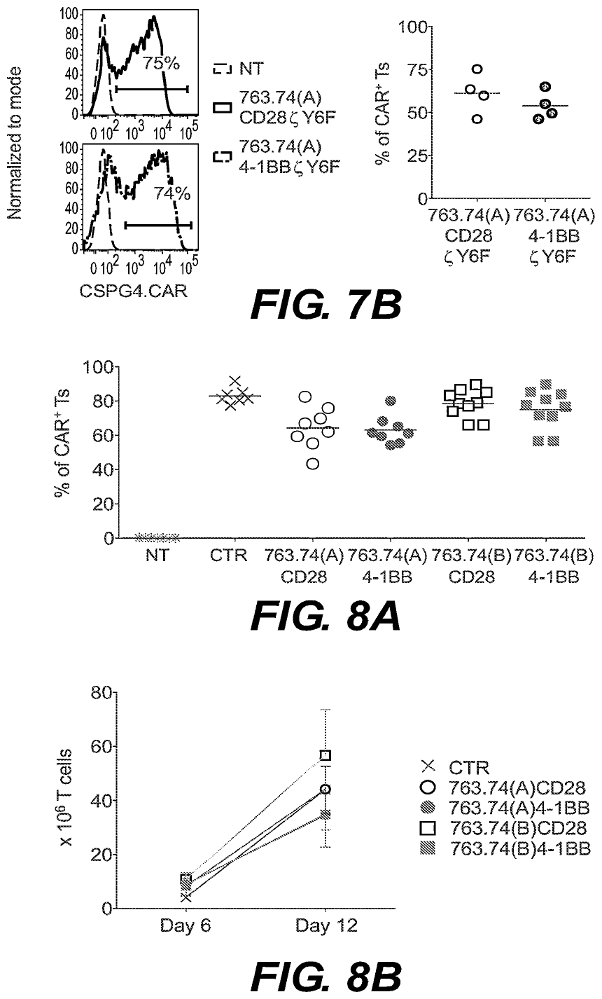

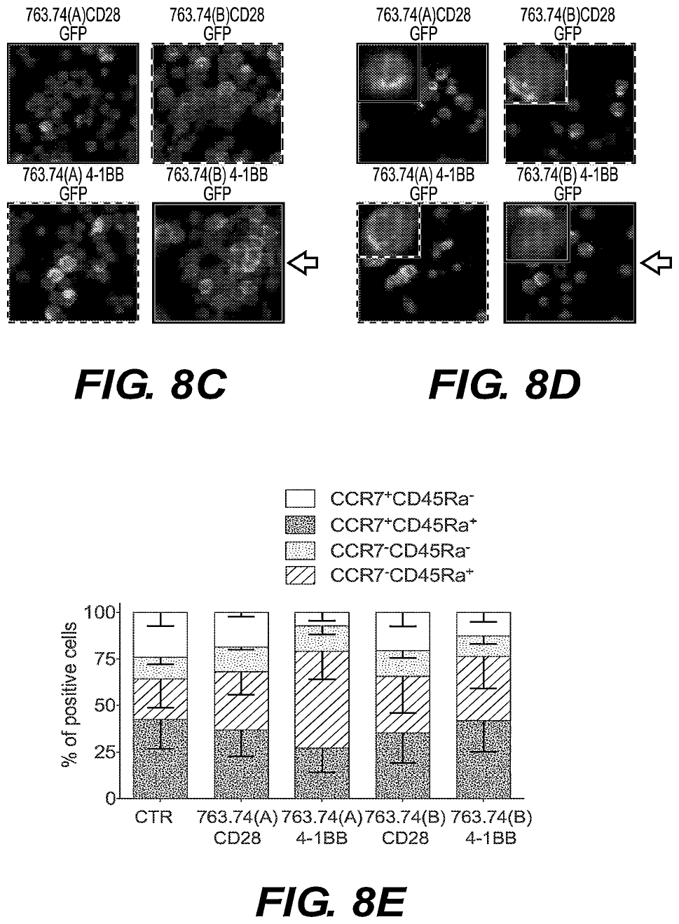

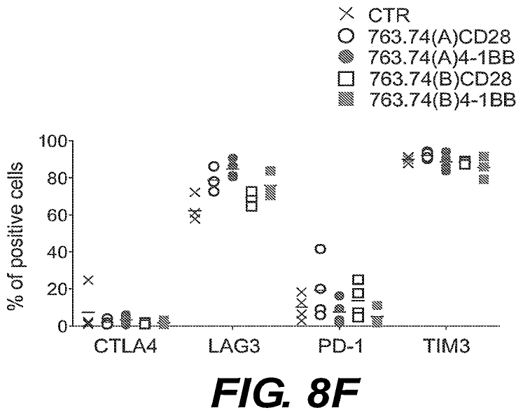

Amino acid substitutions within the Frame Work Regions (FWRs) of the scFv abrogate CAR tonic signaling. CAR targets the chondroitin sulphate proteoglycan 4 (CSPG4) antigen in which the antigen binding moiety is the scFv 763.74(A) obtained from the 763.74 murine monoclonal antibody (mAb) targeted tumor cells expressing CSPG4 both in vitro and in vivo. See Pellegatta et al., (2018) and Geldres et al., “T lymphocytes redirected against the chondroitin sulfate proteoglycan-4 control the growth of multiple solid tumors both in vitro and in vivo,” Clin. Cancer Res. 20:962-971 (2014). However, T cells expressing the scFv 763.74(A) CAR (FIG. 1A) encoding either CD28 or 4-1BB costimulatory endodomains show release of IFNγ in the absence of antigen stimulation, a phenomenon defined as CAR tonic signaling (FIG. 1B). See Long et al. (2015). Spontaneous IFNγ release by T cells expressing the scFv 763.74(A) CARs is strictly dependent on CAR signaling because mutations of the tyrosine of the immunoreceptor tyrosine-based activation motifs (ITAMs) of the CAR-CD3γ chain that prevent tyrosine phosphorylation completely abrogate the spontaneous IFNγ release (FIG. 1C and FIG. 7A,B). To study the distribution of CAR molecules on the cell surface of T cells, scFv 763.74(A) CARs are generated in which the CD3ζ chain of the CAR is fused at COOH terminal with GFP. Using confocal microscopy imaging, the scFv 763.74(A) CARs are shown to form membrane clusters in the absence of CAR crosslinking likely indicating self-aggregation of CAR molecules (FIG. 1D). The sequence of the scFv 763.74(A) is obtained from an early passage of the hybridoma 763.74 secreting the murine IgG1 mAb, which recognizes a peptide epitope of the human CSPG4. See Reinhold et al., “Specific lysis of melanoma cells by receptor grafted T cells is enhanced by anti-idiotypic monoclonal antibodies directed to the scFv domain of the receptor,” J Invest Dermatol. 112:744-750 (1999). It is well established that culture passages of hybridoma affect the growth rate of the hybridoma and the yield of the secreted antibody. See Correa et al., “Effects of passage number on growth and productivity of hybridoma secreting MRSA anti-PBP2a monoclonal antibodies,” Cytotechnology 68:419-427 (2016). It has also been described that upon culture passages amino acid substitutions may occur in both CDRs and FWRs in subclones derived from the hybridoma. See, Xin and Cutler,“Hybridoma passage in vitro may result in reduced ability of antimannan antibody to protect against disseminated candidiasis,” Infect. Immun. 74:4310-4321 (2006). In light of this possibility, the V_{L} and V_{H} domains of a late passage of the 763.74 hybridoma are sequence. We obtained two V_{L} and V_{H} sequences in which amino acid substitutions were identified in the FWRs (FR1 and FR3) of both V_{L} and V_{H} (FIG. 2A). A new scFv called scFv 763.74(B) is assembled, a new scFv 763.74(B) CAR is generated and the resulting sequences compared side-by-side with scFv 763.74(A) CARs for evidence of tonic signaling. All CARs are equally expressed in T cells (FIG. 2B and FIG. 8A), and CAR-T cells equally expanded in vitro (FIG. 8B). However, T cells expressing scFv 763.74(B) CAR encoding either CD28 or 4-1BB costimulatory endodomains did not show spontaneous release of IFNγ (FIG. 2C). A more homogeneous distribution of the scFv 763.74(B) CARs on the membrane of T cells using confocal microscopy of GFP-tagged CARs is observed (FIG. 1D and FIG. 8C). Of note, cross-linking of CARs expressed in T cells mediated by an anti idyotipic mAb (MK2-23 mAb) caused significant cluster formation of CAR molecules regardless the type of scFv expressed, further indicating that clusters identified by confocal microscopy truly reflect the formation of CAR aggregates (FIG. 8D). Phenotypic analysis of T cells expressing scFv 763.74(A) CARs or scFv 763.74(B) CARs do not show differences in the expression of memory and exhaustion markers indicating that tonic signaling may not induce an exhaustion phenotype during the 10-14 days of culture usually required to manufacture CAR-T cells for clinical use (FIG. 8E,F). see Ramos et al., “Clinical responses with T lymphocytes targeting malignancy-associated kappa light chains,” J. Clin. Invest 126:2588-2596 (2016); Ramos et al., “Clinical and immunological responses after CD30-specific chimeric antigen receptor-redirected lymphocytes,” J Clin. Invest 127:3462-3471 (2017). Overall, these data indicate that amino acid substitutions within the FWRs of a scFv antibody are sufficient in causing self-aggregation of the scFv in the CAR format and tonic signaling in T cells defined as basal release of IFNγ.

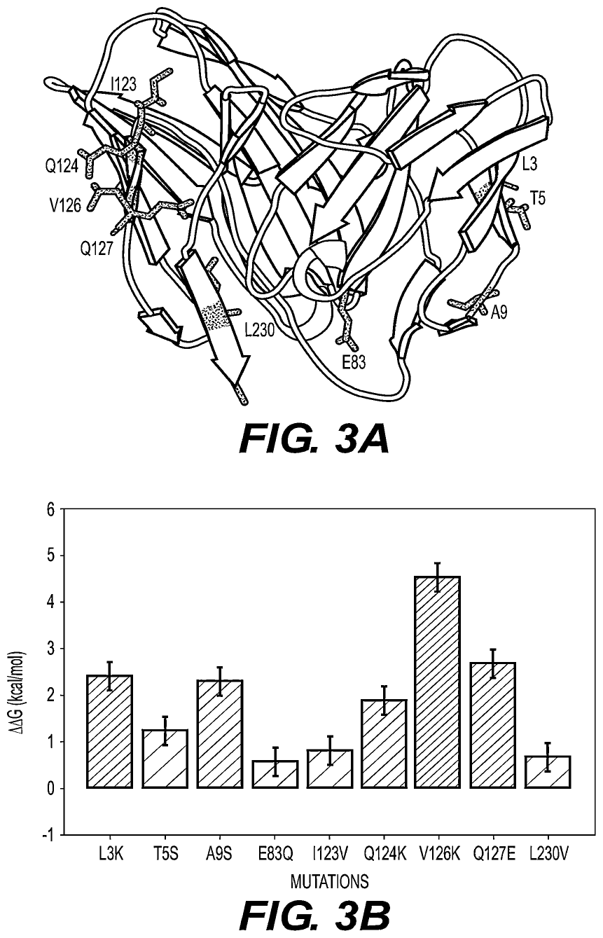

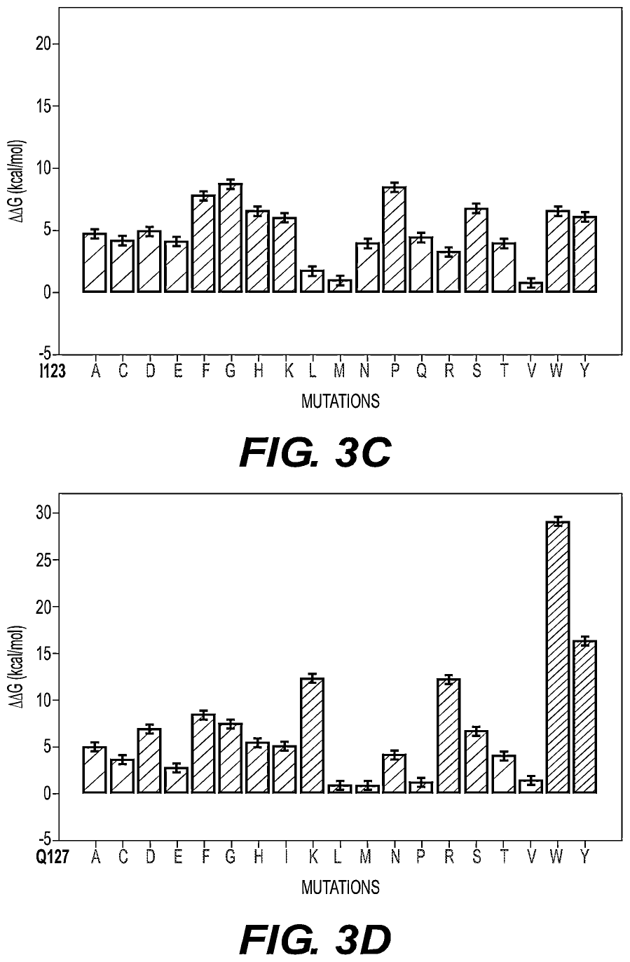

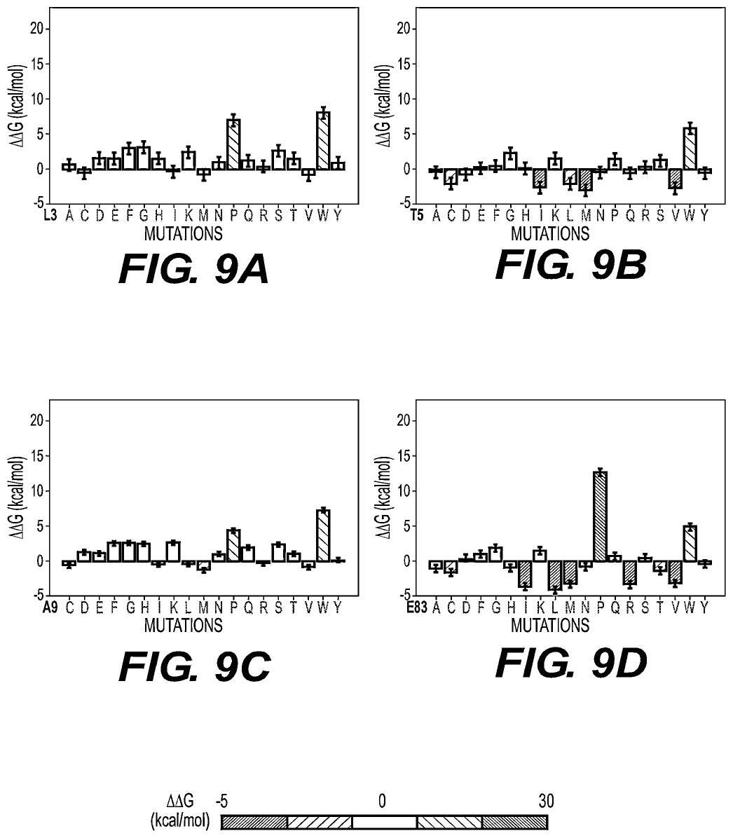

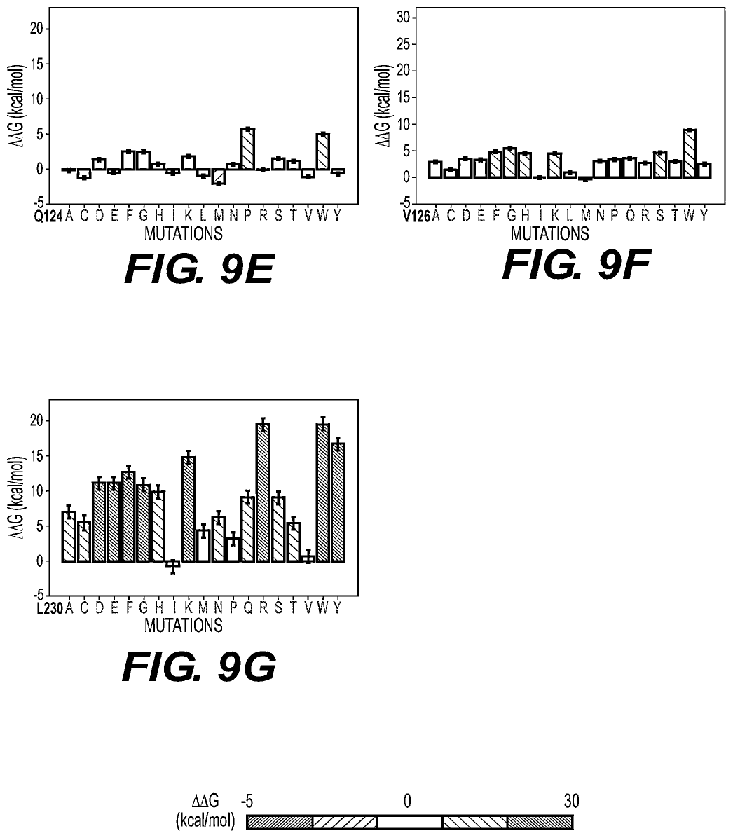

Amino acid substitutions within the FWRs of the scFv cause protein destabilization. To study if differences in amino acids between the scFv 763.74(A) and scFv 763.74(B) cause destabilization of the scFv, the 3D conformation of the scFv 763.74(B) through homology modeling and optimized the structure for in silico mutagenesis (FIG. 3A) are generated. The Eris tool is employed to delineate the effect of FWR mutations, L3K, TSS, A9S, E83Q, I123V, Q124K, V126K, Q127E, and L230V on the structure of the scFv 763.74(B). Eris estimated the ΔΔGₘᵤₜ for the above-mentioned mutations as 2.41, 1.26, 2.29, 0.47, 0.79, 1.87, 4.58, 2.70, and 0.67 kcal/mol, respectively (FIG. 3B). Specifically, the mutations are destabilizing scFv 763.74(A) structure (ΔΔGₘᵤₜ>0) and subsequently affecting the CAR spontaneous aggregation. Further, to cross-validate the structural conformation of the scFv 763.74(B), Eris analysis is performed to identify stabilizing mutations (ΔΔGₘᵤₜ<0) at the FWR mutated sites. The analysis indicates that mutations such as E83L, E83I, TSM, and Q124M result in negative ΔΔGₘᵤₜ, notifying their potential stabilizing capability (FIG. 9). Furthermore, residues such as 1123 and Q127 are identified as critical for the stability of the scFv 763.74(B) structure. The substitution of any of the other 19 amino acids at 1123 or Q127 positions highly destabilizes the scFv 763.74(B) structure (ΔΔG>0) and thereby affects the protein aggregation (FIG. 3C). Overall, these data indicate that amino acid substitutions within the FRWs of a scFv mAb destabilize the protein and cause self-aggregation of the scFv, and that computation guided analyzes can be used to stabilize the protein structure.

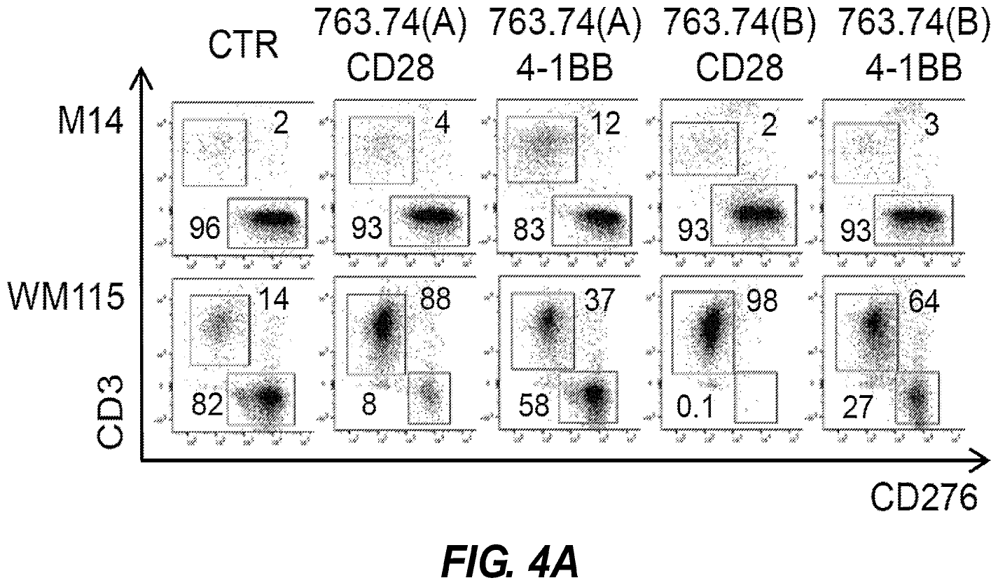

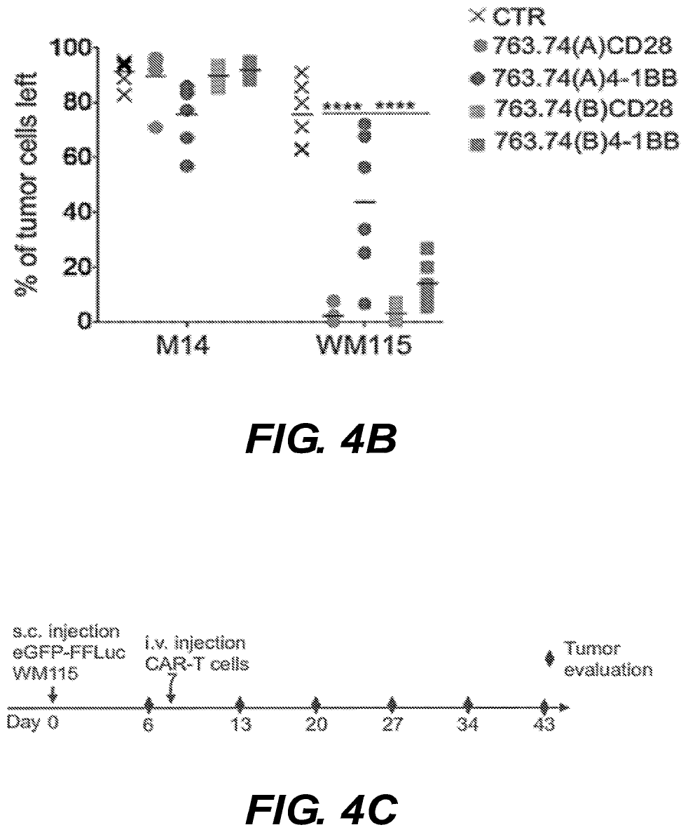

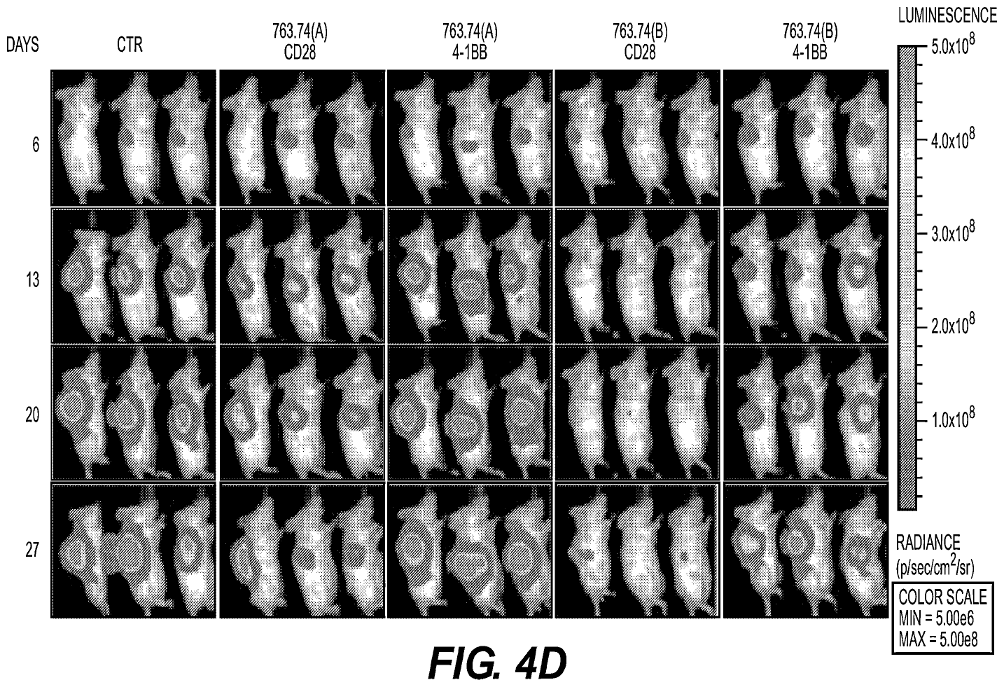

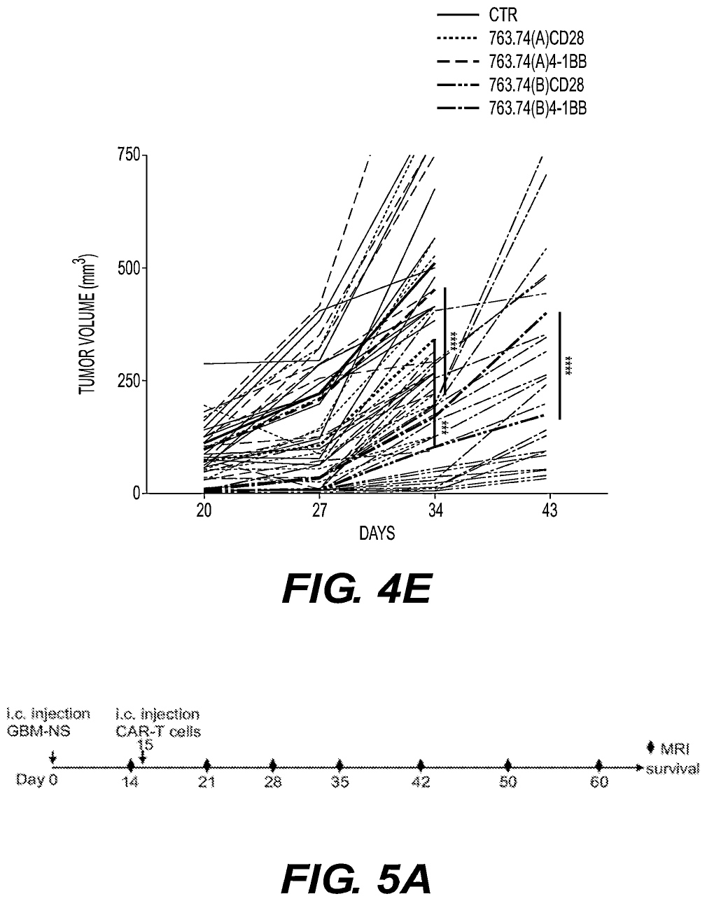

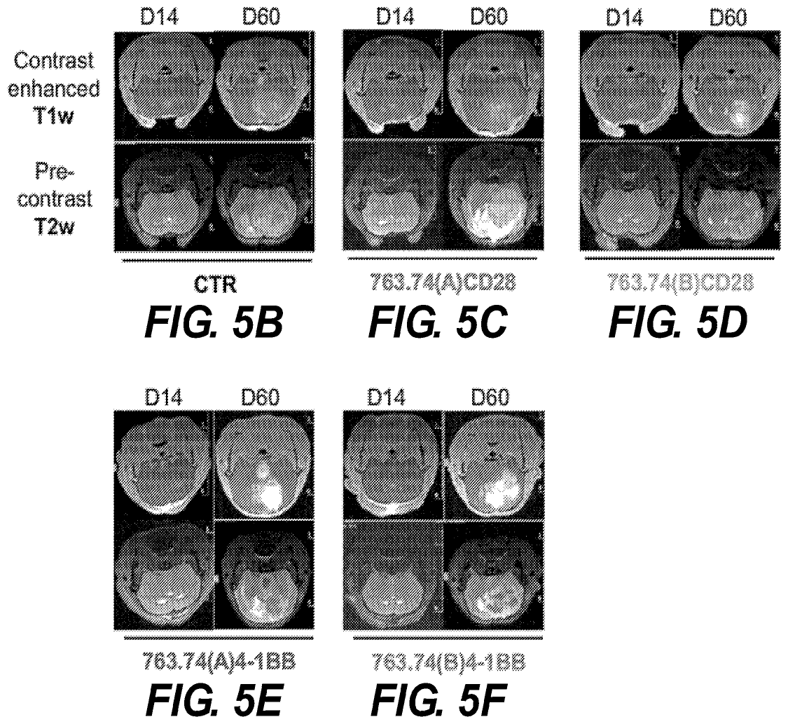

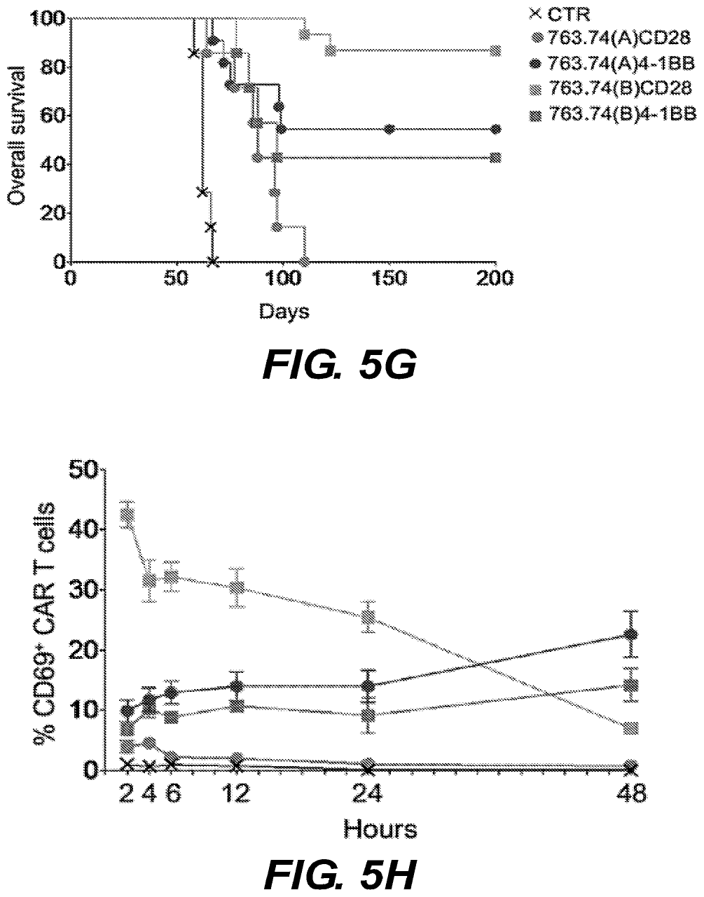

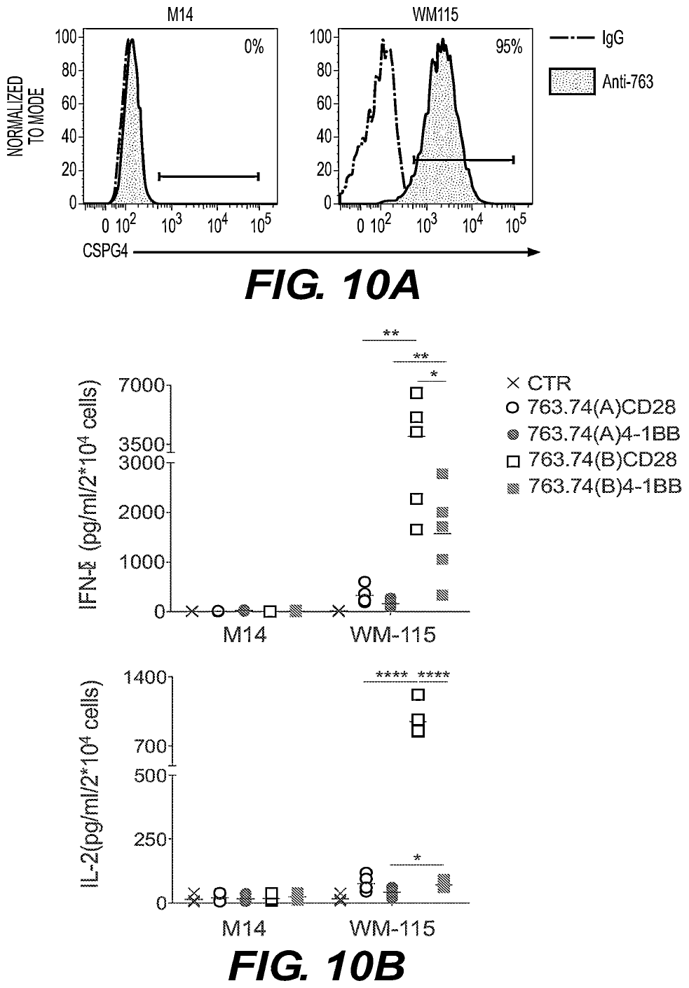

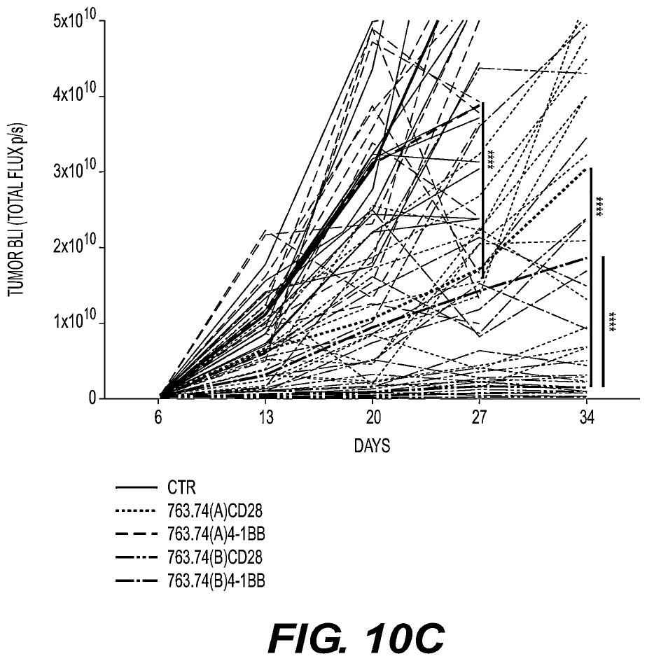

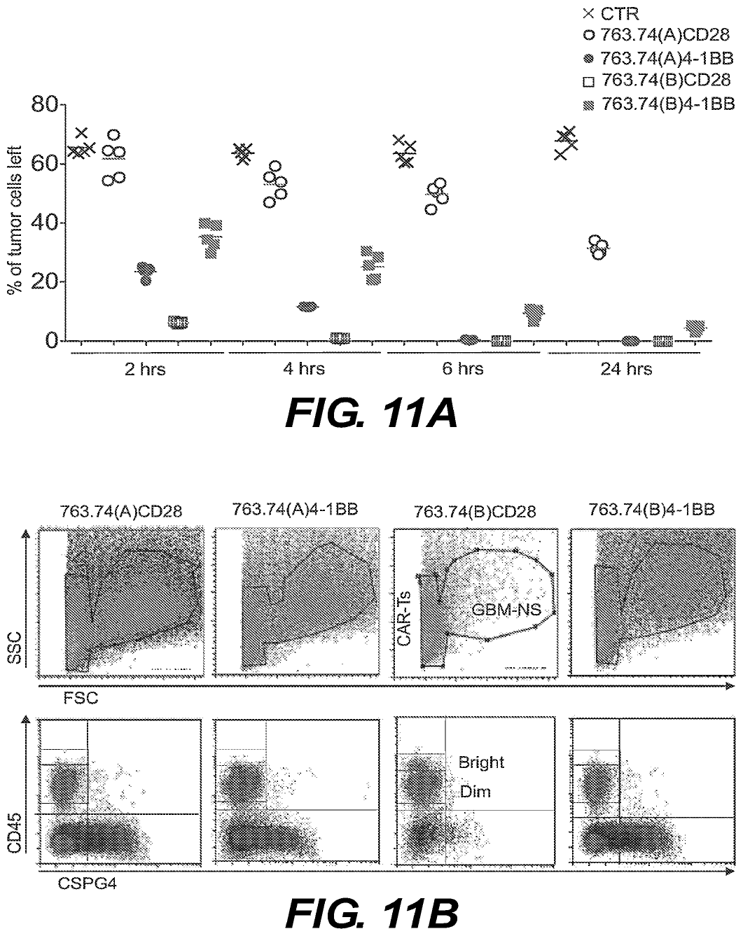

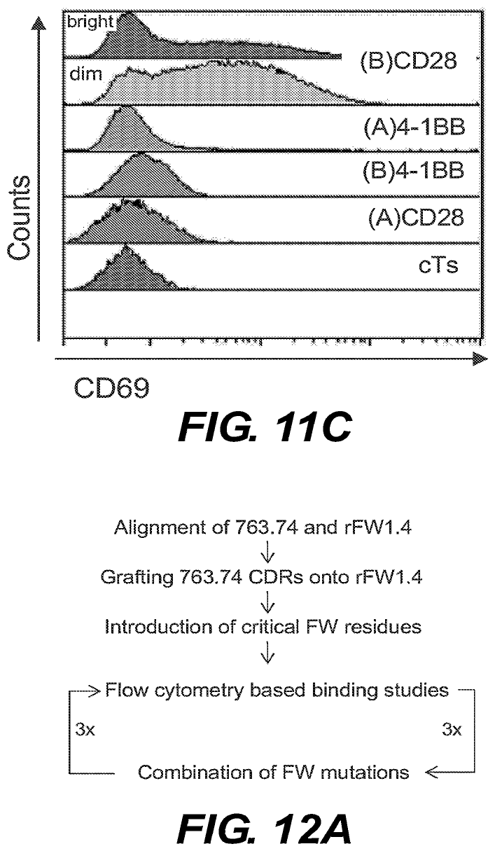

Amino acid substitutions within the FWRs of the scFv enhance functions of CAR-T cells. The effect of amino acid substitutions within the FWRs of the scFv affect the anti-tumor activity of CAR-T cells on the melanoma cell lines WM115 (CSPG4+) and M14 (CSPG4−) (FIG. 10A). Paralleling the spontaneous release of IFNγ, T cells expressing the 763.74(A) CAR with 4-1BB showed compromised capacity to eliminate tumor cells in vitro (residual tumor cells 43.6%±26.0%) at the end of a 4 day coculture when CAR-T cells and tumor cells are plated at the 1 to 5 ratio (FIG. 4A,B). In contrast, CD28 costimulation seemed to allow complete tumor elimination for both 763.74(A) and 763.74(B) CARs (residual tumor cells 2.2%±2.7% and 2.9%±2.6%, respectively). Of note, T cells expressing the 763.74(B) CAR with 4-1BB costimulation, shows improved antitumor effects as compared to T cells expressing 763.74(A) CAR with 4-1BB, but does not completely eliminate the tumor cells (residual tumor cells 14.1%±8.0% and 43.6%±26.0%, respectively) (FIG. 4A,B). CAR-T cells does not eliminate the melanoma cell line M14 that lacks CSPG4 expression indicating that antigen specificity is not affected by amino acid substitutions within the FWRs. Of note, only T cells expressing the 763.74(B) CARs consistently released detectable amounts of Thl cytokines in the culture supernatant with WM115 tumor cells (FIG. 10B). The superior antitumor effects of 763.74(B) CAR-T cells is more evident in vivo using the eGFP-FFLuc WM115 xenogeneic NSG mouse model (FIG. 4C). T cells expressing the 763.74(B) CAR with CD28 exhibit the most prominent antitumor effects measured as both tumor bioluminescence (FIG. 4D and FIG. 10C) and tumor size (FIG. 4E). Enhanced functions of CAR-T cells expressing the 763.74(B) CAR are confirmed in our previously described glioblastoma (GBM) tumor model in which nude mice are engrafted in the brain with primary GBM-derived neurospheres (GBM-NS) and treated via intratumor inoculation of CAR-T cells. See Pellegatta et al., “Constitutive and TNFalpha-inducible expression of chondroitin sulfate proteoglycan 4 in glioblastoma and neurospheres: Implications for CAR-T cell therapy,” Sci. Transl. Med. 10 (2018) (“Pellegatta et al. 2018”); see FIG. 5A. In this model tumor engraftment and progression are monitored by magnetic resonance imaging (MRI). Rapid tumor progression in mice treated with control T cells or T cells expressing the 763.74(A) CAR encoding CD28 is observed, and in these animals tumor masses occupy the whole hemispheres and infiltrate the contralateral one (FIG. 5B,C). Mice treated with T cells expressing the 763.74(B) CAR encoding CD28 showed the most evident antitumor effects as indicated by smaller and more circumscribed lesions (FIG. 5D), but tumor control is also observed in mice treated with T cells expressing either 763.74(A) or 763.74(B) CAR encoding 4-1BB even if the antitumor effects are less dramatic (FIG. 5E,F). T cells expressing either 763.74(A) or 763.74(B) CARs encoding 4-1BB prolonged survival as compared to mice treated with control T cells (p<0.0001). However, T cells expressing the 763.74(B) CAR encoding CD28 were the most effective in prolonging survival (p<0.0001 vs. CTR; p=0.04 vs. 763.74(A) with 4-1BB, p=0.01 vs. 763.74(B) with 4-1BB). Modest activity of T cells expressing the 763.74(A) CAR encoding CD28 is observed since no mice survive more than 110 days. Pellegatta et al. 2018 (FIG. 5G). To further characterize the remarkable antitumor effects of T cells expressing the 763.74(B) CAR encoding CD28 in this GBM model, the activation status of T cells immediately after intracranial infusion is investigate. Tumor masses are explanted 2, 4, 6, 12, 24 and 48 hours after CAR-T cell infusion. T cells expressing the 763.74(B) CAR with CD28 upregulated CD69 within 2 hours after inoculation (42.5±2.1% CD69⁺ T cells) are observed, and maintained high CD69 levels for 24 hours (22.5±1.5% CD69⁺ T cells), which is consistent with previous reports indicating the fast activity of CAR-T cells encoding the CD28 endodomain. See Zhao et al. “Structural Design of Engineered Costimulation Determines Tumor Rejection Kinetics and Persistence of CAR T Cells. Cancer Cell 28:415-428 (2015); Sun et al. 2020. (FIG. 5H). Additional experiments in vitro further demonstrate the rapid antitumor effects of T cells expressing the 763.74(B) CAR with CD28 (FIG. 11).

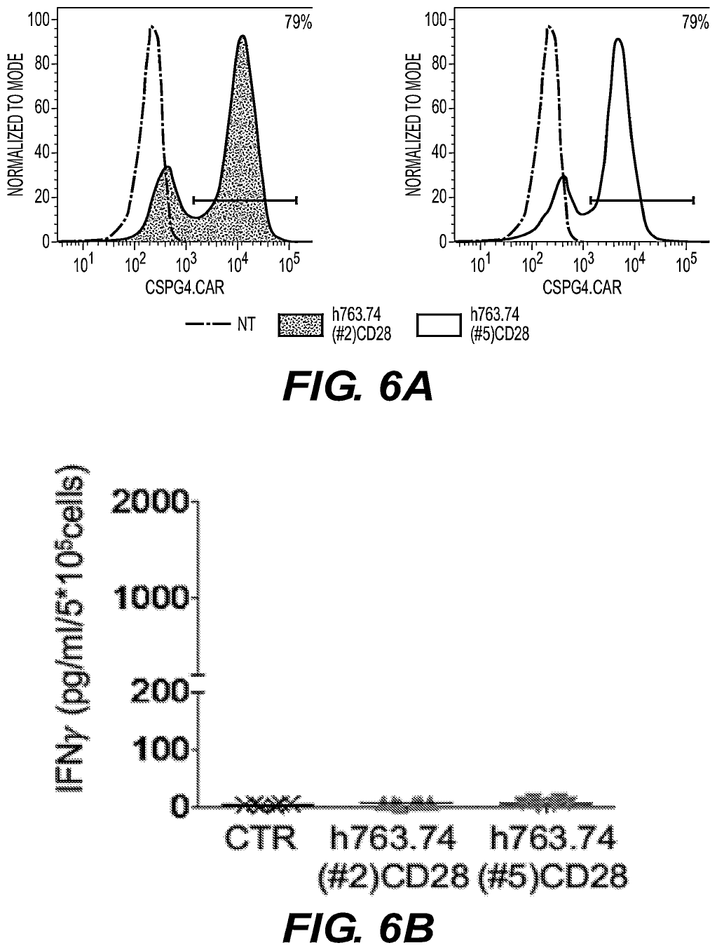

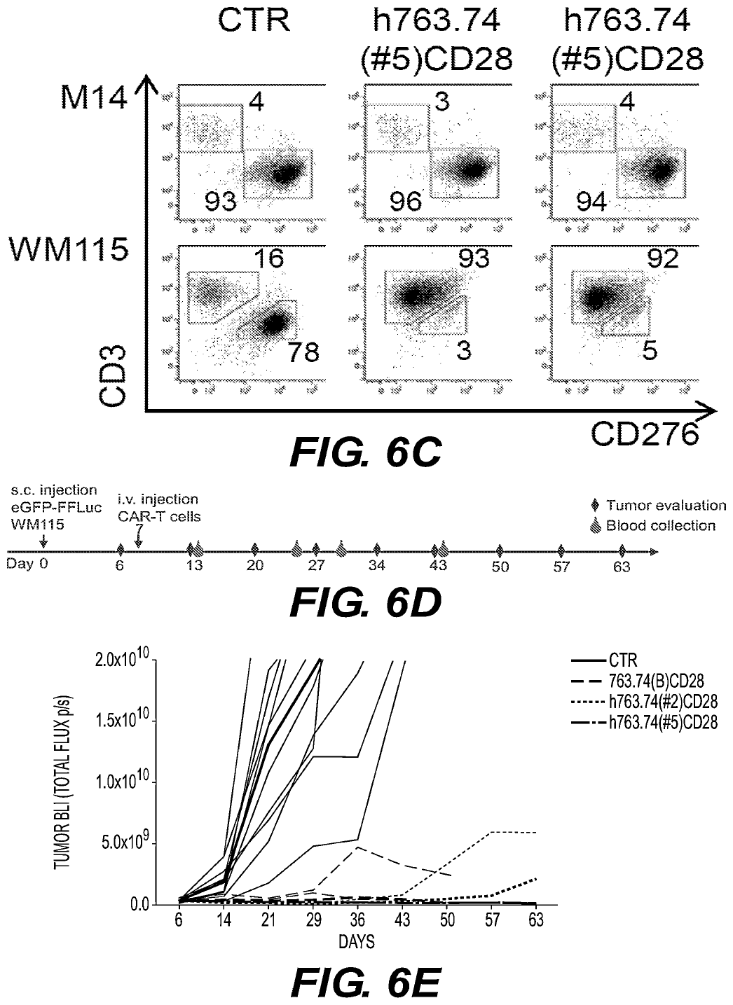

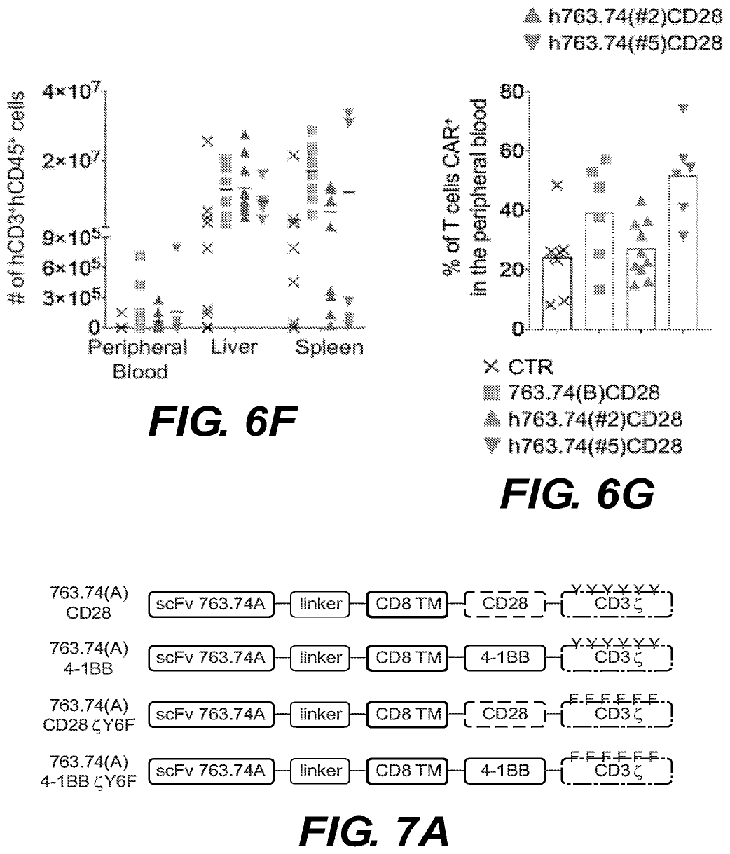

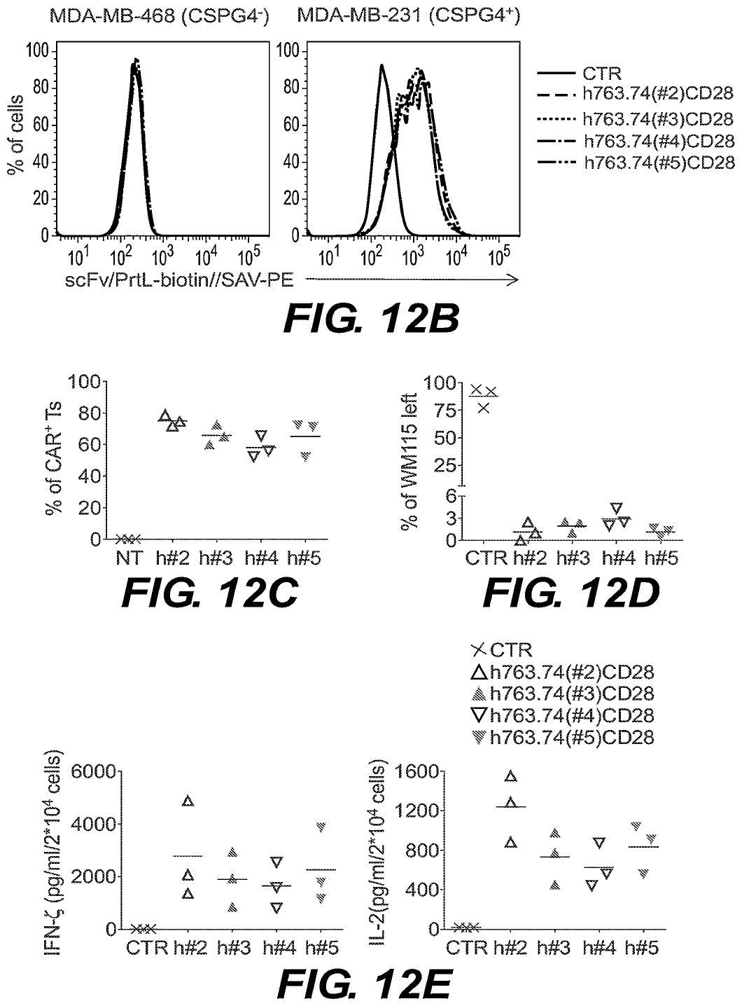

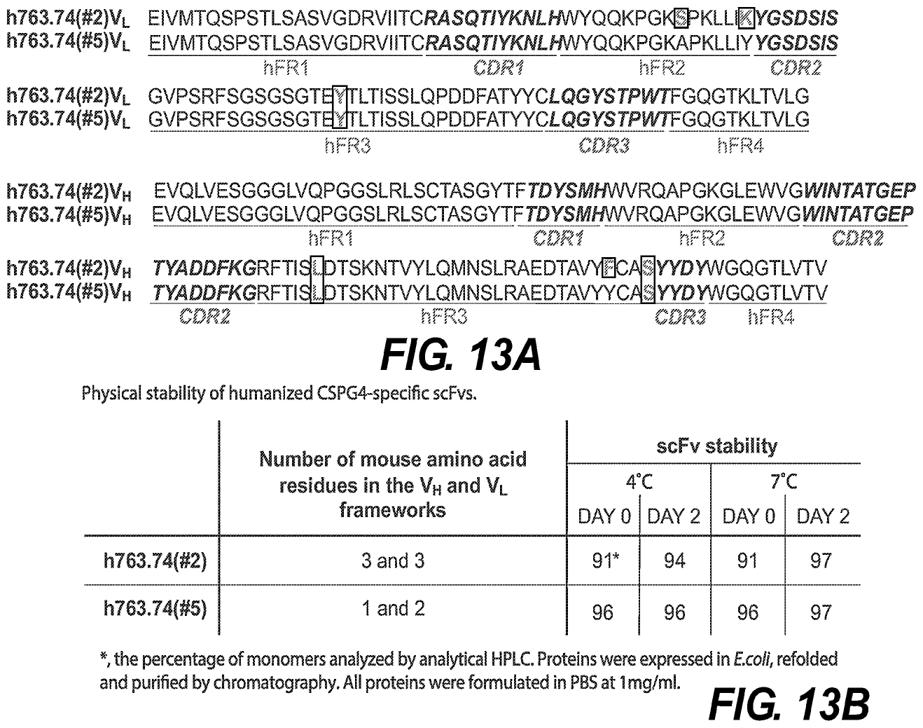

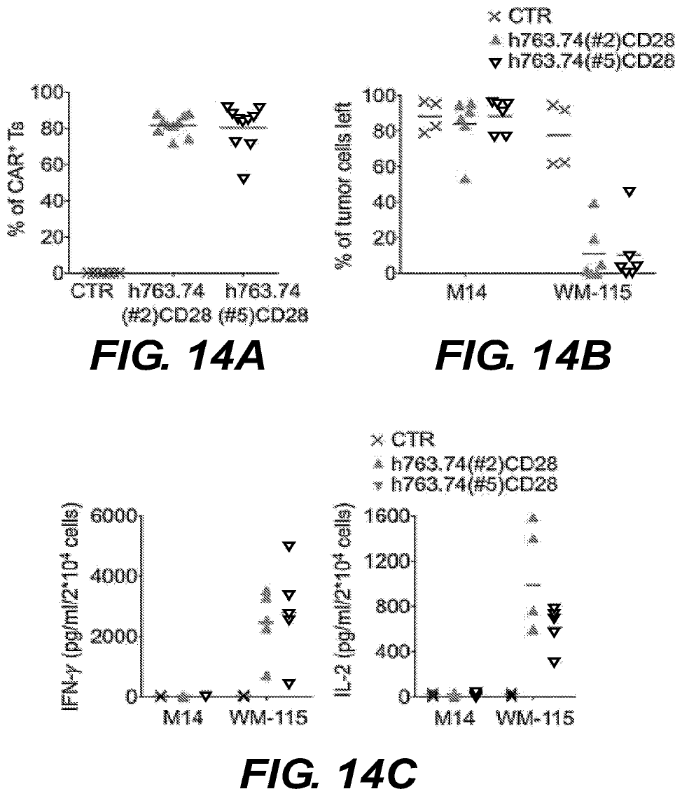

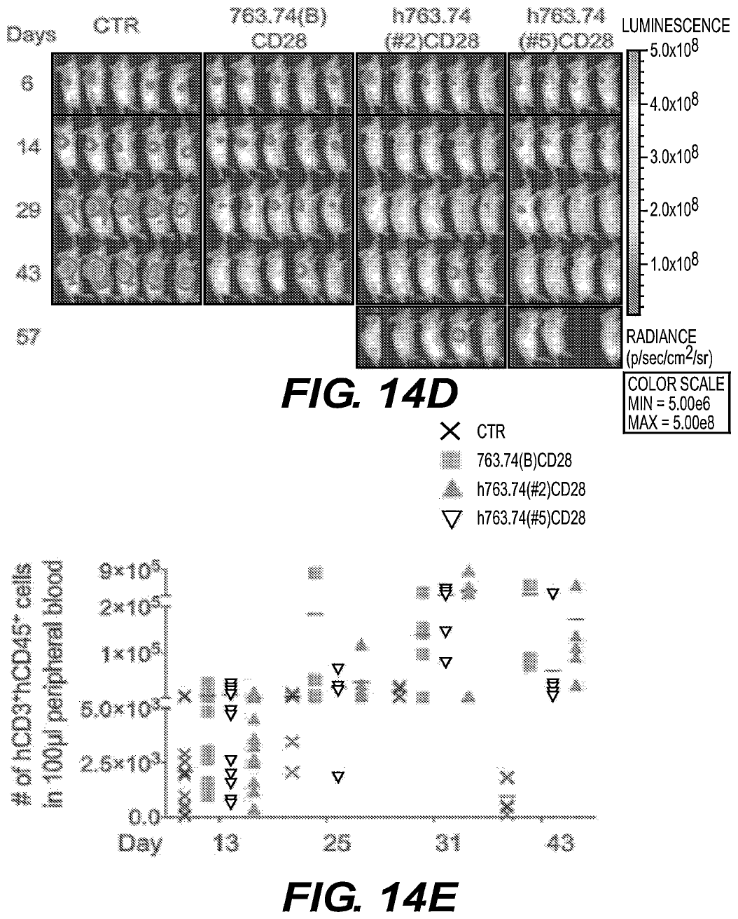

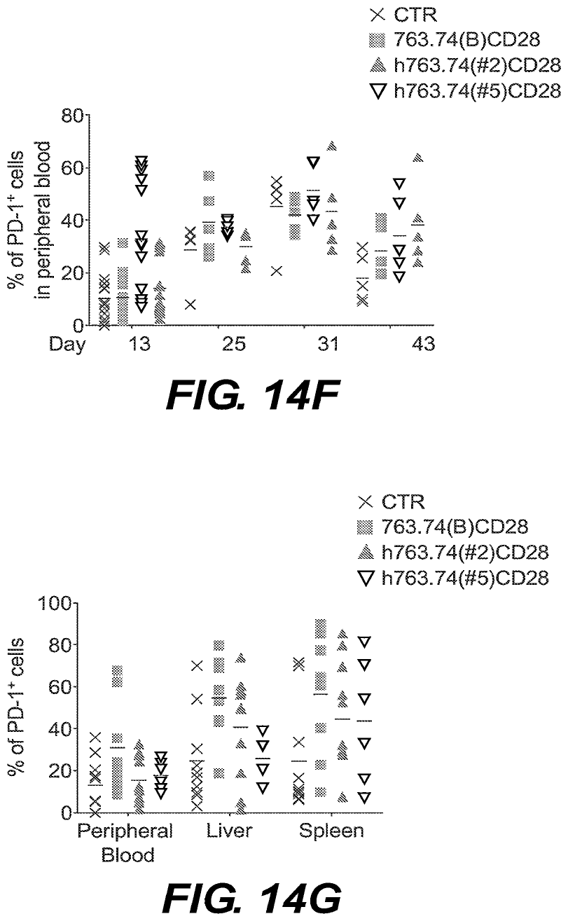

Humanization of the FWRs of the scFv abrogates CAR tonic signaling. Humanization of murine derived scFvs is a strategy proposed to prevent humoral and T cell responses to the CAR. See Sun et al., “Construction and evaluation of a novel humanized HER2-specific chimeric receptor,” Breast Cancer Res. 16:R61 (2014); Johnson et al., “Rational development and characterization of humanized anti-EGFR variant III chimeric antigen receptor T cells for glioblastoma,” Sci. Transl. Med. 7:275ra22 (2015). Substituting the murine FWRs of the scFv with human FWRs could be used to abrogate the CAR tonic signaling. The sequence of 763.74(A) and the stable human framework rFW1.4 were aligned, and the critical amino acids were identified. See Ewert et al., “Stability improvement of antibodies for extracellular and intracellular applications: CDR grafting to stable frameworks and structure-based framework engineering,” Methods 34:184-199 (2004). One CDR graft with no mutations in the rFW1.4 sequence and seven variants with up to 24 mutations in the critical regions were designed in the first round of engineering (FIG. 12A). Humanized scFv variants alone (i.e. only extracellular domain of CARs) and the wild-type murine scFv were expressed in E. coli, refolded and purified by size exclusion chromatography (SEC). The wild-type murine scFv variant was not refoldable due to aggregation. Therefore, we were unable to purify it in a soluble form. This result further suggests that the murine 763.74(A) scFv is unstable. A humanized variant with no mutations in the rFW1.4 is expressed, but does not bind CSPG4. Nearly all other humanized scFv variants are successfully expressed and able to bind CSPG4⁺ cells. In the next engineering rounds, humanized variants with the minimal number of 763.74(A) murine FW residues are further subjected to chain shuffling of the V_{H} and V_{L}. A total of 26 humanized scFvs are produced. Four humanized scFvs (h763.74 #2, h763.74 #3, h763.74 #4 and h763.74 #5) with the minimal number of murine FWR residues and retained CSPG4-binding activity are selected for further studies (FIG. 12B). CARs with all four humanized scFv 763.74(A) with CD28 endodomain are generated and in vitro coculture experiments with tumor cells are performed. T cells expressing h763.74.CAR #2 and h763.74.CAR #5 showed a trend for better antitumor activity and higher production of IFNγ and IL-2 in vitro and are selected for further studies (FIG. 12C-E). To characterize the two selected humanized scFvs h763.74.CAR #2 and h763.74.CAR #5 (FIG. 13A), storage stability studies with purified soluble scFvs are performed. Proteins are prepared at 1 mg/ml and stored for 48 hours at 4° C. and 37° C. After incubation, samples are analyzed by SEC to estimate the percentage of monomeric proteins. Under tested conditions, no detectable protein loss is observed, and the percentage of monomers remained above 91%-97% (FIG. 13B). This method demonstrates that all four selected humanized scFvs are monomeric under tested conditions and do not dimerize or aggregate upon storage at 4° C. and 37° C. The expression of h763.74.CAR #2 and h763.74.CAR #5 in T cells is adequate (FIG. 6A and FIG. 14A), and T cells do not show spontaneous release of IFNγ (FIG. 6B). T cells expressing the h763.74.CAR #2 and h763.74.CAR #5 successfully controlled the CSPG4⁺ WM115 melanoma cell growth in vitro (residual tumor cells 11%±15% and 10%±17% respectively), while they do not target the CSPG4⁻ M14 melanoma cell line indicating that antigen specificity is maintained (FIG. 6C and FIG. 14B). The anti-tumor activity of T cells expressing h763.74.CAR #2 and h763.74.CAR #5 is corroborated by specific production of IFNγ and IL-2 (FIG. 14C). T cells expressing the h763.74.CAR #2 and h763.74.CAR #5 are compared with T cells expressing the 763.74(B) CAR encoding the CD28 endodomain in the xenogeneic WM115 melanoma mouse model (FIG. 6D). T cells expressing the h763.74.CAR #2 and h763.74.CAR #5 show potent antitumor activity (FIG. 6E and FIG. 14D). Furthermore, T cells are detectable in the peripheral blood of treated mice at different time points (FIG. 14E), and in the liver and spleen at the time of euthanasia (FIG. 6F), and T cells retain the CAR expression (FIG. 6G). Of note, T cells expressing h763.74.CAR #2 and h763.74.CAR #5 do not show increased expression of PD-1 as compared to 763.74(B) CAR with CD28 (FIG. 14F,G). Overall, these data show that the humanization of a scFv can be used to eliminate tonic signaling of CAR molecules maintaining specific antitumor effects.

1. A binding member having a binding specificity to chondroitin sulfate proteoglycan 4 (CSPG4), said binding member comprises
a variable light chain sequence comprising light chain complementarity determining regions (“LCDRs”) LCDR1 to LCDR3 sequences set forth in SEQ ID NOs: 1-3 and
a light chain framework sequence 1 (VL-FR1) selected from the group consisting of SEQ ID NOs:153 to 221;
a light chain framework sequence 2 (VL-FR2) selected from the group consisting of SEQ ID NOs:222 to 225;
a light chain framework sequence 3 (VL-FR3) selected from the group consisting of SEQ ID NOs:226 to 249; and
a light chain framework sequence 4 (VL-FR4) selected from the group consisting of SEQ ID NOs:250 to 255;
a variable heavy chain sequence comprising heavy chain complementarity determining regions (“HCDRs”) HCDR1 to HCDR3 sequences set forth in SEQ ID NOs: 4 to 6 and,
a heavy chain framework sequence 1 (VH-FR1) selected from the group consisting of SEQ ID NOs:256 to 349;
a heavy chain framework sequence 2 (VH-FR2) selected from the group consisting of SEQ ID NOs:350 to 353;
a heavy chain framework sequence 3 (VH-FR3) selected from the group consisting of SEQ ID NOs:354 to 360; and
a heavy chain framework sequence 4 (VH-FR4) selected from the group consisting of SEQ ID NOs:361 to 382.

2. The binding member having a binding specificity to CSPG4 of claim 1, wherein said light chain framework sequence 1 (VL-FR1) is selected from the group consisting of SEQ ID NOs:153 to 158;
said light chain framework sequence 2 (VL-FR2) is selected from the group consisting of SEQ ID NOs:222 to 225;
said light chain framework sequence 3 (VL-FR3) is selected from the group consisting of SEQ ID NOs:226 to 231; and
said light chain framework sequence 4 (VL-FR4) is selected from the group consisting of SEQ ID NOs:250 to 255;
said heavy chain framework sequence 1 (VH-FR1) is selected from the group consisting of SEQ ID NOs:347 to 349;
said heavy chain framework sequence 2 (VH-FR2) is selected from the group consisting of SEQ ID NOs:350 to 353;
said heavy chain framework sequence 3 (VH-FR3) is selected from the group consisting of SEQ ID NOs:354 to 360; and
said heavy chain framework sequence 4 (VH-FR4) is selected from the group consisting of SEQ ID NOs:361 to 362.

3. The binding member having a binding specificity to CSPG4 of claim 1, wherein said variable light chain sequence comprises a sequence selected from the group consisting of SEQ ID NOs: 67 to 109 and said variable heavy chain sequence comprises a sequence selected from the group consisting of SEQ ID NOs: 110 to 152.

4. A nucleic acid encoding a polypeptide comprising a variable light chain sequence comprising light chain complementarity determining regions (“LCDRs”) LCDR1 to LCDR3 sequences set forth in SEQ ID NOs: 1-3 and
a light chain framework sequence 1 (VL-FR1) selected from the group consisting of SEQ ID NOs:153 to 221;
a light chain framework sequence 2 (VL-FR2) selected from the group consisting of SEQ ID NOs:222 to 225;
a light chain framework sequence 3 (VL-FR3) selected from the group consisting of SEQ ID NOs:226 to 249; and
a light chain framework sequence 4 (VL-FR4) selected from the group consisting of SEQ ID NOs:250 to 255.

5. A nucleic acid encoding a polypeptide comprising a variable heavy chain sequence comprising heavy chain complementarity determining regions (“HCDRs”) HCDR1 to HCDR3 sequences set forth in SEQ ID NOs: 4 to 6 and
a heavy chain framework sequence 1 (VH-FR1) selected from the group consisting of SEQ ID NOs:256 to 349;
a heavy chain framework sequence 2 (VH-FR2) selected from the group consisting of SEQ ID NOs:350 to 353;
a heavy chain framework sequence 3 (VH-FR3) selected from the group consisting of SEQ ID NOs:354 to 360; and
a heavy chain framework sequence 4 (VH-FR4) selected from the group consisting of SEQ ID NOs:361 to 382.

6. A chimeric antigen receptor (CAR) expression construct comprising nucleic acid sequences encoding
a chimeric antigen receptor (CAR) coding sequence comprising an ectodomain sequence comprising an antibody or antigen binding fragment thereof that binds to chondroitin sulfate proteoglycan 4 (CSPG4) comprising
variable light chain sequence comprising light chain complementarity determining regions (“LCDRs”) LCDR1 to LCDR3 sequences set forth in SEQ ID NOs: 1-3
a light chain framework sequence 1 (VL-FR1) selected from the group consisting of SEQ ID NOs:153 to 221;
a light chain framework sequence 2 (VL-FR2) selected from the group consisting of SEQ ID NOs:222 to 225;
a light chain framework sequence 3 (VL-FR3) selected from the group consisting of SEQ ID NOs:226 to 249; and
a light chain framework sequence 4 (VL-FR4) selected from the group consisting of SEQ ID NOs:250 to 255;
a variable heavy chain sequence comprising heavy chain complementarity determining regions (“HCDRs”) HCDR1 to HCDR3 sequences set forth in SEQ ID NOs: 4 to 6 and
a heavy chain framework sequence 1 (VH-FR1) selected from the group consisting of SEQ ID NOs:256 to 349;
a heavy chain framework sequence 2 (VH-FR2) selected from the group consisting of SEQ ID NOs:350 to 353;
a heavy chain framework sequence 3 (VH-FR3) selected from the group consisting of SEQ ID NOs:354 to 360; and
a heavy chain framework sequence 4 (VH-FR4) selected from the group consisting of SEQ ID NOs:361 to 382;
a transmembrane domain sequence; and
an endodomain sequence.

7. The chimeric antigen receptor (CAR) expression construct of claim 6, wherein said transmembrane domain is selected from the group consisting of CD28 (Gene ID:940, 12487), CD3-ζ (Gene ID:919; 12503 CD247), CD4 (Gene ID:920, 12504), CD8 (Gene ID:924, 12525), CD16 (Gene ID:2214; 14131; Fcgr3), NKp44 (Gene ID:9436, NCR2), NKp46 (Gene ID:9437, 17086, NCR1), and NKG2d (Gene ID:22914; 27007 KLRK1).

8. The chimeric antigen receptor (CAR) expression construct of claim 6, wherein said endodomain sequence is selected from the group consisting of CD28 (Gene ID:940), TNF receptor superfamily member 9 (Gene ID 3604, e.g., 4-1BB or CD137), CD247 (Gene ID 919, CD3-ζ), 2B4 (Gene ID:51744, CD244), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

9. The chimeric antigen receptor (CAR) expression construct of claim 6, further comprising sequences encoding a protein sequence for a transcriptional activator in the Wnt signaling pathway.

10. The chimeric antigen receptor (CAR) expression construct of claim 9, wherein said expression construct encodes a polyprotein comprising said protein sequence for a transcriptional activator in the Wnt signaling pathway and up to three additional protein coding sequences.

11. The chimeric antigen receptor (CAR) expression construct of claim 9, wherein said protein sequence for a transcriptional activator in the Wnt signaling pathway and up to three additional protein coding sequences are separated by an autonomous intra-ribosomal self-processing peptide.

12. The chimeric antigen receptor (CAR) expression construct of claim 11, wherein said autonomous intra-ribosomal self-processing is a foot-and-mouth disease virus (FMDV) 2A sequence or a related cis acting hydrolase element (CHYSEL).

13. The chimeric antigen receptor (CAR) expression construct of claim 9, wherein said transcriptional activator is selected from the group consisting of lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) and TLE family member 1, transcriptional corepressor (TLE 1, Gene ID 7088).

14. The chimeric antigen receptor (CAR) expression construct of claim 13, wherein said LEF1 selected from the group consisting of Reference Sequence (RefSeq) ID NOs: NP_057353.1, NP_001124185.1, and NP_001124186.1.

15. The chimeric antigen receptor (CAR) expression construct of claim 10, further comprising at least one protein coding sequence for a growth factor.

16. The chimeric antigen receptor (CAR) expression construct of claim 15, wherein said growth factor is selected from the group consisting of interleukin-15 (IL-15), interleukin-7 (IL-7), interleukin-12 (IL-12), interleukin-18 (IL-18), interleukin-21 (IL-21), interleukin-27 (IL-27), interleukin-33 (IL-33), and combinations thereof.

17. The chimeric antigen receptor (CAR) expression construct of claim 15, wherein said protein coding sequence for a growth factor is separated from said CAR coding sequence by a foot-and-mouth disease virus (FMDV) 2A sequence or a FMDV 2A related cis acting hydrolase element (CHYSEL) sequence.

18. The chimeric antigen receptor (CAR) expression construct of claim 6, wherein said ectodomain sequences further comprises a spacer domain.

19. The chimeric antigen receptor (CAR) expression construct of claim [00128], wherein said endodomain comprises the signal sequence of 4-1BB fused in-frame to a CD3-zeta chain.

20. The chimeric antigen receptor (CAR) expression construct of claim [00128], further comprising a DNA sequence encoding a small hairpin RNA (shRNA) sequence targeting an WIC class I or WIC class II gene, wherein the shRNA sequence is embedded in an artificial microRNA (amiR) scaffold.
